(19) United States
(12) Patent Application Publication
(10) Pub. No.: US 2026/0256818 A1
(43) Pub. Date: Sep. 3, 2026

(54) NOVEL CO-DRUG, CO-ADMINISTRATION, AND SEQUENTIAL ADMINISTRATION OF BISPECIFIC RBP4/TTR LIGAND AND C20-D3-RETINOL FOR ELIMINATION OF MECHANISM-BASED OCULAR ADVERSE EFFECTS IN TREATING MACULAR DEGENERATION AND TTR AMYLOIDOSIS

(71) Applicants: The Trustees of Columbia University in the City of New York, New York, NY (US); Rensselaer Polytechnic Institute, Troy, NY (US)

(72) Inventors: Konstantin Petrukhin, New Windsor, NY (US); Christopher L. Cioffi, Troy, NY (US)

(73) Assignees: The Trustees of Columbia University in the City of New York, New York, NY (US); Rensselaer Polytechnic Institute, Troy, NY (US)

(21) Appl. No.: 19/427,445

(22) Filed: Dec. 19, 2025

Related U.S. Application Data
(63) Continuation of application No. PCT/US2024/034823, filed on Jun. 20, 2024.
(60) Provisional application No. 63/509,184, filed on Jun. 20, 2023.

(51) Int. Cl. A61K 31/695; A61K 31/015; A61K 31/07; A61K 31/12; A61K 31/402; A61K 31/4439; A61K 31/506; A61P 27/02; C07D 207/09; C07D 401/04; C07D 403/04; C07D 403/14; C07D 409/14; C07D 487/10; C07F 7/18

(52) U.S. Cl. CPC A61K 31/695; A61K 31/015; A61K 31/07; A61K 31/12; A61K 31/402; A61K 31/4439; A61K 31/506; A61P 27/02; C07D 207/09; C07D 401/04; C07D 403/04; C07D 403/14; C07D 409/14; C07D 487/10; C07F 7/1804

(57) ABSTRACT

New therapies for macular degeneration and TTR amyloidosis are provided based on a co-drug that represents a conjugate of two distinct chemical entities as well as co-administration and sequential administration of the two chemical entities. The first component (“bispecific RBP4/TTR ligand”) is a chemical entity that engages TTR (transthyretin) and RBP4 (retinol binding protein 4) of the RBP4-TTR complex, which is involved in delivery of retinol to the retina. This component reduces traffic of retinol from circulation to the retina and provides stabilization of TTR tetramers. The second component (“C20-D3-visual-chromophore-producing compound”) is a C20-D3-modified retinoid or carotenoid that upon metabolism in a mammal can eventually produce a C20-D3 visual chromophore that represents C20-D3-9-cis-retinaldehyde or C20-D3-11-cis-retinaldehyde in the retina. Deuteration at the C20 position reduces the formation of lipofuscin bisretinoid while other functions (such as providing a precursor for in vivo synthesis of the visual chromophore, 11-cis-retinaldehyde) are not reduced.

This application is a continuation of PCT International Application Number PCT/US2024/034823, filed Jun. 20, 2024, claiming benefit of, U.S. Provisional Application No. 63/509,184, filed Jun. 20, 2023, the contents of each of which are hereby incorporated by reference into the subject application.

This invention was made with government support under grant number EY028549 awarded by the National Institutes of Health. The government has certain rights in the invention.

Throughout the present disclosure, certain publications are referenced in parentheses. Full citations for these publications may be found immediately preceding the claims. The disclosures of these publications in their entireties are hereby incorporated by reference into the present disclosure in order to describe more fully the state of the art to which the present disclosure relates.

### BACKGROUND OF THE INVENTION

Macular degenerations affect millions in the United States alone, with the loss of central vision greatly affecting living conditions. Many forms of the disease exist, with the most common being age-related macular degeneration (AMD). Although some treatments exist, particularly for the subset known as wet AMD, treatment is still limited for dry AMD to supportive care to delay the onset of the disease where the supportive care relates to the use of AREDS (Age-Related Eye Disease Study) formula of vitamins and nutrients. While the FDA (Food and Drug Administration) recently approved intravitreal pegcetacoplan (SYFOVRE™; Apellis Pharmaceuticals, Inc.) for treating geographic atrophy (GA) secondary to dry AMD, the treatment is based on a pegylated peptide that is a complement inhibitor. Specifically, pegcetacoplan binds to complement protein C3 and its activation fragment C3b with high affinity, thereby regulating the cleavage of C3 and the generation of downstream effectors of complement activation. The treatment is also associated with inconvenient intraocular delivery, and its efficacy is not optimal. Oral therapies, such as co-drug therapies, will have a significant advantage over pegcetacoplan in terms of patient convenience. They further have a mechanism that is independent of direct modulation of the complement cascade in the retina. An attractive target for treatment of the disease is the downregulation of serum retinol, but current approaches cause adverse events in patients, limiting their usage.

Photoreceptor loss in dry AMD is secondary to abnormalities in the Retinal Pigment Epithelium (RPE), a cellular layer that provides critical metabolic support to rods and cones. Age-dependent accumulation of lipofuscin in the RPE is associated with age-related increased incidences of dry AMD and may be one of several pathogenic factors contributing to disease onset and progression. Bisretinoids, which are byproducts of the visual cycle stemming from retinaldehyde dimerization, mediate lipofuscin toxicity and exert a variety of deleterious effects on RPE cells (Bergmann, M. et al. 2004; Sparrow, J. R. et al. 2003; Dorey, C. K. et al. 1989; Sparrow, J. R. & Boulton, M. 2005). Among them is the dysregulation of the complement system (Charbel, I. P. et al. 2015; Radu, R. A. et al. 2011; Radu, R. A. et al. 2014; Brandstetter, C. et al. 2015A) and inflammasome activation (Brandstetter, C. et al. 2015A; Brandstetter, C. et al. 2015B), the hallmarks of dry AMD pathology (Geerlings, M. J. et al. 2017).

While the accumulation of lipofuscin is one of several contributing factors underlying dry AMD pathogenesis, enhanced bisretinoid biosynthesis represents the sole causative factor underlying Stargardt disease (STGD1), a genetic form of macular degeneration (Birnbach, C. D. et al. 1994; De Laey, J. J. & Verougstraete, C. 1995; Delori, F. C. 1995; Eagle, R. C. et al. 1980). The non-enzymatic biosynthesis of cytotoxic bisretinoids involves two condensations of all-trans-retinaldehyde (Sparrow, J. R. et al. 2003) and/or 11-cis-retinaldehyde with phosphatidylethanolamine (Boyer, N. P. et al. 2012; Tang, P. H. et al. 2012). Retinaldehyde synthesis and bisretinoid production are fueled by the influx of serum retinol to the retina. Thus, the pharmacological down-regulation of serum retinol may limit retinal bisretinoid and retinaldehyde production, and represents a highly attractive target area for dry AMD and STGD1 treatment (Kennedy, C. J. et al. 1995; Radu, R. A. et al. 2005; Radu, R. A. et al. 2003; Maeda, A. et al. 2006; Palczewski, K. 2010; Cioffi, C. L. et al. 2014; Dobri, N. et al. 2013). Serum retinol is delivered to the retina from circulation in a tertiary complex with Retinol-Binding Protein 4 (RBP4) and transthyretin (TTR). Without interacting with TTR, holo-RBP4 is rapidly cleared via glomerular filtration. Given that the RBP4-TTR interaction is retinol-dependent, compounds antagonizing retinol binding to RBP4 may also induce dissociation of the RBP4-TTR complex with a subsequent reduction in circulating levels of retinol and RBP4.

Bispecific RBP4/TTR ligands are highly effective in lowering serum RBP4 (Cioffi, C. L. et al. 2020) and in reducing bisretinoid synthesis in relevant mouse models (International Patent Application No. PCT/US2021/042300, published as WO 2022/020305 A2). However, human clinical use of bispecific RBP4/TTR ligands may be associated with mechanism-based ocular adverse effects (AEs), even though no ocular adverse effects were induced by RBP4 antagonists in mice, as previously reported (Dobri, N. et al. 2013; Racz, B. et al. 2018). Human clinical data indicates that long-term use of fenretinide (a prototypical RBP4 antagonist) in cancer patients was associated with transient and reversible ocular AEs, such as diminished dark adaptation, in a subset of patients (yearly prevalence: 5.8-6.7%) (Camerini, T. et al. 2001). Similarly, a Phase 2 fenretinide trial in patients with dry AMD reported reversible reduction in dark adaptation in ~10% of drug-treated patients (no ocular AEs with placebo) (Mata, N. L. et al. 2013). Tinlarebant (another RBP4 antagonist) safety data presented by Belite Bio indicates that a subset of patients may develop asymptomatic delayed dark adaptation (measured only instrumentally) or symptomatic xanthopsia (abnormalities in cone vision).

### SUMMARY OF THE INVENTION

The present disclosure provides two-component co-drug, co-administration, and sequential-administration strategies that combine the ability to reduce retinol while simultaneously mitigating the adverse events associated with loss of retinol. The co-drug, as a representative example, is formed from the conjugation of a RBP4-lowering compound with a deuterated retinol. The first component (“bispecific RBP4/TTR ligand”) treats the disease by limiting delivery of retinol while the second component (“C20-D3-visual-chromophore-producing compound”) suppresses production of additional lipofuscin bisretinoid and reduces the adverse effects associated with partial reduction in production of the visual chromophore, 11-cis-retinaldehyde, in the retina. As such, the present disclosure provides means to overcome the toxicity associated with current macular degeneration treatments while still maintaining the pharmacological effects.

The first component, a “bispecific RBP4/TTR ligand,” is a chemical entity that engages the RBP4 and the TTR of the RBP4-TTR complex, which is involved in delivery of retinol to the retina. The functional purpose of this component is to partially reduce traffic of retinol from circulation to the retina and to provide stabilization of TTR tetramers.

Specifically, a “bispecific RBP4/TTR ligand” is a compound having the structure:

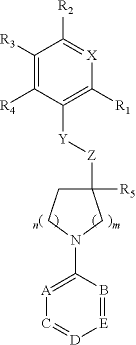

wherein
   X is CR₆ or N;
   R₁, R₂, R₃, R₄, and R₆ are each independently —H, —F, —Cl, —Br, —I, —NO₂, —CN, —CF₃, —CF₂H, —OCF₃, -(alkyl), -(haloalkyl), -(alkenyl), -(alkynyl), -(aryl), -(heteroaryl), -(cycloalkyl), -(cycloalkylalkyl), -(heteroalkyl), heterocycle, heterocycloalkyl, -(alkylheteroalkyl), -(alkylaryl), —OH, —OAc, —O-(alkyl), —O-(alkenyl), —O-(alkynyl), —O-(aryl), —O-(heteroaryl), —SH, —S-(alkyl), —S-(alkenyl), —S-(alkynyl), —S-(aryl), —S-(heteroaryl), —NH₂, —NH-(alkyl), —NH-(alkenyl), —NH-(alkynyl), —NH-(aryl), —NH-(heteroaryl), —C(O)R₇, —S(O)R₇, —SO₂R₇, —NHSO₂R₇, —OC(O)R₇, —SC(O)R₇, —NHC(O)R₈, or —NHC(S)R₈, wherein R₇ is H, -(alkyl), —OH, —O(alkyl), —NH₂, —NH(alkyl), or —N(alkyl)₂, and
wherein R₈ is -(alkyl), —O-(alkyl), —NH₂, —NH(alkyl), or —N(alkyl)₂;
   Y is O, S, N, NH, or a bond;
   Z is O, S, N, NH, (CH₂)ₒ, or a bond;
   R₅ is H, OH, halogen, or alkyl, or R₅ is (CH₂)ₚ and is bound to Y when Y is N to form a ring together with Z;
   o and p are independently 0, 1, 2, or 3;
   m and n are independently 0, 1, 2, 3, or 4;
   A, C, and D are each independently N or CR₉;
   R₉ is H, halogen, —OH, alkyl, cycloalkyl, cycloalkylalkyl, —O-(alkyl), —S-(alkyl), —NH₂, —NH(alkyl), —NH(alkyl)₂, —CO₂H, or —CO(O-alkyl);
   B and E are N, CR₉, or C—F-G wherein at least one of B or E is C—F-G;
   F is absent or present, and when present, is

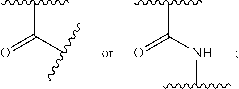

and
G is H, substituted or unsubstituted monocycle, bicycle, heteromonocycle, heterobicycle, aryl, heteroaryl, alkyl, cycloalkyl, cycloalkylalkyl, CO₂H, COOR₁₀, OH, OR₁₀, NH₂, NHR₁₀, NR₁₀R₁₁, SO₂ (alkyl), SO₂(cycloalkyl), SO₂(cycloalkylalkyl), CH₂NHR₁₀, CH₂NR₁₀R₁₁, or CH₂COOR₁₀, wherein R₁₀ and R₁₁ are each independently H, alkyl, cycloalkyl, —C(O)-alkyl, —C(O)-cycloalkyl, —C(O)OH, —C(O)—O-alkyl, —C(O)—O-cycloalkyl, —C(O)NH₂, —C(O)NH(alkyl), —C(O)NH(cycloalkyl), —C(O)N(alkyl)₂, —CH₂NH (alkyl), —CH₂COOH, —SO₂CH₃, —OH, —O(alkyl), —NH₂, —NH(alkyl), or —N(alkyl)₂;
   or a pharmaceutically acceptable salt thereof.

The second component, a “C20-D3-visual-chromophore-producing compound,” is defined as any C20-D3-modified retinoid or carotenoid that upon metabolism in a mammal can eventually produce a C20-D3 visual chromophore that represents C20-D3-9-cis-retinaldehyde or C20-D3-11-cis-retinaldehyde in the retina. Examples of a C20-D3-visual-chromophore-producing compound include C20-D3-retinol, which is a deuterated form of vitamin A (retinol), C20-D3-retinaldehyde, C20-D3-retinyl esters (such as C20-D3-retinyl acetate), C20-D3-9-cis-retinol, C20-D3-9-cis-retinaldehye, C20-D3-9-cis-retinyl esters (such as C20-D3-9-cis-retinyl acetate), C20-D3-11-cis-retinol, C20-D3-11-cis-retinaldehye, C20-D3-11-cis-retinyl esters (such as C20-D3-11-cis-retinyl acetate), and C20-D3-C20′-D3-β-carotene. The deuteration at the C20 position reduces the formation of lipofuscin bisretinoid while other functions (such as providing a precursor for in vivo synthesis of the visual chromophore, 11-cis-retinaldehyde) are not reduced. Additional deuterations at positions other than C20 are possible.

When absorbed in the body of a mammal, the C20-D3-visual-chromophore-producing compound initially generates C20-D3-retinol in a form of one of three stereoisomers: C20-D3-all-trans-retinol, C20-D3-9-cis-retinol, and C20-D3-11-cis retinol. Upon delivery to the retina, deuterated all-trans-retinol undergoes isomerization by retinoid isomerohydrolase RPE65 to form 11-cis-retinol which in turn is converted to 11-cis-retinaldehyde, a visual chromophore. In contrast, cis retinoids can directly produce the visual chromophore without requiring this isomerization reaction. The visual chromophore can be either 11-cis-retinaldehyde (a natural chromophore) or 9-cis-retinaldehyde (an artificial chromophore that can bind to opsin and form rhodopsin similarly to 11-cis-retinaldehyde).

Various ratios of the first component to the second component may be used. As a co-drug, the ratio may be a 1:1 conjugate in its simplest form. The co-drug may be designed following general conjugating approaches developed for the antibody-drug conjugate (ADC) system. However, in contrast to ADC, the co-drug of the present disclosure is designed to degrade and release the first and second components in the gastrointestinal (GI) tract, or via hydrolysis in blood or lymphatic circulation, not in the target cell as is typical for ADC. Moreover, the co-drug of the present disclosure is designed to be used orally, while ADC therapies need to be administered by non-oral routes (such as intravenously or subcutaneously).

The first component (bispecific RBP4/TTR ligand) will limit the RBP4-mediated delivery of vitamin A to the retina to inhibit bisretinoid production. In addition, the first component will stabilize circulating TTR tetramers, preventing their dissociation and thus inhibiting the amyloidogenic cascade.

Regarding the second component (C20-D3-visual-chromophore-producing compound), the fate of the released C20-D3-retinol will be as follows. It is known that 25-33% of postprandial retinoid-laden chylomicrons are taken by extrahepatic vitamin A-dependent tissues such as the retina (Vogel, S. et al. 2002). Like dietary retinoids, C20-D3-retinol will be esterified in the GI tract, packaged in chylomicrons, and delivered to the retina. Consistent chylomicron delivery of C20-D3-retinoids to the retina will provide a reliable supply of retinoids for the synthesis of visual chromophore (11-cis-retinal) and rhodopsin in visual cycle reactions (Charbel, I. P. et al. 2015), thus partially compensating for the blocking of the RBP4-mediated route and reducing related ocular AEs. At the same time, C20-D3-retinoids will not contribute to the biosynthesis of bisretinoids due to a kinetic isotope effect that slows bisretinoid synthesis (Charbel, I. P. et al. 2015; Kaufman, Y. et al. 2011; Ma, L. et al. 2011). Rhodopsin represents a protein called opsin conjugated with the visual chromophore called 11-cis-retinaldehyde; 9-cis-retinaldehyde can also serve as a visual chromophore in place of 11-cis-retinaldehyde. Apart from rhodopsin, there are three cone opsins (blue, red, and green opsins) expressed in three types of cone photoreceptors (blue, red, and green cones). The co-drug, co-administration, and sequential administration of the present disclosure provide visual chromophores to supplement all four types of functional opsins (rhodopsin, blue-opsin, green-opsin, and red-opsin).

The first and second components may also be co-administered as separate chemical entities simultaneously, contemporaneously, or concomitantly (that is, without being conjugated or bonded to each other), or they may be administered sequentially in a suitable order in which the first component is administered first or the second component is administered first. In addition, the second component (C20-D3-visual-chromophore-producing compound) may be administered in a form packaged in chylomicrons.

In the co-drug and co-administration approaches of the present disclosure, significantly improved patient compliance can be expected. The co-drug approach will also provide more predictable pharmacokinetics (PK) and pharmacodynamics (PD), and may further avoid potential adverse drug-drug interactions (DDIs) that could arise from multiple drug intakes.

### BRIEF DESCRIPTION OF THE FIGURES

FIG. 1. All-trans-retinol (vitamin A) (1), retinoic acid (2) (a vital retinoid involved in morphogenesis), 11-cis-retinal (3) (a key retinoid required for phototransduction), and the thyroid hormone thyroxine (T4) (4).

FIG. 2. Examples of previously reported RBP4 antagonists that include fenretinide (5), A1120 (6), non-retinoid [3.3.0]-octahydrocyclopenta[c]pyrrolo RBP4 antagonist 7, BPN-14136 (8), [3.3.0]-octahydrocyclopenta[c]pyrrolo RBP4 antagonist 9, and [1,2,4]triazolo[4, 3-a]pyridine RBP4 antagonist 10.

FIG. 3. Examples of reported small molecule TTR tetramer kinetic stabilizers that include tafamidis (11), AG10 (12), diflunisal (13), and tolcapone (14).

FIG. 4. Bispecific RBP4/TTR ligands for treating dry AMD-SSA comorbidity. (A) Schematics illustrating use of a selective RBP4 antagonist for disrupting retinol-dependent RBP4-TTR interaction and inducing serum RBP4-retinol reduction. The release of unliganded TTR induces its aggregation and leads to SSA. (B) A bispecific antagonist induces serum RBP4-retinol reduction and also acts as a kinetic stabilizer of released TTR tetramers, preventing TTR amyloid fibril formation.

FIG. 5. Syntheses of C20-D3-retinol (9) and C20-D3-retinyl acetate (10) (Bergen, H. R. et al. 1988).

FIG. 6. Synthesis of C20-D3-9-cis-retinol based on existing routes for synthesizing 9-cis-retinol (Korean Patent No. 10-2271364 B1) and for incorporating deuterium at the C20 position (Bergen, H. R. et al. 1988).

FIG. 7. Synthesis of C20-D3-11-cis-retinol based on existing routes for synthesizing 11-cis-retinol (Borhan, B. et al. 1999) and for incorporating deuterium at the C20 position (O'Broin, C. Q. & Guiry, P. J. 2020).

FIG. 8. Synthesis of C20-D3-C20′-D3-β-carotene based on existing routes for synthesizing C20-D3-all-trans-retinol (Bergen, H. R. et al. 1988) and β-carotene (Goswami, B. C. & Barua, A. B. 2003).

FIG. 9. TTR amyloidogenesis cascade (from Connelly, S. et al. 2010) For amyloidogenesis to occur, an unliganded TTR tetramer must first dissociate into four folded monomers and undergo partial denaturation. These pieces then subsequently misassemble into a variety of aggregate structures including toxic amyloid fibrils. Complexation with retinol-RBP4 or binding of natural or synthetic TTR ligands stabilizes TTR tetramers and prevents amyloidogenesis.

FIG. 10. Medicinal chemistry co-drug strategy. (A) Design principles of a bispecific RBP4/TTR agent with C20-D3-retinol in a single chemical entity. (B) Examples of a co-drug core that can provide different molar ratios of bispecific RBP4/TTR ligand to C20-D3-retinol. ACPHS-52 is shown as the bispecific RBP4/TTR ligand. (C) Examples of co-drugs whereby C20-D3-retinol is linked to the cores via various low-pH and/or proteolytic cleavable linkers that are successfully used in ADC development. The bispecific RBP4/TTR ligands are linked via labile esters.

FIG. 11. A synthetic route for a 2:1 molar ratio ACPHS-52:C20-D3-retinol co-drug (Lee, B. P. et al. 2006; Keller, K. A. et al. 2005; Finniss, M. C. et al. 2014; Bergen, H. R. et al. 1988).

FIG. 12. Structure of bisretinoids A2E and isoA2E, cytotoxic components of retinal lipofuscin.

FIG. 13. Structure of bisretinoids atRAL di-PE (all-trans-retinal dimer-phosphatidyl ethanolamine) and A2-DHP-PE, cytotoxic components of retinal lipofuscin. R₁ and R₂ refer to various fatty acid constituents.

FIG. 14. Effects of C20-D3-retinyl acetate on rhodopsin levels in ACPHS-52-treated Balb/c mice. Rhodopsin was measured spectrophotometrically in retinal extracts (n=8) from dark-adapted treated and control mice. Treated mice were given 7 daily oral dose of 25 mg/kg ACPHS-52 or the same dose of ACPHS-52 along with 3.3 mg/kg C20-D3-retinyl acetate. Untreated control mice were kept on standard diet. An unpaired t-test on the ungrouped data revealed a statistically significant 1.8-fold increase in rhodopsin concentration in mice co-treated with C20-D3-retinyl acetate and ACPHS-52 in comparison to mice treated only with ACPHS-52 (P<0.05).

### DETAILED DESCRIPTION OF THE INVENTION

The present disclosure provides new therapies based on a co-drug that represents a conjugate of two distinct chemical entities, co-administration of the two chemical entities, and sequential administration of the two chemical entities.

### 1. Bispecific RBP4/TTR Ligands

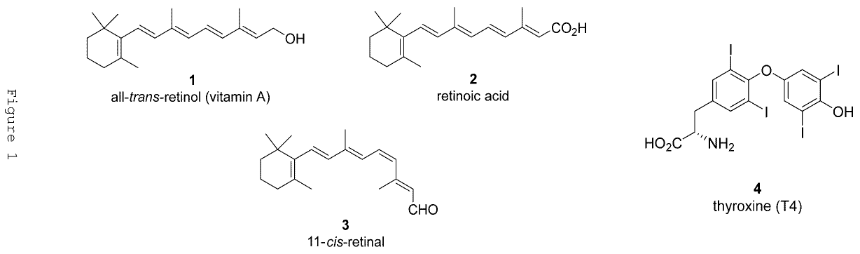

Age-related macular degeneration (AMD) is the most common cause of blindness in developed countries. Age-dependent accumulation of cytotoxic lipofuscin bisretinoids in the retina may significantly contribute to pathogenesis of the atrophic form of AMD. The essential vitamin all-trans-retinol (vitamin A, 1) (FIG. 1) serves as a precursor for the biosynthesis of retinoic acid (2) (Steinmetz, A. C. et al. 2001; Clagett-Dame, M. & DeLuca, H. F. 2002; Wolf, G. 1984), 11-cis-retinal (3) (Kiser, P. D. et al. 2014; Tsin, A. et al. 2018), and many other key retinoids involved in multiple cellular processes and numerous critical biological functions throughout the body.

Bisretinoid synthesis in the eye depends on the influx of all-trans-retinol (1) from the serum to the retina. Formation of the tertiary retinol-binding protein 4 (RBP4)-transthyretin (TTR)-retinol complex in the serum is required for this influx. Reducing circulating levels of RBP4 and 1 via selective antagonists could modulate the visual cycle, reduce the rate of cytotoxic bisretinoid formation in the retinal pigment epithelium (RPE), and halt geographic atrophy in patients with atrophic (dry) age-related macular degeneration (AMD) and Stargardt disease (Radu, R. A. et al. 2005; Palczewski, K. 2010; Petrukhin, K. 2013; Petrukhin, K. 2007).

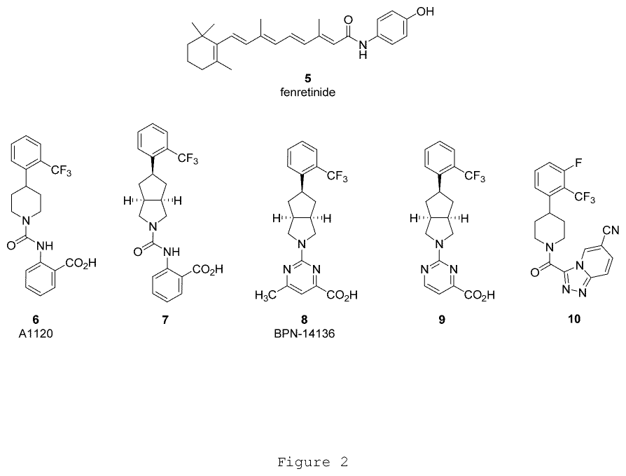

Competitive RBP4 antagonists that displace 1 prevent the formation of the holo-RBP4-TTR complex, thereby inducing reductions in circulating RBP4 and 1 levels via rapid renal clearance. A diminished influx of 1 to the RPE results in a reduction of cytotoxic bisretinoid accumulation in the retina, which is believed to underlie parts of the pathophysiology of dry AMD and Stargardt disease (Radu, R. A. et al. 2005; Dobri, N. et al. 2013; Racz, B. et al. 2018; Young, R. W. 1987; Dorey, C. K. et al. 1989; Holz, F. G. et al. 2001; Holz, F. G. et al. 1999; Holz, F. G. et al. 2007; Schmitz-Valckenberg, S. et al. 2009; Finnemann, S. C. et al. 2002; Suter, M. et al. 2000; Sparrow, J. R. et al. 2003; Sparrow, J. R. et al. 2012; Delori, F. C. 1995; Weng, J. et al. 1999; Sparrow, J. R. & Cai, B. 2001; Bergmann, M. et al. 2004; Sparrow, J. R. et al. 1999; De, S. & Sakmar, T. P. 2002; Vives-Bauza, C. et al. 2008; Zhou, J. et al. 2006; Radu, R. A. et al. 2011; Ben-Shabat, S. et al. 2002; Rozanowska, M. et al. 1995; Sparrow, J. R. et al. 2002; Dontsov, A. E. et al. 2009). The approach is supported by proof-of-concept data obtained for fenretinide (5) (FIG. 2), which was studied in the preclinical Abca4^{-/-} transgenic mouse model of enhanced retinal lipofuscinogenesis (Radu, R. A. et al. 2005) and in an extended Phase II clinical trial with dry AMD patients (Berni, R. & Formelli, F. 1992; Adams, W. R. et al. 1995; Mata, N. L. et al. 2013). The non-retinoid RBP4 antagonist A1120 (6) (Motani, A. et al. 2009) was found to lower circulating RBP4 plasma levels in rodents by >70% and reduce retinal bisretinoid accumulation in Abca4^{-/-} mice (Dobri, N. et al. 2013). Selective and orally bioavailable non-retinoid RBP4 antagonists 7 (Cioffi, C. L. et al. 2014) and BPN-14136 (8) (Cioffi, C. L. et al. 2015) displayed favorable pharmacokinetic (PK) profiles and induced dose-dependent reductions in rodent plasma RBP4 levels in both acute and chronic dosing studies. Compound 8 also robustly lowered serum RBP4 levels and exhibited excellent pharmacokinetic-pharmacodynamic (PK/PD) correlations in non-human primates upon oral administration (Racz, B. et al. 2020). Compound 8 inhibited lipofuscin bisretinoid synthesis with concomitant normalization of retinal complement system protein expression in the Abca4^{-/-} mouse model without altering visual cycle kinetics at doses inducing maximal serum RBP4 reduction (Racz, B. et al. 2018).

In addition to transporting 1 to targeted tissues, RBP4 has also been identified as an adipokine, and epidemiological evidence suggests that moderately elevated levels of the protein positively correlate with type 2 diabetes (Graham, T. E. et al. 2006; Yang, Q. et al. 2005), obesity (Aeberli, I. et al. 2007), insulin resistance (Kowalska, I. et al. 2008), cardiovascular disease (Ingelsson, E. et al. 2009; Qi, Q. et al. 2007; Norseen, J. et al. 2012), and hepatic steatosis (Lee, S. A. et al. 2016). Thus, the pharmacological reduction of circulating RBP4 serum levels may also hold promise for the treatment of a myriad of metabolic diseases. It has recently been reported that RBP4 antagonist 10 significantly lowered serum RBP4 levels in rodents (>80%), reduced the concentration of circulating RBP4 produced in the adipose tissue, and demonstrated efficacy in the transgenic adi-hRBP4 murine model of hepatic steatosis, suggesting that it may have therapeutic utility for the treatment of non-alcoholic fatty liver disease (NAFLD) (Cioffi, C. L. et al. 2019).

The deposition of amyloid aggregates derived from either mutant (TTRm) or wild-type (TTRwt) underlies TTR amyloidosis (ATTR) diseases such as senile systemic amyloidosis (SSA), peripheral polyneuropathy (ATTR-PN), and cardiomyopathy (ATTR-CM) (Johnson, S. M. et al. 2005; Foss, T. R. et al. 2005; Falk, R. H. et al. 1997; Brunjes, D. L. et al. 2016; Ton, V. K. et al. 2014). The breakage of the dimer-dimer interface in TTR tetramers constitutes the first step in the TTR tetramer dissociation process that leads to TTR misfolding. Approximately 50% of serum TTR is associated with holo-RBP4, and the formation of the tertiary holo-RBP4-TTR complex is suggested to stabilize this fraction of serum TTR tetramers, protecting them from dissociation and misfolding (White, J. T. & Kelly, J. W. 2001; Hyung, S. J. et al. 2010). Based on the in vitro observation that RBP4-TTR interaction is capable of conferring an additional stabilization to tetrameric TTR (White, J. T. & Kelly, J. W. 2001; Hyung, S. J. et al. 2010), it seems plausible that the release of TTR tetramers from RBP4-TTR-retinol complexes induced by selective RBP4 antagonists may lead to tetramer destabilization and its enhanced dissociation to dimer subunits. The resulting dimers may then further dissociate into monomers that can misfold, aggregate, and/or oligomerize, and eventually form insoluble TTR amyloid fibrils (White, J. T. & Kelly, J. W. 2001; Hyung, S. J. et al. 2010).

While selective RBP4 antagonists can be a safe and effective therapy for the majority of dry AMD patients, this class of compounds may potentially be counter-indicated for a fraction of AMD patients who may be prone to developing ATTR. In addition to individuals with rare genetic forms of transthyretin amyloidosis caused by proamyloidogenic TTR mutations, the use of selective RBP4 antagonists may not be optimal in patients with senile systemic amyloidosis (SSA), a late-onset non-genetic disease associated with misfolding and aggregation of wild-type TTR. SSA affects approximately 25% of patients over the age of 80 (Ruberg, F. L. & Berk, J. L. 2012; Connors, L. H. et al. 2011; Westermark, P. et al. 2003), and based on the high population frequency of this disease and dry AMD, significant comorbidity between the two conditions is expected. In addition, the use of selective RBP4 antagonists may not be optimal in older African-American patients with dry AMD who have the increased chance of carrying a relatively high-frequency pro-amyloidogenic V122I mutation in the TTR gene (Buxbaum, J. N. et al. 2017; Alexander, K. M. & Falk, R. H. 2016). It is undesirable for an effective chronic treatment for one of the two conditions to be counter-indicated for the use in patients with another one, and developing an optimal therapy for dry AMD that can be safely used in patients with ATTR comorbidities is an important objective.

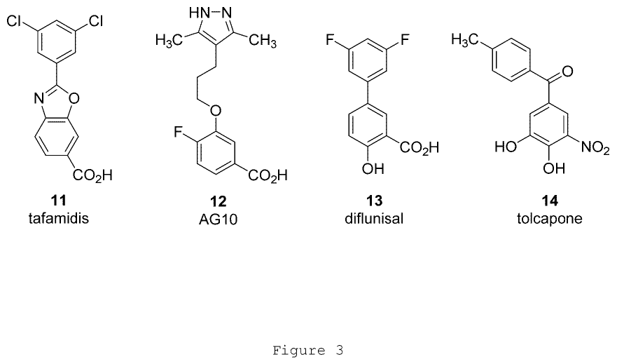

The initial and rate-limiting step in ATTR pathophysiology is the sequential dissociation of TTR tetramers (Johnson, S. M. et al. 2005; Foss, T. R. et al. 2005). While thyroxine (4) (FIG. 1) binding was reported to stabilize TTR tetramers (Sekijima, Y. et al. 2003), the majority of TTR in circulation (up to 90%), including TTR in a complex with holo-RBP4, is not bound to its natural ligand (White, J. T. & Kelly, J. W. 2001). Current therapeutic approaches to treat ATTR-CM include small molecule kinetic stabilizers of TTR tetramers that bind at the T4 binding sites and increase the energy barrier of tetramer dissociation (Kerschen, P. & Plante-Bordeneuve, V. 2016; Almeida, M. R. et al. 2005; Almeida, M. R. et al. 2004; Johnson, S. M. et al. 2005; Nencetti, S. & Orlandini, E. 2012; Adams, D. et al. 2016). Two orally bioavailable kinetic stabilizers clinically investigated to date include FDA-approved tafamidis (11) (FIG. 3) (Coelho, T. et al. 2016; Nencetti, S. et al. 2013; Lamb, Y. N. & Deeks, E. D. 2019; Bulawa, C. E. et al. 2012), and AG10 (12) (Penchala, S. C. et al. 2013; Miller, M. et al. 2018). TTR stabilizer 11 is currently approved to treat familial amyloid polyneuropathy (FAP) and ATTR-CM patients, and 12 has demonstrated near-complete stabilization of TTR in ATTR-CM patients with symptomatic, chronic heart failure in Phase III clinical trials (Judge, D. P. et al. 2019). In addition, the repurposed FDA-approved non-steroidal anti-inflammatory drug (NSAID) diflunisal (13) (Berk, J. L. et al. 2012) and catechol-O-methyl transferase (COMT) inhibitor tolcapone (14) (Sant'Anna, R. et al. 2016) are examples of additional small molecules that also exhibit TTR tetramer kinetic stabilization activity and have been investigated for clinical efficacy against ATTR-PN.

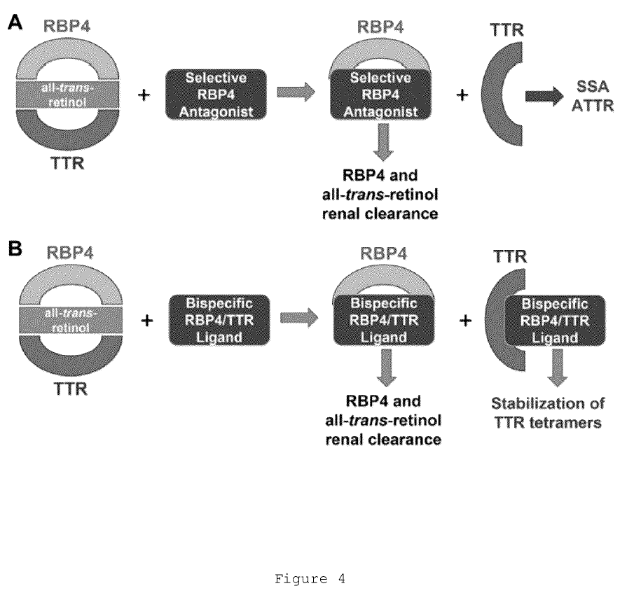

International Patent Application No. PCT/US2021/042300, published as WO 2022/020305 A2 and hereby incorporated by reference in its entirety, discloses a class of non-retinoid bispecific compounds capable of exhibiting dual retinol-binding protein 4 (RBP4) antagonist and transthyretin (TTR) tetramer kinetic stabilization activity for the treatment of dry age-related macular degeneration (AMD) and TTR amyloidosis (ATTR) comorbidities. These compounds, defined as “bispecific RBP4/TTR ligands” in the present disclosure, have applications for providing therapeutic benefits associated with reducing circulating RBP4 levels while simultaneously stabilizing unliganded TTR tetramers released from the holo-RBP4-TTR complex, thus circumventing potential risks of amyloid fibril formation as schematized in FIG. 4. Examples of bispecific RBP4/TTR ligands include

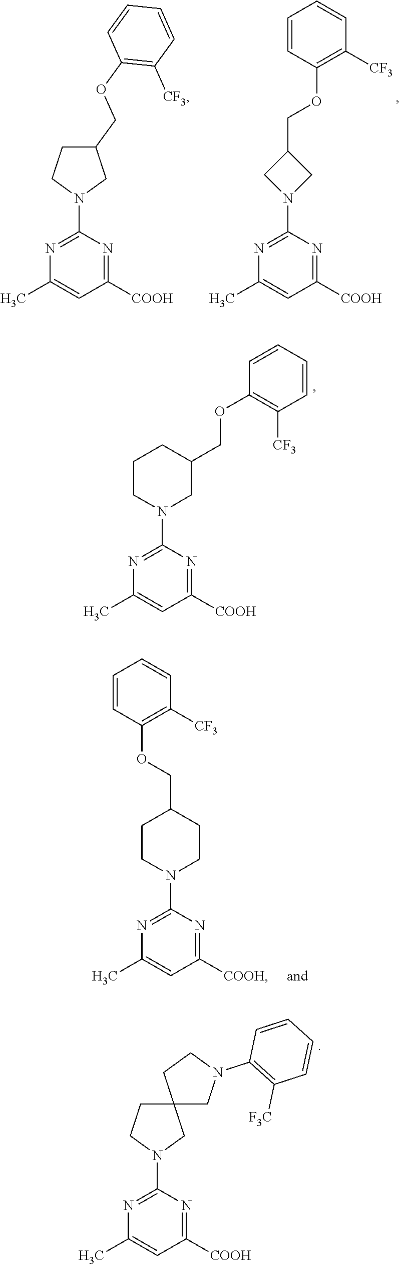 [SMILES: Cc1cc(C(=O)O)nc(N2CCC(COc3ccccc3C(F)(F)F)C2)n1 ; Cc1cc(C(=O)O)nc(N2CC(COc3ccccc3C(F)(F)F)C2)n1 ; Cc1cc(C(=O)O)nc(N2CCCC(COc3ccccc3C(F)(F)F)C2)n1 ; Cc1cc(C(=O)O)nc(N2CCC(COc3ccccc3C(F)(F)F)CC2)n1 ; Cc1cc(C(=O)O)nc(N2CCC3(CCN(c4ccccc4C(F)(F)F)C3)C2)n1]

Namely, bispecific RBP4/TTR ligands combine antagonism of retinol-dependent RBP4-TTR interaction (which translates into partial serum retinol lowering, reduction of bisretinoid and retinaldehyde toxicities, and normalization of complement system dysregulation in the retina) and kinetic stabilization of TTR tetramers (which inhibits the TTR amyloidogenic cascade in vivo). Furthermore, a polypharmacological approach relying on single bispecific molecules capable of exhibiting dual activity for both targets presents advantages over combined administration of separate single agents for each target. Such advantages include improving patient compliance, providing more predictable PK and PD, and avoiding potential adverse drug-drug interactions (DDIs) that could arise from multiple drug intakes (Rodrigues, D. A. 2008). In particular, a distinct and significant advantage over tafamidis is that unlike tafamidis, bispecific RBP4/TTR ligands do not confer inhibitory activity at BCRP (breast cancer resistance protein) and OAT (organic anion transporter)-1 transporters, which are involved in clearance of heavily prescribed statins, further reducing the risk of undesirable DDIs for the dry AMD patient population.

### 2. C20-D3-Retinol

A critical step in bisretinoid synthesis is spontaneous dimerization of retinaldehydes conjugated to phosphatidylethanolamine (PE) via Schiff base formation in photoreceptor membranes (Washington, I. et al. 2016). A rate-determining step in retinaldehyde dimerization is the cleavage of carbon-hydrogen bonds at C20 of the retinaldehyde-PE Schiff base via a [1,6]-hydride shift (Kaufman, Y. et al. 2011).

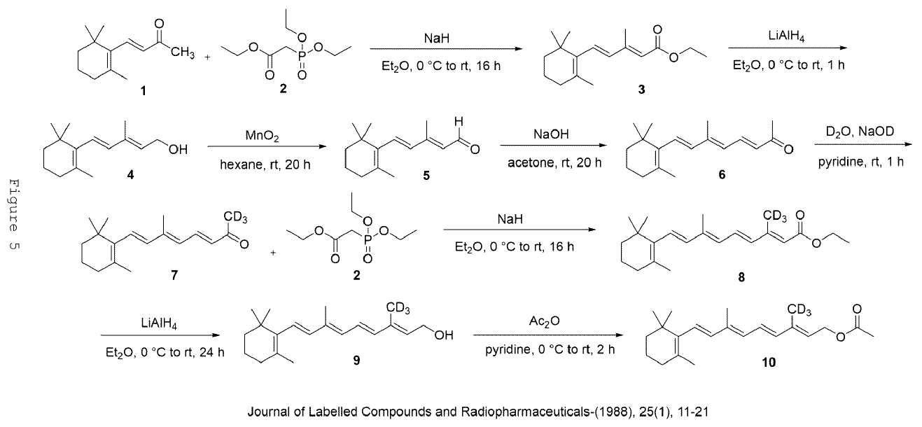

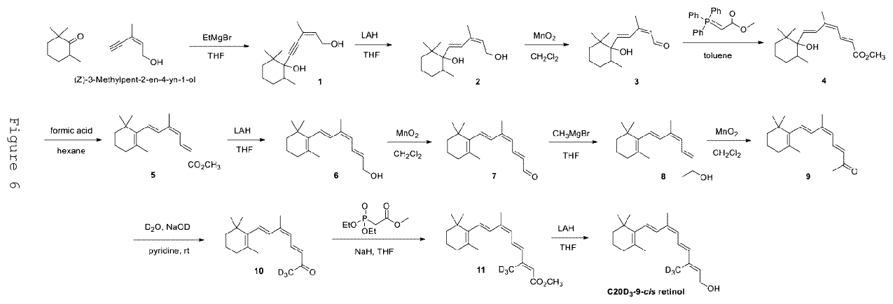

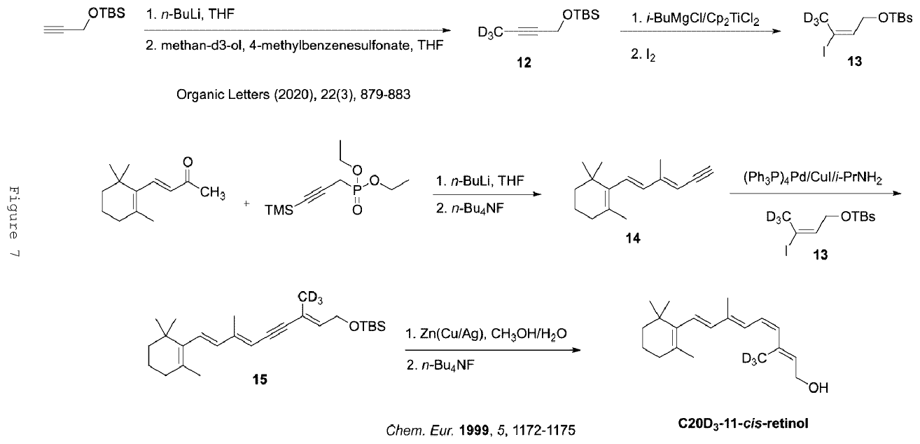

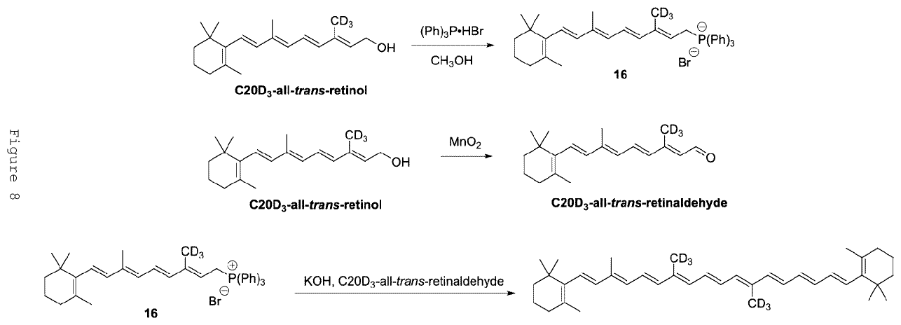

Deuterated retinoids have been used in humans for decades as traces in studies of vitamin A metabolism (Haskell, M. J. et al. 1999; Haskell, M. J. et al. 1997). FIGS. 5 through 8 show, respectively, how C20-D3-retinol, C20-D3-retinyl acetate, C20-D3-9-cis-retinol, C20-D3-11-cis-retinol, and C20-D3-C20′-D3-β-carotene may be synthesized. Introduction of deuterium at C20 of vitamin A results in a kinetic isotope effect that slows the formation of retinaldehyde dimers in vivo and in vitro (Kaufman, Y. et al. 2011; Ma, L. et al. 2011). In in vitro experiments, C20-D3-retinaldehyde formed dimers 12-times less rapidly than unlabeled retinaldehyde, while bisretinoid A2E formation was reduced by 7-fold (Kaufman, Y. et al. 2011). C20-D3-retinyl acetate was effective in inhibiting bisretinoid synthesis and normalization of complement system dysregulation in the retina of Abca4^{-/-} mice, a model of Stargardt disease (STGD1) (Charbel, I. P. et al. 2015).

Evaluation of C20-D3-retinyl acetate (under the name ALK-001) in clinical trials for dry AMD and STGD1 is ongoing (Petrukhin, K. 2020). Standing alone, the bisretinoid lowering efficacy of ALK-001 may depend on dietary restrictions in consuming standard vitamin A-containing food (Petrukhin, K. 2013). Moreover, an emerging view of dry AMD and STGD1 pathogenesis underscores the independent role of retinaldehyde toxicity in the disease pathology (Maeda, A. et al. 2012; Gliem, M. et al. 2016; Smith, R. T. et al. 2013; Sparrow, J. R. et al. 2013).

### 3. TTR Amyloidosis (ATTR)

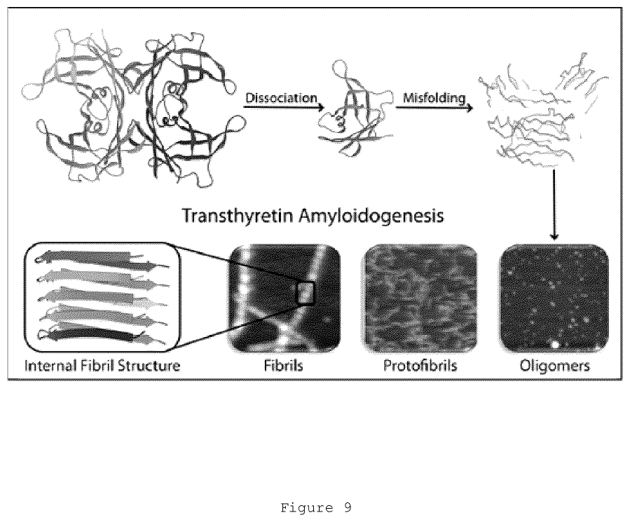

Transthyretin (TTR, thyroxine binding prealbumin) is a 55 kDa homotetramer comprised of four beta-sheet-rich, 127-residue polypeptide monomers that is largely synthesized in the liver for secretion into the blood. The circulating TTR molecule is a homotetramer formed by two dimers. To form the homotetrameric structure, two TTR monomers initially associate in a dimer subunit, which further associates with a second dimer subunit. The TTR dimer-dimer interface is relatively weak, and its dissociation is the rate-limiting step in the overall TTR tetramer dissociation process. The free dimer subunits may subsequently further dissociate into monomers that could potentially proceed to misfold and oligomerize (FIG. 9). Oligomerization can eventually lead to aggregation and formation of toxic amyloid fibrils, which underlies the pathophysiology of TTR amyloidosis (ATTR).

Autosomal dominant ATTR is a rare and progressive disease that involves severe organ damage due to the extracellular deposition of the aforementioned toxic TTR amyloid fibrils in tissues. The disease typically presents clinically as either TTR amyloid cardiomyopathy (ATTR-CM; which can lead to arrhythmias, arterial fibrillation, and biventricular heart failure) or as peripheral polyneuropathy (ATTR-PN; which can cause loss of sensation, tingling, numbness, or pain as well as damage to the autonomic nervous system) and can arise from pro-pathogenic monomers with inherited TTR mutations.

Non-hereditary ATTR emerges from wild-type TTR (WT-TTR) monomer misfolding in older individuals. Senile systemic amyloidosis (SSA), known currently as ATTRwt-CM, is a late-onset non-genetic disease associated with misaggregation of wild-type transthyretin (TTR) and accumulation of TTR amyloid deposits in extracellular compartments of tissues and organs throughout the body (Westermark, P. et al. 2003). The heart is usually the dominant site of involvement (Ruberg, F. L. & Berk, J. L. 2012; Ueda, M. et al. 2011). ATTRwt-CM affects approximately 25% of patients over the age of 80 (Hassan, W. et al. 2005). ATTRwt-CM is recognized as a major cause of severe cardiac dysfunction in the elderly, which includes congestive heart failure and cardiac death (Hassan, W. et al. 2005).

Currently available FDA-approved approaches for treating ATTR-CM and ATTR-PN include two treatments that reduce circulating TTR levels (the antisense oligonucleotide inotersen and the small interfering RNA (siRNA) patisiran) and the small molecule tafamidis (vyndaqel and vyndamax). Tafamidis and other small-molecule TTR-tetramer stabilizers bind to and stabilize circulating TTR tetramers. Ligand binding at the T4 sites has been shown to kinetically stabilize TTR tetramers by increasing the dissociative energy barrier of the native tetrameric state. This prevents TTR tetramer dissociation into dimers and monomers, thus inhibiting TTR amyloidosis cascade.

Tafamidis and patisiran are highly commercially successful treatments. However, elimination of TTR from circulation (patisiran) or using TTR ligands (Cioffi, C. L. et al. 2021) reduces circulating levels of serum RBP4. It seems that any kind of therapies that engage TTR or reduce its expression cause serum RBP4 reductions. Consistent with this conclusion, Alnylam Pharmaceuticals is advancing a next generation siRNA drug vutrisiran that reduces circulating TTR levels as a therapy for STGD1 (Alnylam 2021). The beneficial mechanism of the proposed use in Stargardt disease is serum RBP4 reduction.

ATTR therapies may cause ocular AEs similar to the ones described above for RBP4 antagonists. While the extent of ocular AEs for TTR therapies in the clinic is not well-documented in the literature, clinical use of the siRNA therapeutic patisiran (which induces TTR mRNA degradation by RNAi mechanism) was associated with symptoms of night blindness in a subset of patients treated for TTR amyloidosis (ONPATTRO® (patisiran) 2018).

Further, a disadvantage of RNA-targeting therapies is their partitioning exclusively to the liver upon systemic administration (Nogrady, B. 2019) (which does not prevent aggregation of extrahepatically expressed TTR) in addition to issues with inconvenient administration and sporadic toxicity of their formulations (Szebeni, J. 2014). Approximately 30% of patients do not respond to tafamidis (Monteiro, C. et al. 2019).

### 4. Comorbidity of Dry AMD and ATTRwt-CM

Senile systemic amyloidosis (SSA; also referred to as ATTRwt-CM) affects approximately 25% of patients over the age of 80 and is derived from aggregation of normal wild-type transthyretin (TTR) in various organs and tissues (Westermark, P. et al. 2003). This type of TTR amyloidosis mainly involves the heart and results in heart failure and/or atrial fibrillation, and may lead to death (Ruberg, F. L. & Berk, J. L. 2012). As illustrated in FIG. 9, in patients with SSA, a normally stable wild-type TTR tetramer may dissociate into monomers that can partially unfold and misassemble into amyloid fibrils forming pathogenic deposits in the heart causing amyloid cardiomyopathy. As 50% of plasma TTR is associated with retinol-RBP4 (Hyung, S. J. et al. 2010), formation of the tertiary retinol-RBP4-TTR complex is suggested to stabilize TTR tetramers and provide protection from formation of TTR amyloid fibrils (Hyung, S. J. et al. 2010; White, J. T. & Kelly, J. W. 2001). While TTR ligands from different structural classes are suggested to act as kinetic tetramer stabilizers capable of inhibiting TTR amyloid formation (Klabunde, T. et al. 2000; Miroy, G. J. et al. 1996; Penchala, S. C. et al. 2013; Petrassi, H. M. et al. 2005; Radovic, B. et al. 2006; Raghu, P. et al. 2002), there are currently no FDA-approved treatments for senile systemic amyloidosis.

Age-related macular degeneration (AMD) is the leading cause of blindness in developed countries. It is estimated that 62.9 million individuals worldwide currently have the most prevalent atrophic (dry) form of AMD; 8 million of them are Americans. Due to increasing life expectancy and current demographics, this number is expected to significantly increase in the future. With the possible exception of SYFOVRE™ mentioned above, which is a complement inhibitor indicated for the treatment of geographic atrophy (GA) secondary to age-related macular degeneration (AMD), there is currently no FDA-approved treatment for the dry form of AMD, which affects 90% of AMD patients.

Based on the high population frequency of senile systemic amyloidosis and dry AMD, significant comorbidity between the two conditions is expected.

### 5. Antibody-Drug Conjugate (ADC) Systems

An antibody-drug conjugate (ADC) is a class of therapeutics that combines the selectivity of monoclonal antibodies (mAbs) with the potency of cytotoxic drugs. It is designed to target specific cells, such as cancer cells, while minimizing the impact on healthy cells, thereby enhancing the efficacy and reducing the side effects of traditional chemotherapy (Su, Z. et al. 2021; Alas, M. et al. 2021; Tsuchikama, K. & An, Z. 2018).

The structure of an ADC typically consists of three main components:
Monoclonal Antibody (mAb): The mAb used in an ADC is designed to recognize and bind to a specific antigen that is overexpressed or selectively present on the target cells, such as tumor cells. The antibody provides the specificity and targeting capability of the ADC.
Linker: The linker serves as a bridge between the mAb and the cytotoxic drug. It is engineered to be stable in circulation but capable of releasing the drug payload selectively inside the target cells. The linker plays a crucial role in determining the release kinetics and stability of the ADC.
Cytotoxic Drug Payload: The cytotoxic drug, also known as the payload, is the pharmacologically active component of the ADC that exerts a toxic effect on the target cells. The drug is conjugated to the mAb via the linker. The choice of the cytotoxic drug depends on the therapeutic target and desired mechanism of action.

The mechanism of action of an ADC involves a series of steps:
Target Binding: The ADC is administered systemically, and the mAb component recognizes and binds specifically to the target antigen present on the surface of the cancer cells.
Internalization: Once the ADC binds to the target cells, it is internalized through receptor-mediated endocytosis, forming an endosome within the cell.
Intracellular Processing: Within the endosome, the linker is designed to be cleaved in response to specific conditions present in the target cell's environment, such as low pH or enzymatic activity. This cleavage releases the cytotoxic drug payload from the ADC.
Cytotoxic Effect: After release, the cytotoxic drug enters the cytoplasm or other cellular compartments, where it exerts its toxic effect. The drug may disrupt cellular processes, inhibit DNA replication, promote apoptosis (programmed cell death), or interfere with microtubule formation, depending on the specific drug used.

The goal of ADC therapy is to deliver the cytotoxic drug specifically to the target cells, reducing off-target effects and minimizing damage to healthy tissues. By leveraging the targeting capabilities of monoclonal antibodies, ADCs offer the potential for improved therapeutic efficacy with reduced systemic toxicity compared to conventional chemotherapy.

The co-drug designs of the present disclosure take advantage of a wide variety of established linkers used in antibody-drug conjugate (ADC) systems for rapid payload release via acidic pH or proteolytic cleavage (Su, Z. et al. 2021; Alas, M. et al. 2021; Tsuchikama, K. & An, Z. 2018). For example, a co-drug containing C20-D3-retinol linked to its core via a low-pH sensitive carbonate, silyl ether, or ester linker will rapidly cleave in the stomach and upper GI tract, readily releasing C20-D3-retinol for intestinal absorption and chylomicron packaging. Bispecific RBP4/TTR ligands of the co-drugs may be attached to their respective cores via ester, carbamate, or hemiaminal linkages, which will undergo rapid chemical and enzymatic hydrolysis in the GI tract.

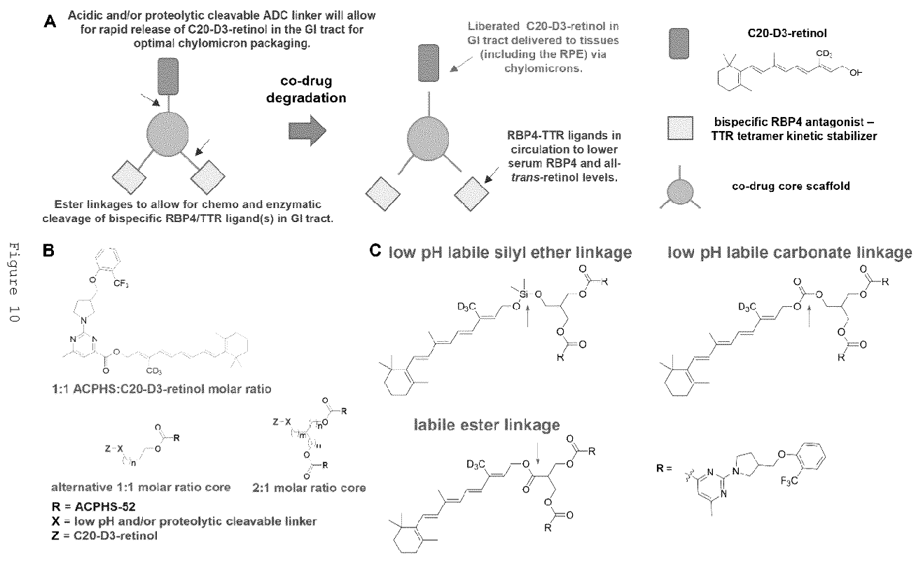

FIG. 10 describes key design principles of the co-drug platform of the present disclosure, using a bispecific RBP4/TTR ligand named ACPHS-52 as an example, which has the following chemical formula:

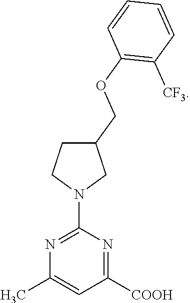 [SMILES: Cc1cc(C(=O)O)nc(N2CCC(COc3ccccc3C(F)(F)F)C2)n1]

Given that excessive amounts of dietary vitamin A may lead to liver damage (Park, J. et al. 2020; Nollevaux, M. C. et al. 2006), it is important to have an optimal ratio of a bispecific RBP4/TTR ligand to C20-D3-retinol in a co-drug to avoid delivering excessive retinol while providing an efficacious dose of the bispecific RBP4/TTR ligand. For efficacy, a 5-20 mg daily human dose of a bispecific RBP4/TTR ligand is required. On the other hand, daily consumption of up to 3 mg of retinol is safe.

One embodiment of the present disclosure is a co-drug that: contains an optimal molar ratio of a bispecific RBP4/TTR ligand to C20-D3-retinol (e.g., within a 1:1 to 5:1 range or within a 1:1 to 1:5 range); and can readily degrade to liberate the bispecific RBP4/TTR ligand and the C20-D3-retinol in the GI tract for chylomicron packaging and delivery. A wide variety of established linkers used in antibody-drug conjugate (ADC) systems for rapid payload release via acidic pH or proteolytic cleavage may be used for this purpose (Su, Z. et al. 2021; Alas, M. et al. 2021; Tsuchikama, K. & An, Z. 2018). For example, a co-drug containing C20-D3-retinol linked to a core via a carbonate or silyl ether linker can rapidly cleave in the stomach and upper GI tract, readily releasing C20-D3-retinol for intestinal absorption and chylomicron packaging. The bispecific RBP4/TTR ligands of the co-drugs can be attached to their respective cores via ester, carbamate, or hemiaminal linkages, which will undergo rapid chemo- and enzymatic hydrolysis in the GI tract.

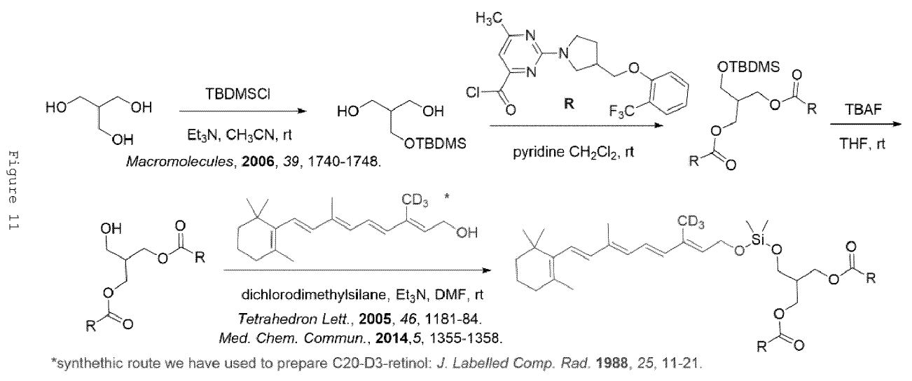

As the design of co-drugs requires that they rapidly degrade within the GI tract, one of ordinary skill in the art would know that the key parameters to be monitored include: (1) chemical stability time course under various pH ranges within a buffered aqueous environment and monitoring of rate of C20-D3-retinol release and bispecific RBP4/TTR ligand release; (2) aqueous solubility; and (3) careful monitoring of the oral absorption and PK of both payloads. These data may be used in iterative design cycles with various diverse core scaffolds and cleavable linkers to avoid PK variability. FIG. 11 shows an example of how a silyl-ether-containing co-drug can be prepared by using robust and well-precedented chemistry and by exploiting widely utilized ADC methodology for cleavable linkers.

A bispecific RBP4/TTR ligand and a C20-D3-visual-chromophore-producing compound may also be chemically bonded directly to each other to form a co-drug, namely, without involving an intervening linker. One such embodiment is shown in FIG. 10B as “1:1 ACPHS:C20-D3-retinol molar ratio.”

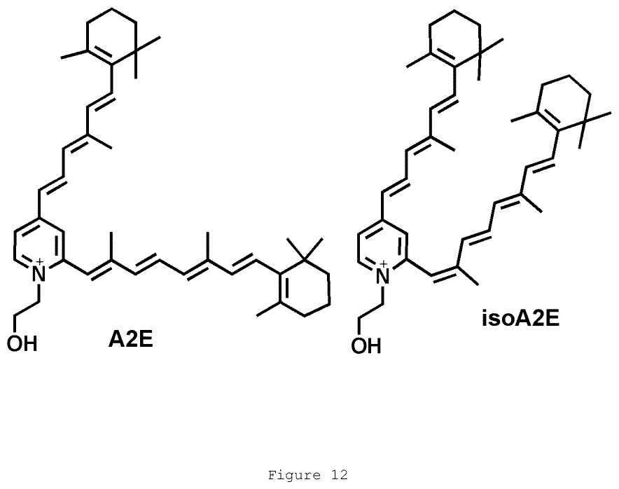

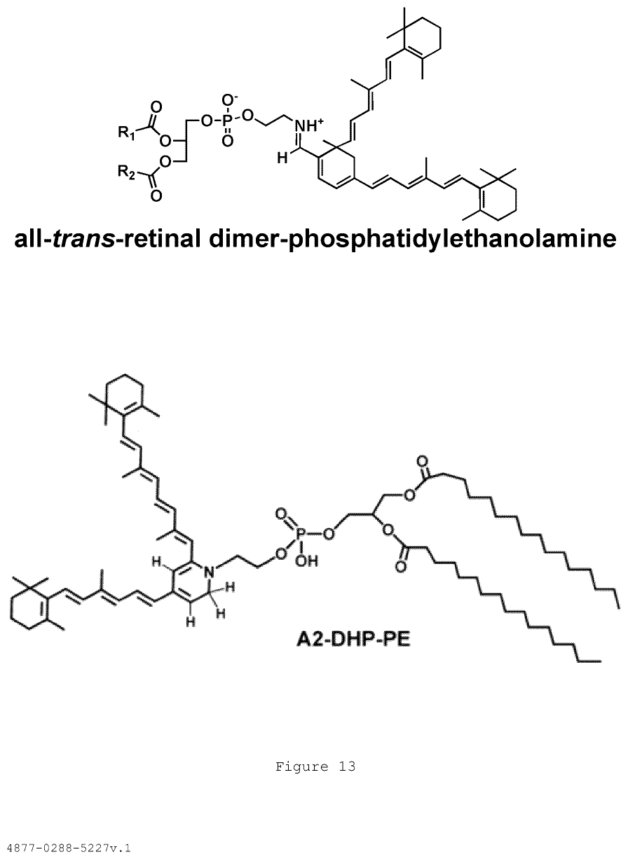

As used herein, “bisretinoid lipofuscin” is lipofuscin containing a cytotoxic bisretinoid. Cytotoxic bisretinoids include but are not necessarily limited to A2E, isoA2E, atRAL di-PE (all-trans-retinal dimer-phosphatidylethanolamine), and A2-DHP-PE (A2-dihydropyridine-phosphatidylethanolamine) (FIGS. 12 and 13). Bisretinoid-mediated macular degeneration may comprise the accumulation of lipofuscin deposits in the retinal pigment epithelium.

Transthyretin (TTR) amyloidosis (ATTR) is a neurodegenerative disease and includes, but is not limited to, senile systemic amyloidosis (SSA), peripheral polyneuropathy (ATTR-PN), or cardiomyopathy (ATTR-CM).

As used herein, “simultaneous administration” or “administering simultaneously” refers to administration of an admixture (whether a true mixture, a suspension, an emulsion, or other physical combination) of the first compound and the second compound. In this case, the combination may be the admixture or separate containers of the first compound and the second compound that are combined just prior to administration.

As used herein, “contemporaneous administration” or “administering contemporaneously” refers to the separate administration of the first compound and the second compound at the same time, or at times sufficiently close together that an additive or preferably synergistic activity relative to the activity of either the first compound or the second compound alone is observed.

As used herein, “concomitant administration” or “administering concomitantly” refers to the administration of two agents given in close enough temporal proximity to allow the individual therapeutic effects of each agent to overlap.

The present disclosure provides a compound having the general structure (P-)ₐM(-Q)_{b} or P(-Q)_{b}, wherein:
P represents a single-valence group formed by eliminating a hydrogen atom bonded to a heteroatom or by eliminating a hydroxyl group from a bispecific RBP4/TTR ligand;
a is 1, 2, 3, 4, or 5;
Q represents a single-valence group formed by eliminating a hydrogen atom bonded to a heteroatom or by eliminating a hydroxyl group from a C20-D3-visual-chromophore-producing compound;
b is 1, 2, 3, 4, or 5;
M represents an (a+b)-valence group comprising carbon, hydrogen, and oxygen atoms;
each P in (P-)ₐM(-Q)_{b} is independently bonded to M via an ester, carbamate, or hemiaminal linkage that is cleavable at an acidic pH or is enzymatically cleavable;
each Q in (P-)ₐM(-Q)_{b} is independently bonded to M via a carbonate, silyl ether, or ester linkage that is cleavable at an acidic pH or is enzymatically cleavable;
each Q in P(-Q)_{b} is independently bonded to P via an ester, carbamate, or hemiaminal linkage that is cleavable at an acidic pH or is enzymatically cleavable;
the bispecific RBP4/TTR ligand is a compound having the structure:

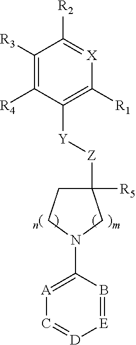

wherein
   X is CR₆ or N;
   R₁, R₂, R₃, R₄, and R₆ are each independently —H, —F, —Cl, —Br, —I, —NO₂, —CN, —CF₃, —CF₂H, —OCF₃, -(alkyl), -(haloalkyl), -(alkenyl), -(alkynyl), -(aryl), -(heteroaryl), -(cycloalkyl), -(cycloalkylalkyl), -(heteroalkyl), heterocycle, heterocycloalkyl, -(alkylheteroalkyl), -(alkylaryl), —OH, —OAc, —O-(alkyl), —O-(alkenyl), —O-(alkynyl), —O-(aryl), —O-(heteroaryl), —SH, —S-(alkyl), —S-(alkenyl), —S-(alkynyl), —S-(aryl), —S-(heteroaryl), —NH₂, —NH-(alkyl), —NH-(alkenyl), —NH-(alkynyl), —NH-(aryl), —NH-(heteroaryl), —C(O)R₇, —S(O)R₇, —SO₂R₇, —NHSO₂R₇, —OC(O)R₇, —SC(O)R₇, —NHC(O)R₈, or —NHC(S)R₈, wherein R₇ is H, -(alkyl), —OH, —O(alkyl), —NH₂, —NH(alkyl), or —N(alkyl)₂, and
wherein R₈ is -(alkyl), —O-(alkyl), —NH₂, —NH(alkyl), or —N(alkyl)₂;
   Y is O, S, N, NH, or a bond;
   Z is O, S, N, NH, (CH₂)ₒ, or a bond;
   R₅ is H, OH, halogen, or alkyl, or R₅ is (CH₂)ₚ and is bound to Y when Y is N to form a ring together with Z;
   o and p are independently 0, 1, 2, or 3;
   m and n are independently 0, 1, 2, 3, or 4;
   A, C, and D are each independently N or CR₉;
   R₉ is H, halogen, —OH, alkyl, cycloalkyl, cycloalkylalkyl, —O-(alkyl), —S-(alkyl), —NH₂, —NH(alkyl), —NH(alkyl)₂, —CO₂H, or —CO(O-alkyl);
   B and E are N, CR₉, or C—F-G wherein at least one of B or E is C—F-G;
   F is absent or present, and when present, is

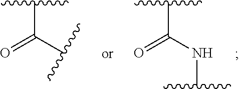

and
G is H, substituted or unsubstituted monocycle, bicycle, heteromonocycle, heterobicycle, aryl, heteroaryl, alkyl, cycloalkyl, cycloalkylalkyl, CO₂H, COOR₁₀, OH, OR₁₀, NH₂, NHR₁₀, NR₁₀R₁₁, SO₂ (alkyl), SO₂(cycloalkyl), SO₂(cycloalkylalkyl), CH₂NHR₁₀, CH₂NR₁₀R₁₁, or CH₂COOR₁₀,
wherein R₁₀ and R₁₁ are each independently H, alkyl, cycloalkyl, —C(O)-alkyl, —C(O)-cycloalkyl, —C(O)OH, —C(O)—O-alkyl, —C(O)—O-cycloalkyl, —C(O)NH₂, —C(O)NH(alkyl), —C(O)NH(cycloalkyl), —C(O)N(alkyl)₂, —CH₂NH (alkyl), —CH₂COOH, —SO₂CH₃, —OH, —O(alkyl), —NH₂, —NH(alkyl), or —N(alkyl)₂;
   or a pharmaceutically acceptable salt thereof; and
the C20-D3-visual-chromophore-producing compound is selected from the group consisting of C20-D3-retinol, C20-D3-9-cis-retinol, C20-D3-11-cis-retinol, and C20-D3-C20′-D3-β-carotene.

In some embodiments, the bispecific RBP4/TTR ligand is the compound wherein m is 1 or 2 and n is 0, 1, or 2.

In some embodiments, the bispecific RBP4/TTR ligand is the compound wherein m is 1 and n is 1.

In some embodiments, the bispecific RBP4/TTR ligand is the compound wherein Y and Z are each independently CH₂, O, S, or NH.

In some embodiments, the bispecific RBP4/TTR ligand is the compound wherein Y is O and Z is CH₂.

In some embodiments, the bispecific RBP4/TTR ligand is the compound wherein A and B are N, C and D are CR₉, and E is C—F-G.

In some embodiments, the bispecific RBP4/TTR ligand is the compound wherein A, B, C, and D are CR₉, and E is C—F-G.

In some embodiments, the bispecific RBP4/TTR ligand is the compound wherein A is N, B is C—F-G, and C, D, and E are each CR₉.

In some embodiments, the bispecific RBP4/TTR ligand is the compound wherein A is N, B, C, and D are each CR₉, and E is C—F-G.

In some embodiments, the bispecific RBP4/TTR ligand is the compound wherein A, C, and D are each CR₉, B is N, and E is C—F-G.

In some embodiments, the bispecific RBP4/TTR ligand is the compound wherein:
X is CR₆ or N; and
R₁, R₂, R₃, R₄, and R₆ are each independently H, t-butyl, cyclopentyl, cyclohexyl, CF₃, F, Cl, CN, or —OCH₃.

In some embodiments, the bispecific RBP4/TTR ligand is the compound wherein R₁ or R₄ is CF₃.

In some embodiments, the bispecific RBP4/TTR ligand is the compound wherein
X is CR₆,
R₁ is CF₃, and
R₂, R₃, R₄, and R₆ are each independently H, t-butyl,
cyclopentyl, cyclohexyl, CF₃, F, Cl, CN, or —OCH₃.

In some embodiments, the bispecific RBP4/TTR ligand is the compound having the structure:

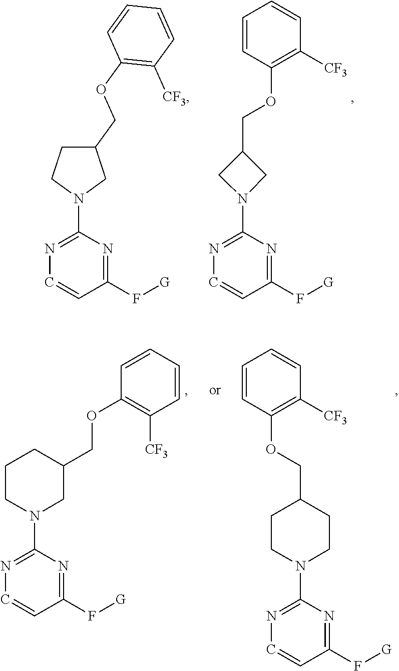

wherein
   C is CR₉;
   R₉ is H, halogen, —OH, alkyl, cycloalkyl, cycloalkylalkyl, —O-(alkyl), —S-(alkyl), —NH₂, —NH(alkyl), —NH(alkyl)₂, —CO₂H, or —CO(O-alkyl);
   F is absent or present, and when present, is

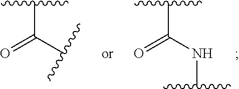

and
G is H, substituted or unsubstituted monocycle, bicycle, heteromonocycle, heterobicycle, aryl, heteroaryl, alkyl, cycloalkyl, cycloalkylalkyl, CO₂H, COOR₁₀, OH, OR₁₀, NH₂, NHR₁₀, NR₁₀R₁₁, SO₂(alkyl), SO₂(cycloalkyl), SO₂(cycloalkylalkyl), CH₂NHR₁₀, CH₂NR₁₀R₁₁, or CH₂COOR₁₀,
wherein R₁₀ and R₁₁ are each independently H, alkyl, cycloalkyl, —C(O)-alkyl, —C(O)-cycloalkyl, —C(O)OH, —C(O)—O-alkyl, —C(O)—O-cycloalkyl, —C(O)NH₂, —C(O)NH(alkyl), —C(O)NH(cycloalkyl), —C(O)N(alkyl)₂, —CH₂NH (alkyl), —CH₂COOH, —SO₂CH₃, —OH, —O(alkyl), —NH₂, —NH(alkyl), or —N(alkyl)₂;
   or a pharmaceutically acceptable salt thereof.

In some embodiments, the bispecific RBP4/TTR ligand is the compound wherein
C is CR₉ and
R₉ is H, alkyl, —O(alkyl), or —NH(alkyl).

In some embodiments, the bispecific RBP4/TTR ligand is the compound wherein R₉ is -alkyl.

In some embodiments, the bispecific RBP4/TTR ligand is the compound having the structure

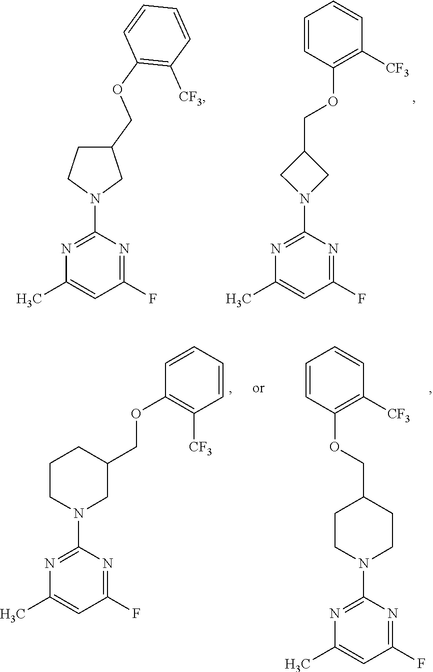

wherein F is a substituted or unsubstituted heteroaryl group.

In some embodiments, the bispecific RBP4/TTR ligand is the compound wherein F has the structure

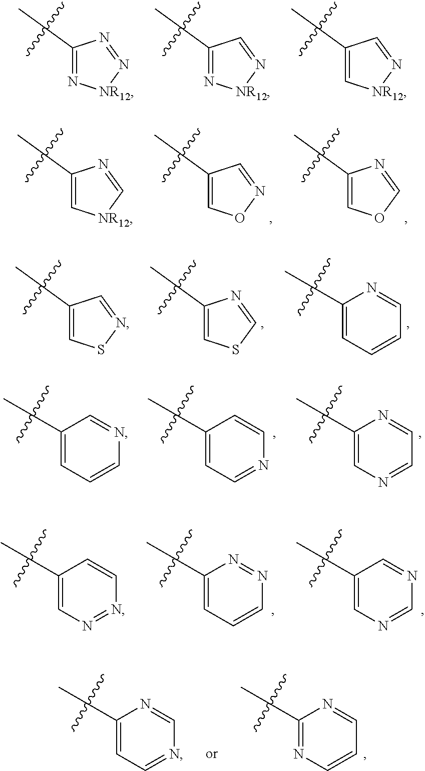

wherein R₁₂ is H, -(alkyl), -(alkenyl), or -(alkynyl).

In some embodiments, the bispecific RBP4/TTR ligand is the compound having the structure:

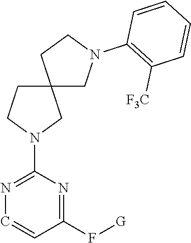

wherein
   C is CR₉;
   R₉ is H, halogen, —OH, alkyl, cycloalkyl, cycloalkylalkyl, —O-(alkyl), —S-(alkyl), —NH₂, —NH(alkyl), —NH(alkyl)₂, —CO₂H, or —CO(O-alkyl);
   F is absent or present, and when present, is

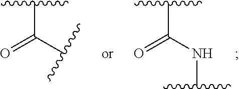

and
G is H, substituted or unsubstituted monocycle, bicycle, heteromonocycle, heterobicycle, aryl, heteroaryl, alkyl, cycloalkyl, cycloalkylalkyl, CO₂H, COOR₁₀, OH, OR₁₀, NH₂, NHR₁₀, NR₁₀R₁₁, SO₂ (alkyl), SO₂(cycloalkyl), SO₂(cycloalkylalkyl), CH₂NHR₁₀, CH₂NR₁₀R₁₁, or CH₂COOR₁₀,
wherein R₁₀ and R₁₁ are each independently H, alkyl, cycloalkyl, —C(O)-alkyl, —C(O)-cycloalkyl, —C(O)OH, —C(O)—O-alkyl, —C(O)—O-cycloalkyl, —C(O)NH₂, —C(O)NH(alkyl), —C(O)NH(cycloalkyl), —C(O)N(alkyl)₂, —CH₂NH (alkyl), —CH₂COOH, —SO₂CH₃, —OH, —O(alkyl), —NH₂, —NH(alkyl), or —N(alkyl)₂;
   or a pharmaceutically acceptable salt thereof.

In some embodiments, the bispecific RBP4/TTR ligand is the compound wherein
C is CR₉ and
R₉ is H, alkyl, —O(alkyl), or —NH(alkyl).

In some embodiments, the bispecific RBP4/TTR ligand is the compound wherein R₉ is -alkyl.

In some embodiments, the bispecific RBP4/TTR ligand is the compound having the structure

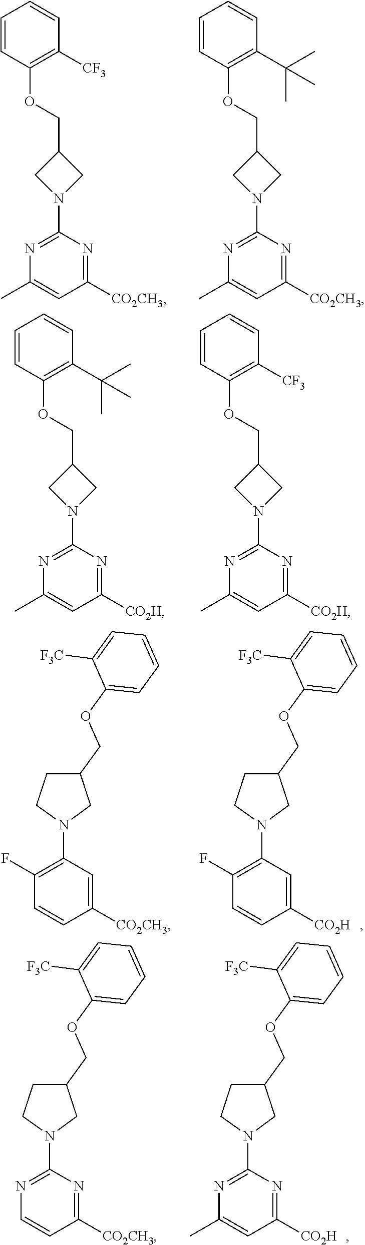

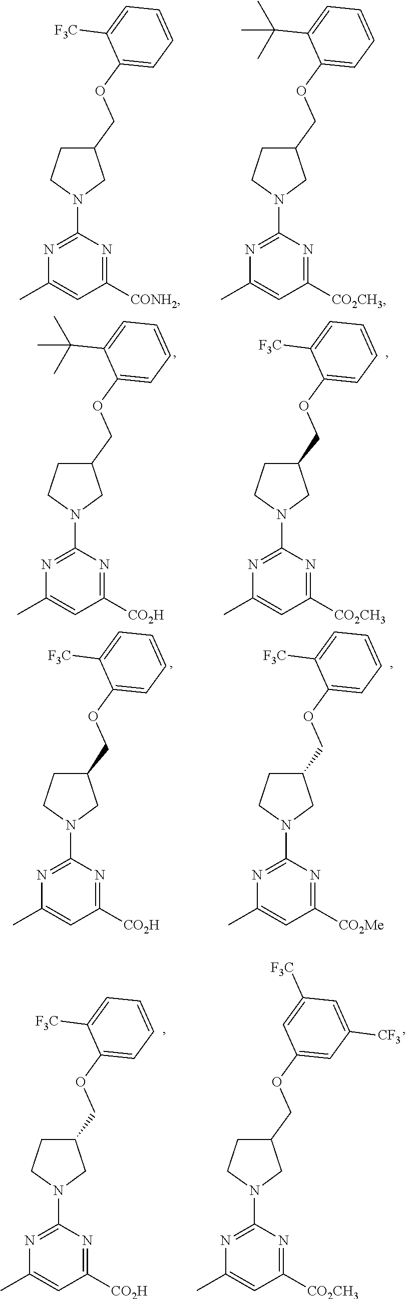

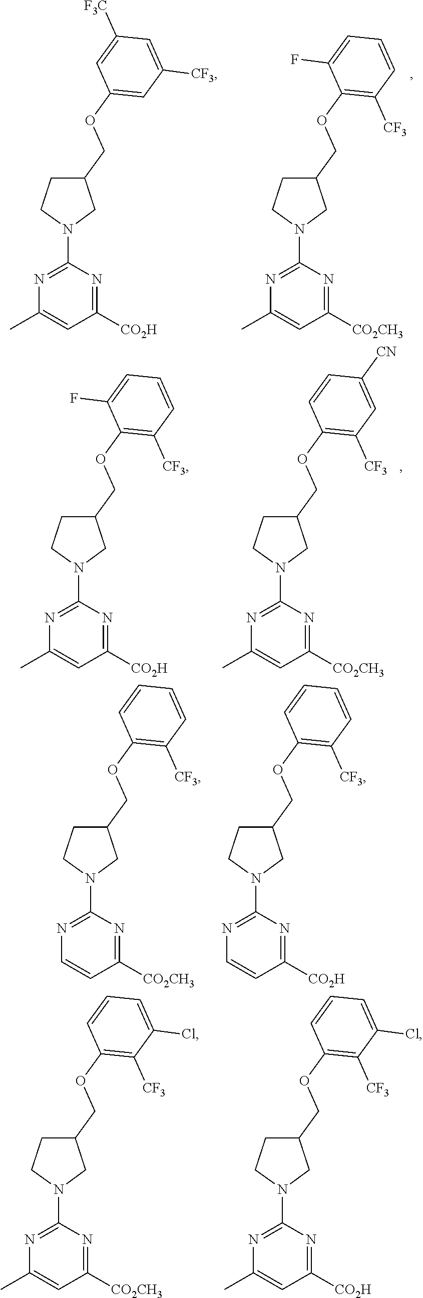

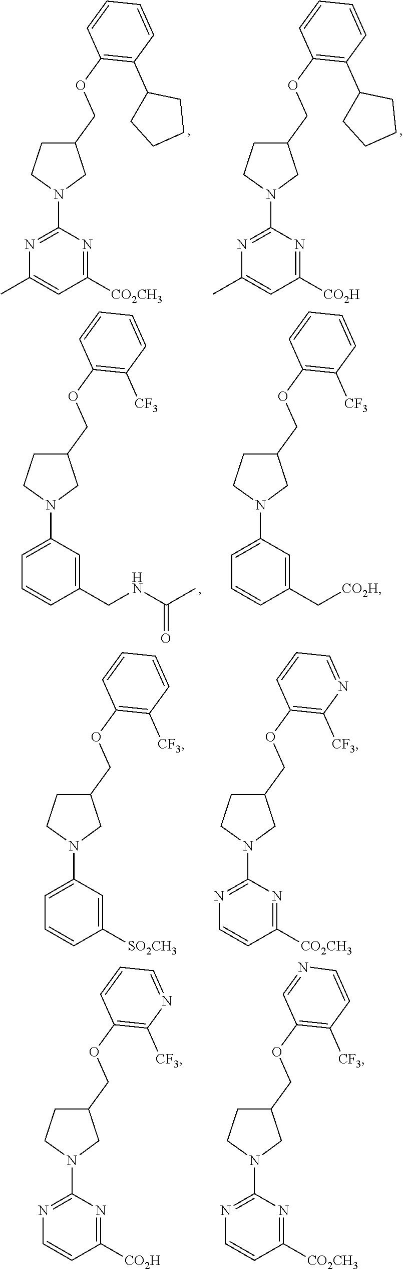

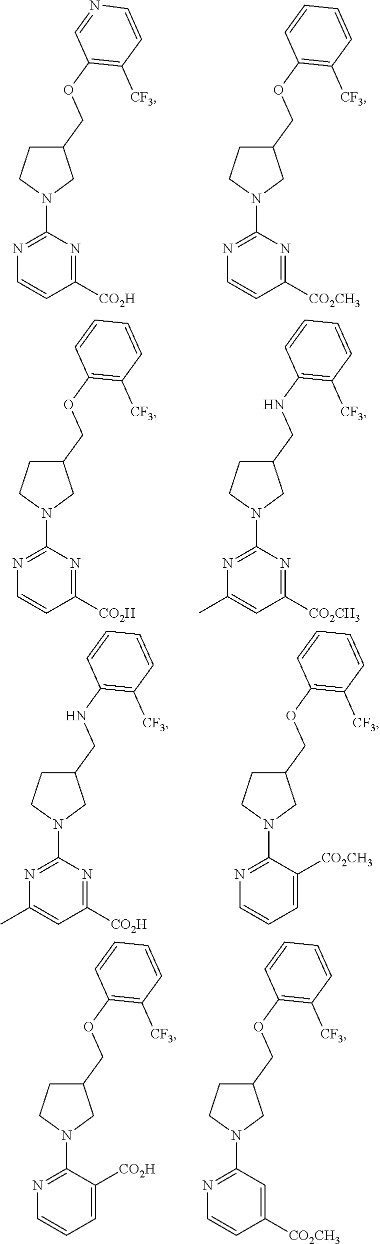

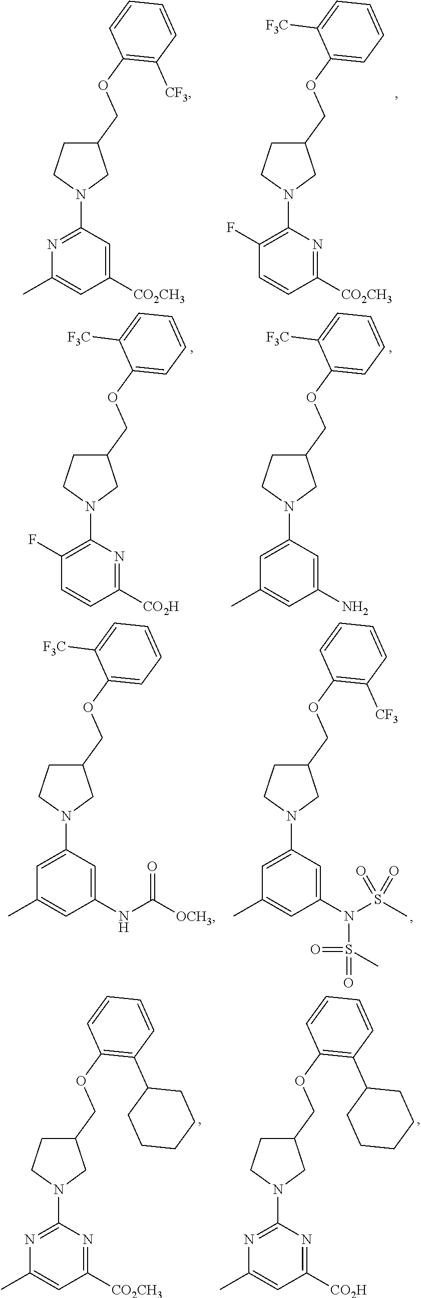

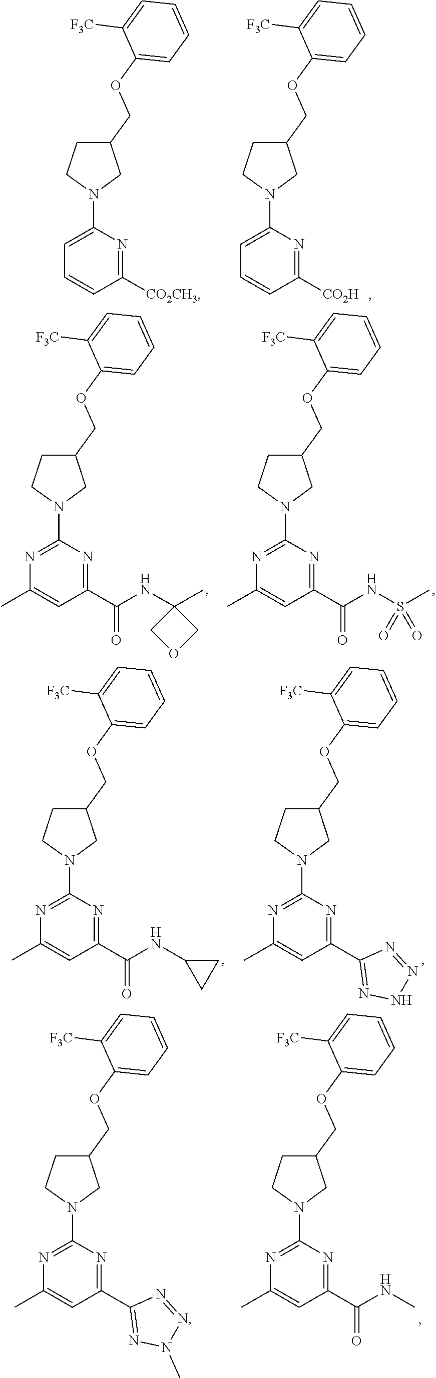

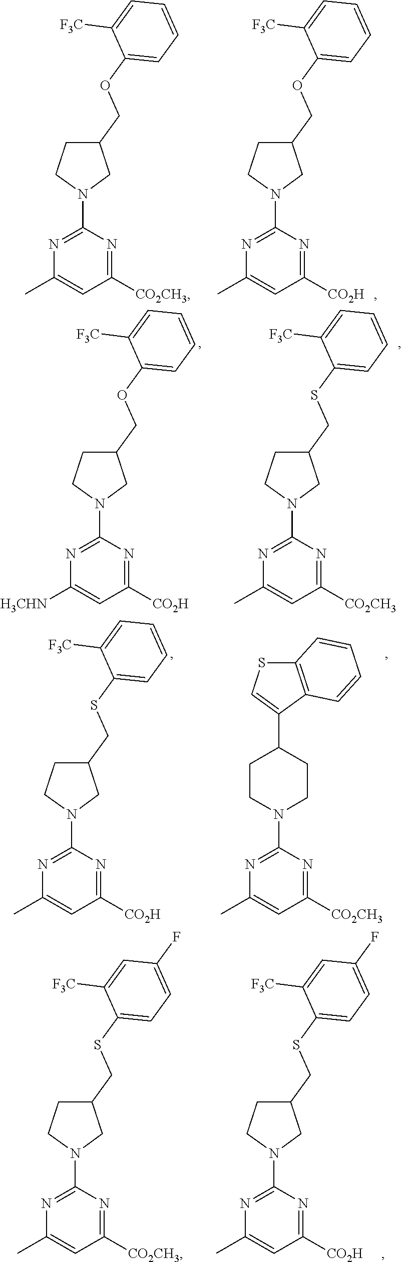

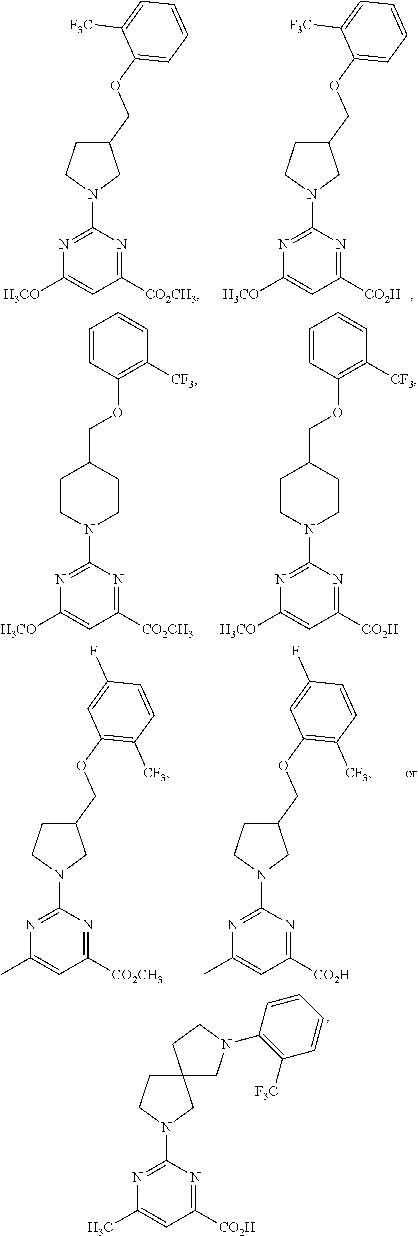

or a pharmaceutically acceptable salt of the compound.

In some embodiments, the bispecific RBP4/TTR ligand is the compound having the structure

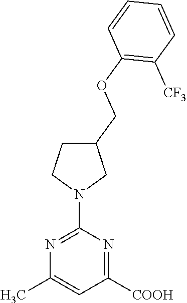 [SMILES: Cc1cc(C(=O)O)nc(N2CCC(COc3ccccc3C(F)(F)F)C2)n1]

or a pharmaceutically acceptable salt of the compound.

In some embodiments, the bispecific RBP4/TTR ligand is the compound having the structure

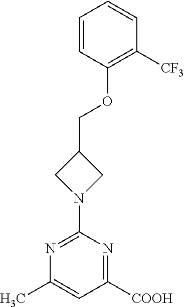 [SMILES: Cc1cc(C(=O)O)nc(N2CC(COc3ccccc3C(F)(F)F)C2)n1]

or a pharmaceutically acceptable salt of the compound.

In some embodiments, the bispecific RBP4/TTR ligand is the compound having the structure

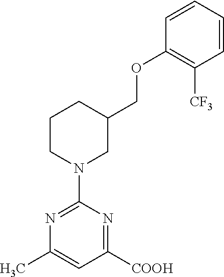 [SMILES: Cc1cc(C(=O)O)nc(N2CCCC(COc3ccccc3C(F)(F)F)C2)n1]

or a pharmaceutically acceptable salt of the compound.

In some embodiments, the bispecific RBP4/TTR ligand is the compound having the structure

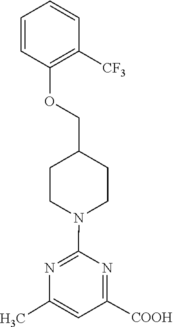 [SMILES: Cc1cc(C(=O)O)nc(N2CCC(COc3ccccc3C(F)(F)F)CC2)n1]

or a pharmaceutically acceptable salt of the compound.

In some embodiments, the bispecific RBP4/TTR ligand is the compound having the structure

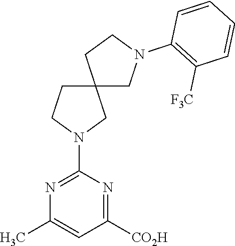 [SMILES: Cc1cc(C(=O)O)nc(N2CCC3(CCN(c4ccccc4C(F)(F)F)C3)C2)n1]

or a pharmaceutically acceptable salt of the compound.

In some embodiments, a is 2 and b is 1.

In some embodiments, a is 1 and b is 1.

In some embodiments, a is 2, b is 1, and M is

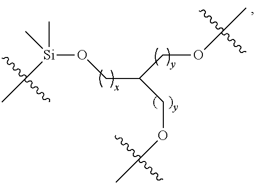

wherein x is 1, 2, 3, 4, or 5, and y is 1, 2, 3, 4, or 5.

In some embodiments, a is 2, b is 1, and M is

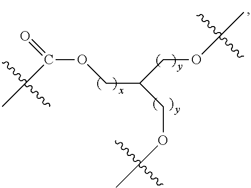

wherein x is 1, 2, 3, 4, or 5, and y is 1, 2, 3, 4, or 5.

In some embodiments, a is 2, b is 1, and M is

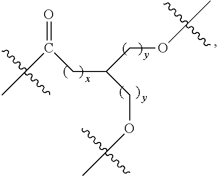

wherein x is 0, 1, 2, 3, or 4, and y is 1, 2, 3, 4, or 5.

In some embodiments, a is 1, b is 1, and M is

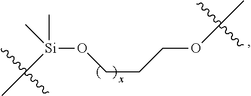

wherein x is 1, 2, 3, 4, or 5.

In some embodiments, a is 1, b is 1, and M is

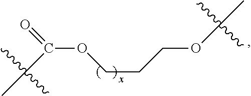

wherein x is 1, 2, 3, 4, or 5.

In some embodiments, a is 1, b is 1, and M is

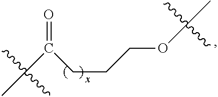

wherein x is 0, 1, 2, 3, or 4.

In some embodiments, P is

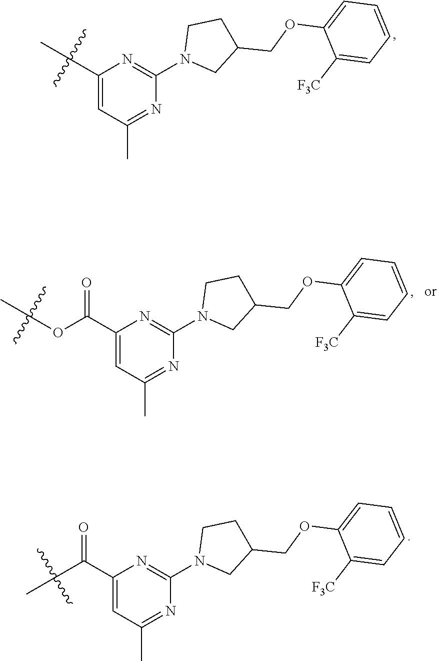 [SMILES: *c1cc(C)nc(N2CCC(COc3ccccc3C(F)(F)F)C2)n1 ; *OC(=O)c1cc(C)nc(N2CCC(COc3ccccc3C(F)(F)F)C2)n1 ; *C(=O)c1cc(C)nc(N2CCC(COc3ccccc3C(F)(F)F)C2)n1]

In some embodiments, Q is

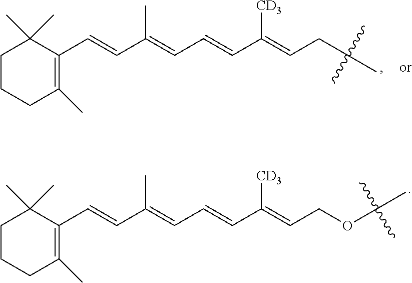 [SMILES: *C/C=C(/C=C/C=C(C)/C=C/C1=C(C)CCCC1(C)C)C([2H])([2H])[2H] ; *OC/C=C(/C=C/C=C(C)/C=C/C1=C(C)CCCC1(C)C)C([2H])([2H])[2H]]

In some embodiments, M is

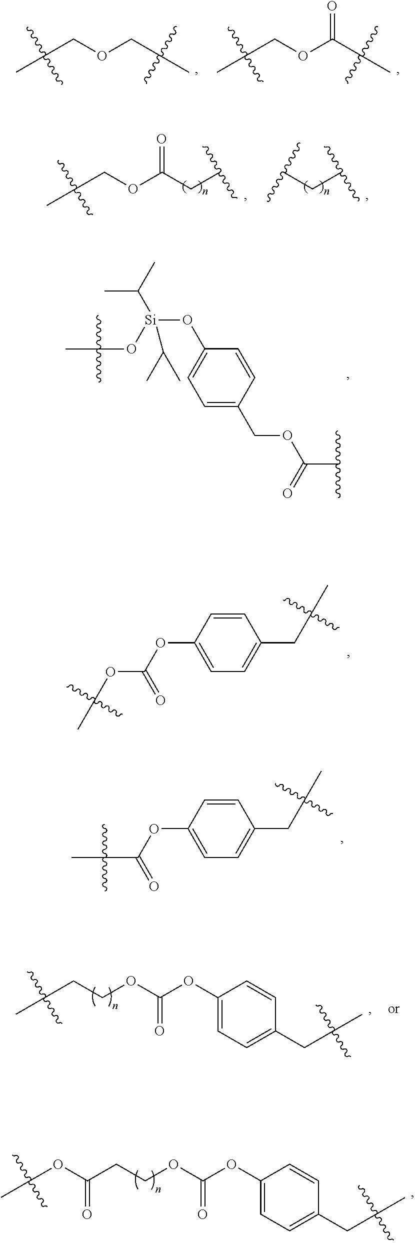

wherein n is 0-20.

In some embodiments, the present invention provides a compound having the following structure:

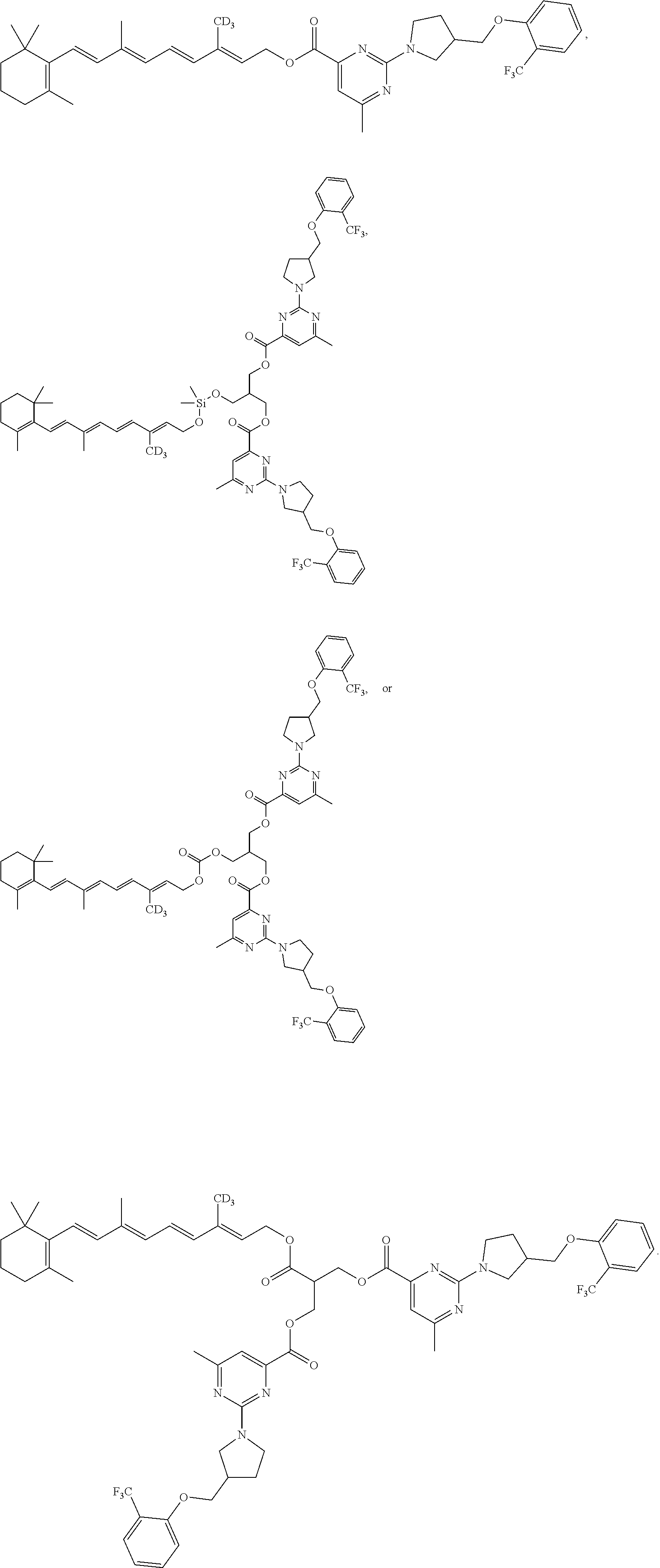

The present disclosure provides a pharmaceutical composition comprising the compound (P-)ₐM(-Q)_{b} or P(-Q)_{b} of the present disclosure and a pharmaceutically acceptable carrier.

The present disclosure provides a method for stabilizing TTR tetramers and for promoting rhodopsin and cone opsins production in a mammal, comprising administering to the mammal an effective amount of the compound of the present disclosure or an effective amount of the pharmaceutical composition of the present disclosure to effectively stabilize TTR tetramers and to promote rhodopsin and cone opsins production in the mammal.

The present disclosure provides a method for treating a disease characterized by excessive or age-related lipofuscin accumulation in the retina, or a TTR amyloidosis (ATTR) disease, or both a disease characterized by excessive or age-related lipofuscin accumulation and a TTR amyloidosis (ATTR) disease, in a mammal afflicted therewith, and for promoting rhodopsin and cone opsins production, comprising administering to the mammal an effective amount of the compound of the present disclosure or an effective amount of the pharmaceutical composition of the present disclosure.

In some embodiments of the method, the disease is further characterized by bisretinoid-mediated macular degeneration.

In some embodiments of the method, the amount of the compound is effective to lower the serum concentration of RBP4 in the mammal, or the amount of the compound is effective to lower the retinal concentration of a bisretinoid in lipofuscin in the mammal.

In some embodiments of the method, the amount of the compound is effective to stabilize TTR tetramers in the mammal.

In some embodiments of the method, the bisretinoid is A2E.

In some embodiments of the method, the bisretinoid is isoA2E.

In some embodiments of the method, the bisretinoid is A2-DHP-PE.

In some embodiments of the method, the bisretinoid is atRAL di-PE.

In some embodiments of the method, the disease characterized by age-related lipofuscin accumulation in the retina is Age-Related Macular Degeneration.

In some embodiments of the method, the disease characterized by age-related lipofuscin accumulation in the retina is dry (atrophic) Age-Related Macular Degeneration.

In some embodiments of the method, the disease characterized by excessive lipofuscin accumulation in the retina is Stargardt Disease.

In some embodiments of the method, the disease characterized by excessive lipofuscin accumulation in the retina is other forms of retinopathy caused by or associated with mutations in the ABCA4 gene, such as retinitis pigmentosa (RP19) or cone-rod dystrophy (CORD3).

In some embodiments of the method, the disease characterized by excessive lipofuscin accumulation in the retina is Best disease.

In some embodiments of the method, the disease characterized by excessive lipofuscin accumulation in the retina is adult vitelliform maculopathy.

In some embodiments of the method, the disease characterized by excessive lipofuscin accumulation in the retina is Stargardt-like macular dystrophy.

In some embodiments of the method, the administration is effective to reduce photoreceptor degeneration.

In some embodiments of the method, the method is further effective to stabilize TTR tetramers in the mammal.

In some embodiments of the method, the mammal is further afflicted with a TTR amyloidosis (ATTR) disease and the method is effective for treating the TTR amyloidosis (ATTR) disease in the mammal.

In some embodiments of the method, the TTR amyloidosis (ATTR) disease is senile systemic amyloidosis (SSA).

In some embodiments of the method, the TTR amyloidosis (ATTR) disease is peripheral polyneuropathy (ATTR-PN).

In some embodiments of the method, the TTR amyloidosis (ATTR) disease is cardiomyopathy (ATTR-CM).

In some embodiments of the method, the TTR amyloidosis (ATTR) disease is characterized by deposition of amyloid aggregates.

In some embodiments, the bisretinoid-mediated macular degeneration is Age-Related Macular Degeneration or Stargardt Disease.

In some embodiments, the bisretinoid-mediated macular degeneration is Age-Related Macular Degeneration.

In some embodiments, the bisretinoid-mediated macular degeneration is dry (atrophic) Age-Related Macular Degeneration.

In some embodiments, the bisretinoid-mediated macular degeneration is Stargardt Disease.

In some embodiments, the bisretinoid-mediated macular degeneration is other forms of retinopathy caused by or associated with mutations in the ABCA4 gene, such as retinitis pigmentosa (RP19) or cone-rod dystrophy (CORD3).

In some embodiments, the bisretinoid-mediated macular degeneration is Best disease.

In some embodiments, the bisretinoid-mediated macular degeneration is adult vitelliform maculopathy.

In some embodiments, the bisretinoid-mediated macular degeneration is Stargardt-like macular dystrophy.

In some embodiments, TTR amyloidosis (ATTR) diseases are characterized by the deposition of amyloid aggregates.

In some embodiments, TTR amyloidosis (ATTR) diseases are characterized by the deposition of amyloid aggregates derived from either mutant (TTRm) or wild-type (TTRwt).

In some embodiments, TTR amyloidosis (ATTR) disease is senile systemic amyloidosis (SSA).

In some embodiments, TTR amyloidosis (ATTR) disease is peripheral polyneuropathy (ATTR-PN).

In some embodiments, TTR amyloidosis (ATTR) disease is cardiomyopathy (ATTR-CM).

In some embodiments, the compound of the present disclosure, upon hydrolysis and/or proteolytic cleavage, exhibits dual retinol-binding protein 4 (RBP4) antagonist and transthyretin (TTR) tetramer kinetic stabilization activity as well as activity of promoting rhodopsin and cone opsins production.

In some embodiments, the compound of the present disclosure, upon hydrolysis and/or proteolytic cleavage, exhibits retinol-binding protein 4 (RBP4) antagonist activity as well as activity of promoting rhodopsin and cone opsins production.

In some embodiments, the compound of the present disclosure, upon hydrolysis and/or proteolytic cleavage, exhibits transthyretin (TTR) tetramer kinetic stabilization activity as well as activity of promoting rhodopsin and cone opsins production.

In some embodiments, the compound of the present disclosure, upon hydrolysis and/or proteolytic cleavage, reduces circulating RBP4 levels while simultaneously stabilizing unliganded TTR tetramers released from the holo-RBP4-TTR complex, and promotes rhodopsin and cone opsins production.

In some embodiments, the compound of the present disclosure, upon hydrolysis and/or proteolytic cleavage, reduces circulating RBP4 levels and promotes rhodopsin and cone opsins production.

In some embodiments, the compound of the present disclosure, upon hydrolysis and/or proteolytic cleavage, stabilizes unliganded TTR tetramers released from the holo-RBP4-TTR complex and promotes rhodopsin and cone opsins production.

In some embodiments, the compound of the present disclosure or the pharmaceutical composition of the present disclosure may be used for the treatment of dry age-related macular degeneration (AMD) and TTR amyloidosis (ATTR) comorbidities.

In some embodiments, the compound of the present disclosure or the pharmaceutical composition of the present disclosure may be used for the treatment of dry age-related macular degeneration (AND) and senile systemic amyloidosis (SSA).

In some embodiments, the compound of the present disclosure or the pharmaceutical composition of the present disclosure may be used for the treatment of dry age-related macular degeneration (AND) and peripheral polyneuropathy (ATTR-PN).

In some embodiments, the compound of the present disclosure or the pharmaceutical composition of the present disclosure may be used for the treatment of dry age-related macular degeneration (AND) and cardiomyopathy (ATTR-CM).

In some embodiments, the compound of the present disclosure or the pharmaceutical composition of the present disclosure may be used for the treatment of type 2 diabetes.

In some embodiments, the compound of the present disclosure or the pharmaceutical composition of the present disclosure may be used for the treatment of obesity.

In some embodiments, the compound of the present disclosure or the pharmaceutical composition of the present disclosure may be used for the treatment of insulin resistance.

In some embodiments, the compound of the present disclosure or the pharmaceutical composition of the present disclosure may be used for the treatment of cardiovascular disease.

In some embodiments, the compound of the present disclosure or the pharmaceutical composition of the present disclosure may be used for the treatment of hepatic steatosis.

In some embodiments, the compound of the present disclosure or the pharmaceutical composition of the present disclosure may be used for the treatment of non-alcoholic fatty liver disease (NAFLD).

In some embodiments of the method, the administration is oral.

In some embodiments, the mammal is a human.

The present disclosure provides a method for producing a compound having the structure (P-)₂M-Q wherein:
P represents a single-valence group formed by eliminating a hydroxyl group from a bispecific RBP4/TTR ligand;
Q represents a single-valence group formed by eliminating a hydrogen atom bonded to a heteroatom from a C20-D3-visual-chromophore-producing compound;
M represents a triple-valence group that is

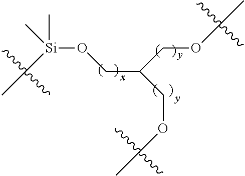

wherein x is 1 and y is 1;
each P is bonded to M via an ester linkage that is cleavable at an acidic pH or is enzymatically cleavable;
Q is bonded to M via a silyl ether linkage that is cleavable at an acidic pH or is enzymatically cleavable;
the bispecific RBP4/TTR ligand is a compound having the structure:

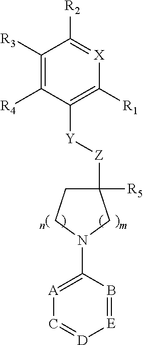

wherein
   X is CR₆ or N;
   R₁, R₂, R₃, R₄, and R₆ are each independently —H, —F, —Cl, —Br, —I, —NO₂, —CN, —CF₃, —CF₂H, —OCF₃, -(alkyl), -(haloalkyl), -(alkenyl), -(alkynyl), -(aryl), -(heteroaryl), -(cycloalkyl), -(cycloalkylalkyl), -(heteroalkyl), heterocycle, heterocycloalkyl, -(alkylheteroalkyl), -(alkylaryl), —OH, —OAc, —O-(alkyl), —O-(alkenyl), —O-(alkynyl), —O-(aryl), —O-(heteroaryl), —SH, —S-(alkyl), —S-(alkenyl), —S-(alkynyl), —S-(aryl), —S-(heteroaryl), —NH₂, —NH-(alkyl), —NH-(alkenyl), —NH-(alkynyl), —NH-(aryl), —NH-(heteroaryl), —C(O)R₇, —S(O)R₇, —SO₂R₇, —NHSO₂R₇, —OC(O)R₇, —SC(O)R₇, —NHC(O)R₈, or —NHC(S)R₈, wherein R₇ is H, -(alkyl), —OH, —O(alkyl), —NH₂, —NH(alkyl), or —N(alkyl)₂, and
wherein R₈ is -(alkyl), —O-(alkyl), —NH₂, —NH(alkyl), or —N(alkyl)₂;
   Y is O, S, N, NH, or a bond;
   Z is O, S, N, NH, (CH₂)ₒ, or a bond;
   R₅ is H, OH, halogen, or alkyl, or R₅ is (CH₂)ₚ and is bound to Y when Y is N to form a ring together with Z;
   o and p are independently 0, 1, 2, or 3;
   m and n are independently 0, 1, 2, 3, or 4;
   A, C, and D are each independently N or CR₉;
   R₉ is H, halogen, —OH, alkyl, cycloalkyl, cycloalkylalkyl, —O-(alkyl), —S-(alkyl), —NH₂, —NH(alkyl), —NH(alkyl)₂, —CO₂H, or —CO(O-alkyl);
   B is N, CR₉, or C—F-G;
   F is absent or present, and when present, is

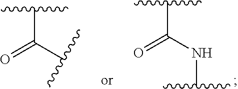

G is H, substituted or unsubstituted monocycle, bicycle, heteromonocycle, heterobicycle, aryl, heteroaryl, alkyl, cycloalkyl, cycloalkylalkyl, CO₂H, COOR₁₀, OH, OR₁₀, NH₂, NHR₁₀, NR₁₀R₁₁, SO₂ (alkyl), SO₂(cycloalkyl), SO₂(cycloalkylalkyl), CH₂NHR₁₀, CH₂NR₁₀R₁₁, or CH₂COOR₁₀,
wherein R₁₀ and R₁₁ are each independently H, alkyl, cycloalkyl, —C(O)-alkyl, —C(O)-cycloalkyl, —C(O)OH, —C(O)—O-alkyl, —C(O)—O-cycloalkyl, —C(O)NH₂, —C(O)NH(alkyl), —C(O)NH(cycloalkyl), —C(O)N(alkyl)₂, —CH₂NH (alkyl), —CH₂COOH, —SO₂CH₃, —OH, —O(alkyl), —NH₂, —NH(alkyl), or —N(alkyl)₂; and
   E is C—F—CO₂H;
      or a pharmaceutically acceptable salt thereof; and
the C20-D3-visual-chromophore-producing compound is selected from the group consisting of C20-D3-retinol, C20-D3-9-cis-retinol, and C20-D3-11-cis-retinol;
   the method comprising the steps of:
modifying one of three hydroxyl groups of 2-(hydroxymethyl)-1,3-propanediol by a protecting group;
performing a dehydration reaction between the protecting-group-modified 2-(hydroxymethyl)-1,3-propanediol and the bispecific RBP4/TTR ligand to form a diester product;
removing the protecting group from the diester product; and
reacting the deprotected diester product with dichlorodimethylsilane and the C20-D3-visual-chromophore-producing compound to form the compound (P-)₂M-Q.

The present disclosure provides a method for producing a compound having the structure (P-)₂M-Q wherein:
P represents a single-valence group formed by eliminating a hydroxyl group from a bispecific RBP4/TTR ligand;
Q represents a single-valence group formed by eliminating a hydrogen atom bonded to a heteroatom from a C20-D3-visual-chromophore-producing compound;
M represents a triple-valence group that is

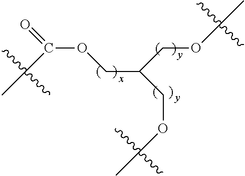

wherein x is 1 and y is 1;
each P is bonded to M via an ester linkage that is cleavable at an acidic pH or is enzymatically cleavable;
Q is bonded to M via a carbonate linkage that is cleavable at an acidic pH or is enzymatically cleavable;
the bispecific RBP4/TTR ligand is a compound having the structure:

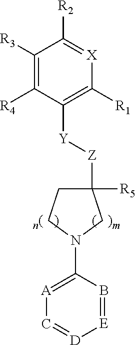

wherein
   X is CR₆ or N;
   R₁, R₂, R₃, R₄, and R₆ are each independently —H, —F, —Cl, —Br, —I, —NO₂, —CN, —CF₃, —CF₂H, —OCF₃, -(alkyl), -(haloalkyl), -(alkenyl), -(alkynyl), -(aryl), -(heteroaryl), -(cycloalkyl), -(cycloalkylalkyl), -(heteroalkyl), heterocycle, heterocycloalkyl, -(alkylheteroalkyl), -(alkylaryl), —OH, —OAc, —O-(alkyl), —O-(alkenyl), —O-(alkynyl), —O-(aryl), —O-(heteroaryl), —SH, —S-(alkyl), —S-(alkenyl), —S-(alkynyl), —S-(aryl), —S-(heteroaryl), —NH₂, —NH-(alkyl), —NH-(alkenyl), —NH-(alkynyl), —NH-(aryl), —NH-(heteroaryl), —C(O)R₇, —S(O)R₇, —SO₂R₇, —NHSO₂R₇, —OC(O)R₇, —SC(O)R₇, —NHC(O)R₈, or —NHC(S)R₈, wherein R₇ is H, -(alkyl), —OH, —O(alkyl), —NH₂, —NH(alkyl), or —N(alkyl)₂, and
wherein R₈ is -(alkyl), —O-(alkyl), —NH₂, —NH(alkyl), or —N(alkyl)₂;
   Y is O, S, N, NH, or a bond;
   Z is O, S, N, NH, (CH₂)ₒ, or a bond;
   R₅ is H, OH, halogen, or alkyl, or R₅ is (CH₂)ₚ and is bound to Y when Y is N to form a ring together with Z;
   o and p are independently 0, 1, 2, or 3;
   m and n are independently 0, 1, 2, 3, or 4;
   A, C, and D are each independently N or CR₉;
   R₉ is H, halogen, —OH, alkyl, cycloalkyl, cycloalkylalkyl, —O-(alkyl), —S-(alkyl), —NH₂, —NH(alkyl), —NH(alkyl)₂, —CO₂H, or —CO(O-alkyl);
   B is N, CR₉, or C—F-G;
   F is absent or present, and when present, is

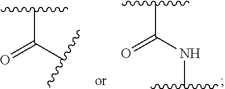

G is H, substituted or unsubstituted monocycle, bicycle, heteromonocycle, heterobicycle, aryl, heteroaryl, alkyl, cycloalkyl, cycloalkylalkyl, CO₂H, COOR₁₀, OH, OR₁₀, NH₂, NHR₁₀, NR₁₀R₁₁, SO₂ (alkyl), SO₂(cycloalkyl), SO₂(cycloalkylalkyl), CH₂NHR₁₀, CH₂NR₁₀R₁₁, or CH₂COOR₁₀,
wherein R₁₀ and R₁₁ are each independently H, alkyl, cycloalkyl, —C(O)-alkyl, —C(O)-cycloalkyl, —C(O)OH, —C(O)—O-alkyl, —C(O)—O-cycloalkyl, —C(O)NH₂, —C(O)NH(alkyl), —C(O)NH(cycloalkyl), —C(O)N(alkyl)₂, —CH₂NH (alkyl), —CH₂COOH, —SO₂CH₃, —OH, —O(alkyl), —NH₂, —NH(alkyl), or —N(alkyl)₂; and
   E is C—F—CO₂H;
      or a pharmaceutically acceptable salt thereof; and
the C20-D3-visual-chromophore-producing compound is selected from the group consisting of C20-D3-retinol, C20-D3-9-cis-retinol, and C20-D3-11-cis-retinol;
   the method comprising the steps of:
performing a dehydration reaction between 2-(benzyloxymethyl)-1,3-propanediol and the bispecific RBP4/TTR ligand or a derivative thereof modified by protecting groups, to form a diester product;
reacting the diester product with the C20-D3-visual-chromophore-producing compound to form a mono-carbonate-linked diester product; and
removing the protecting groups, if present, from the mono-carbonate-linked diester product;
to form the compound (P-)₂M-Q.

In some embodiments, the C20-D3-visual-chromophore-producing compound is selected from the group consisting of C20-D3-retinol, C20-D3-9-cis-retinol, and C20-D3-11-cis-retinol.

The present disclosure provides a pharmaceutical composition comprising a bispecific RBP4/TTR ligand, a C20-D3-visual-chromophore-producing compound, and a pharmaceutically acceptable carrier, wherein:
the bispecific RBP4/TTR ligand is a compound having the structure:

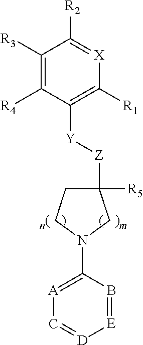

wherein
   X is CR₆ or N;
   R₁, R₂, R₃, R₄, and R₆ are each independently —H, —F, —Cl, —Br, —I, —NO₂, —CN, —CF₃, —CF₂H, —OCF₃, -(alkyl), -(haloalkyl), -(alkenyl), -(alkynyl), -(aryl), -(heteroaryl), -(cycloalkyl), -(cycloalkylalkyl), -(heteroalkyl), heterocycle, heterocycloalkyl, -(alkylheteroalkyl), -(alkylaryl), —OH, —OAc, —O-(alkyl), —O-(alkenyl), —O-(alkynyl), —O-(aryl), —O-(heteroaryl), —SH, —S-(alkyl), —S-(alkenyl), —S-(alkynyl), —S-(aryl), —S-(heteroaryl), —NH₂, —NH-(alkyl), —NH-(alkenyl), —NH-(alkynyl), —NH-(aryl), —NH-(heteroaryl), —C(O)R₇, —S(O)R₇, —SO₂R₇, —NHSO₂R₇, —OC(O)R₇, —SC(O)R₇, —NHC(O)R₈, or —NHC(S)R₈,
wherein R₇ is H, -(alkyl), —OH, —O(alkyl), —NH₂, —NH(alkyl), or —N(alkyl)₂, and wherein R₈ is -(alkyl), —O-(alkyl), —NH₂, —NH(alkyl), or —N(alkyl)₂;
   Y is O, S, N, NH, or a bond;
   Z is O, S, N, NH, (CH₂)ₒ, or a bond;
   R₅ is H, OH, halogen, or alkyl, or R₅ is (CH₂)ₚ and is bound to Y when Y is N to form a ring together with Z;
   o and p are independently 0, 1, 2, or 3;
   m and n are independently 0, 1, 2, 3, or 4;
   A, C, and D are each independently N or CR₉;
   R₉ is H, halogen, —OH, alkyl, cycloalkyl, cycloalkylalkyl, —O-(alkyl), —S-(alkyl), —NH₂, —NH(alkyl), —NH(alkyl)₂, —CO₂H, or —CO(O-alkyl);
   B and E are N, CR₉, or C—F-G wherein at least one of B or E is C—F-G;
   F is absent or present, and when present, is

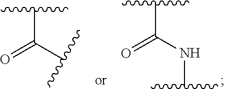

and
G is H, substituted or unsubstituted monocycle, bicycle, heteromonocycle, heterobicycle, aryl, heteroaryl, alkyl, cycloalkyl, cycloalkylalkyl, CO₂H, COOR₁₀, OH, OR₁₀, NH₂, NHR₁₀, NR₁₀R₁₁, SO₂(alkyl), SO₂(cycloalkyl), SO₂(cycloalkylalkyl), CH₂NHR₁₀, CH₂NR₁₀R₁₁, or CH₂COOR₁₀,
wherein R₁₀ and R₁₁ are each independently H, alkyl, cycloalkyl, —C(O)-alkyl, —C(O)-cycloalkyl, —C(O)OH, —C(O)—O-alkyl, —C(O)—O-cycloalkyl, —C(O)NH₂, —C(O)NH(alkyl), —C(O)NH(cycloalkyl), —C(O)N(alkyl)₂, —CH₂NH (alkyl), —CH₂COOH, —SO₂CH₃, —OH, —O(alkyl), —NH₂, —NH(alkyl), or —N(alkyl)₂;
   or a pharmaceutically acceptable salt thereof; and
the C20-D3-visual-chromophore-producing compound is selected from the group consisting of C20-D3-retinol, C20-D3-retinaldehyde, C20-D3-retinyl esters, C20-D3-9-cis-retinol, C20-D3-9-cis-retinaldehye, C20-D3-9-cis-retinyl esters, C20-D3-11-cis-retinol, C20-D3-11-cis-retinaldehye, C20-D3-11-cis-retinyl esters, and C20-D3-C20′-D3-β-carotene.

In some embodiments, the bispecific RBP4/TTR ligand is the compound wherein m is 1 or 2 and n is 0, 1, or 2.

In some embodiments, the bispecific RBP4/TTR ligand is the compound wherein m is 1 and n is 1.

In some embodiments, the bispecific RBP4/TTR ligand is the compound wherein Y and Z are each independently CH₂, O, S, or NH.

In some embodiments, the bispecific RBP4/TTR ligand is the compound wherein Y is O and Z is CH₂.

In some embodiments, the bispecific RBP4/TTR ligand is the compound wherein A and B are N, C and D are CR₉, and E is C—F-G.

In some embodiments, the bispecific RBP4/TTR ligand is the compound wherein A, B, C, and D are CR₉, and E is C—F-G.

In some embodiments, the bispecific RBP4/TTR ligand is the compound wherein A is N, B is C—F-G, and C, D, and E are each CR₉.

In some embodiments, the bispecific RBP4/TTR ligand is the compound wherein A is N, B, C, and D are each CR₉, and E is C—F-G.

In some embodiments, the bispecific RBP4/TTR ligand is the compound wherein A, C, and D are each CR₉, B is N, and E is C—F-G.

In some embodiments, the bispecific RBP4/TTR ligand is the compound wherein:
X is CR₆ or N; and
R₁, R₂, R₃, R₄, and R₆ are each independently H, t-butyl,
cyclopentyl, cyclohexyl, CF₃, F, Cl, CN, or —OCH₃.

In some embodiments, the bispecific RBP4/TTR ligand is the compound wherein R₁ or R₄ is CF₃.

In some embodiments, the bispecific RBP4/TTR ligand is the compound wherein
X is CR₆,
R₁ is CF₃, and
R₂, R₃, R₄, and R₆ are each independently H, t-butyl,
cyclopentyl, cyclohexyl, CF₃, F, Cl, CN, or —OCH₃.

In some embodiments, the bispecific RBP4/TTR ligand is the compound having the structure:

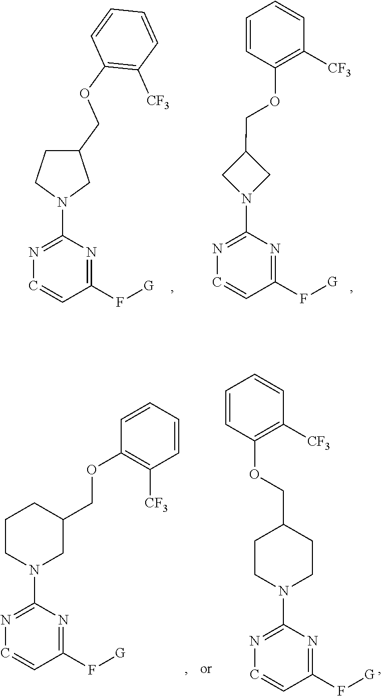

wherein
   C is CR₉;
   R₉ is H, halogen, —OH, alkyl, cycloalkyl, cycloalkylalkyl, —O-(alkyl), —S-(alkyl), —NH₂, —NH(alkyl), —NH(alkyl), —CO₂H, or —CO(O-alkyl);
   F is absent or present, and when present, is

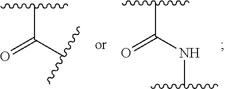

and
G is H, substituted or unsubstituted monocycle, bicycle, heteromonocycle, heterobicycle, aryl, heteroaryl, alkyl, cycloalkyl, cycloalkylalkyl, CO₂H, COOR₁₀, OH, OR₁₀, NH₂, NHR₁₀, NR₁₀R₁₁, SO₂(alkyl), SO₂(cycloalkyl), SO₂(cycloalkylalkyl), CH₂NHR₁₀, CH₂NR₁₀R₁₁, or CH₂COOR₁₀,
wherein R₁₀ and R₁₁ are each independently H, alkyl, cycloalkyl, —C(O)-alkyl, —C(O)-cycloalkyl, —C(O)OH, —C(O)—O-alkyl, —C(O)—O-cycloalkyl, —C(O)NH₂, —C(O)NH(alkyl), —C(O)NH(cycloalkyl), —C(O)N(alkyl)₂, —CH₂NH (alkyl), —CH₂COOH, —SO₂CH₃, —OH, —O(alkyl), —NH₂, —NH(alkyl), or —N(alkyl)₂;
   or a pharmaceutically acceptable salt thereof.

In some embodiments, the bispecific RBP4/TTR ligand is the compound wherein
C is CR₉ and
R₉ is H, alkyl, —O(alkyl), or —NH(alkyl).

In some embodiments, the bispecific RBP4/TTR ligand is the compound wherein R₉ is -alkyl.

In some embodiments, the bispecific RBP4/TTR ligand is the compound having the structure

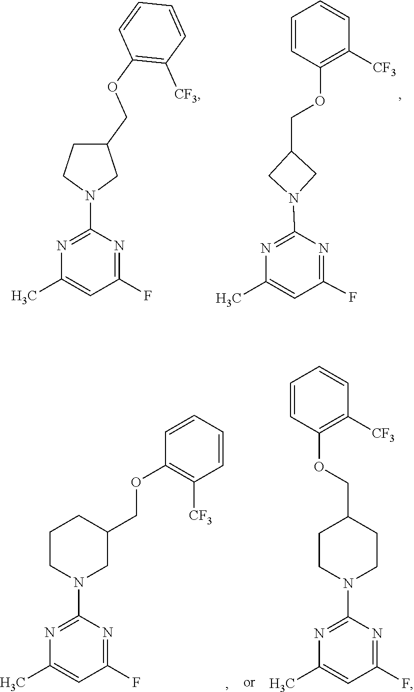

wherein F is a substituted or unsubstituted heteroaryl group.

In some embodiments, the bispecific RBP4/TTR ligand is the compound wherein F has the structure

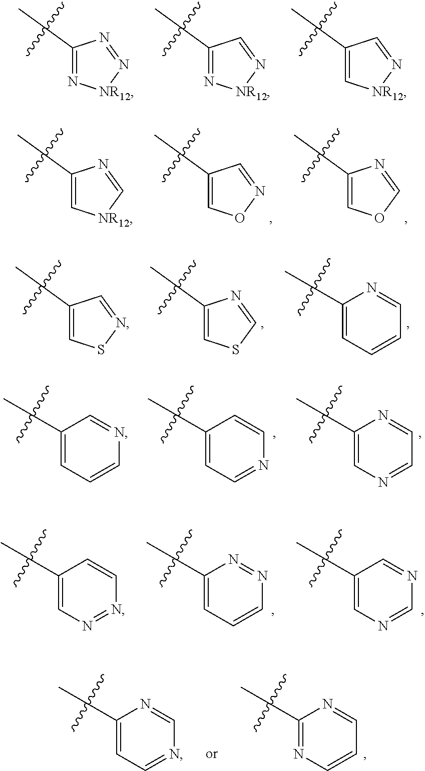

wherein R₁₂ is H, -(alkyl), -(alkenyl), or -(alkynyl).

In some embodiments, the bispecific RBP4/TTR ligand is the compound having the structure:

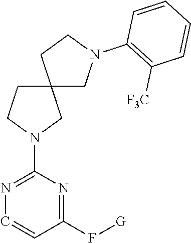

wherein
   C is CR₉;
   R₉ is H, halogen, —OH, alkyl, cycloalkyl, cycloalkylalkyl, —O-(alkyl), —S-(alkyl), —NH₂, —NH(alkyl), —NH(alkyl), —CO₂H, or —CO(O-alkyl);
   F is absent or present, and when present, is

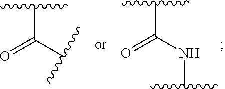

and
G is H, substituted or unsubstituted monocycle, bicycle, heteromonocycle, heterobicycle, aryl, heteroaryl, alkyl, cycloalkyl, cycloalkylalkyl, CO₂H, COOR₁₀, OH, OR₁₀, NH₂, NHR₁₀, NR₁₀R₁₁, SO₂(alkyl), SO₂(cycloalkyl), SO₂(cycloalkylalkyl), CH₂NHR₁₀, CH₂NR₁₀R₁₁, or CH₂COOR₁₀,
wherein R₁₀ and R₁₁ are each independently H, alkyl, cycloalkyl, —C(O)-alkyl, —C(O)-cycloalkyl, —C(O)OH, —C(O)—O-alkyl, —C(O)—O-cycloalkyl, —C(O)NH₂, —C(O)NH(alkyl), —C(O)NH(cycloalkyl), —C(O)N(alkyl)₂, —CH₂NH (alkyl), —CH₂COOH, —SO₂CH₃, —OH, —O(alkyl), —NH₂, —NH(alkyl), or —N(alkyl)₂;
   or a pharmaceutically acceptable salt thereof.

In some embodiments, the bispecific RBP4/TTR ligand is the compound wherein
C is CR₉ and
R₉ is H, alkyl, —O(alkyl), or —NH(alkyl).

In some embodiments, the bispecific RBP4/TTR ligand is the compound wherein R₉ is -alkyl.

In some embodiments, the bispecific RBP4/TTR ligand is the compound having the structure

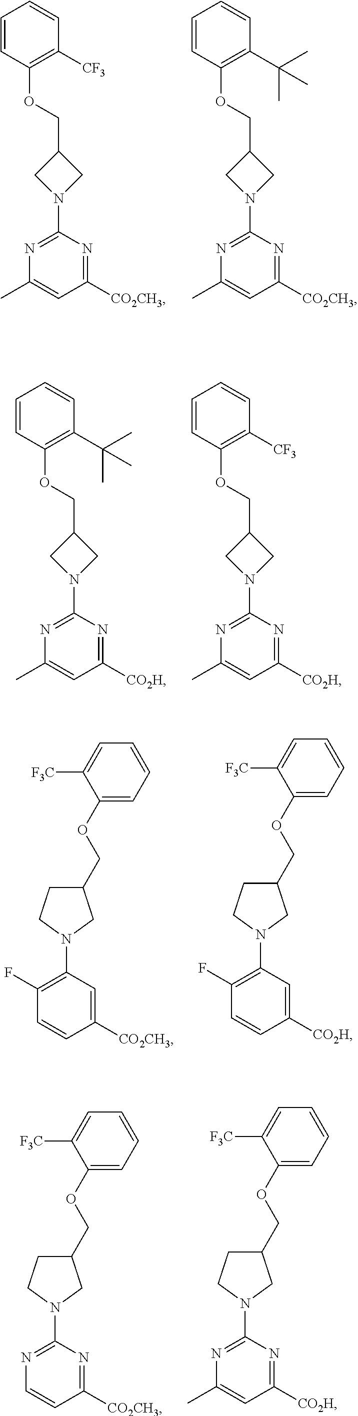

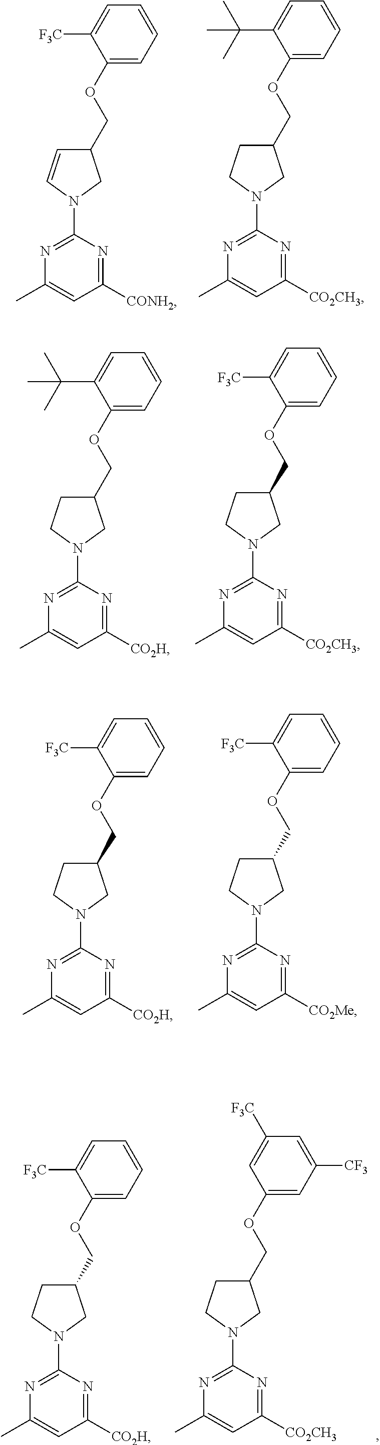

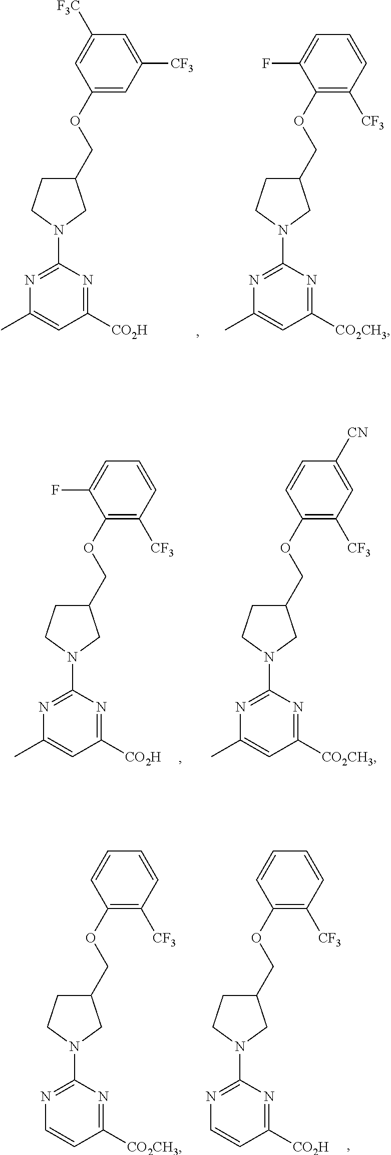 [SMILES: Cc1cc(C(=O)O)nc(N2CCC(COc3cc(C(F)(F)F)cc(C(F)(F)F)c3)C2)n1 ; COC(=O)c1cc(C)nc(N2CCC(COc3c(F)cccc3C(F)(F)F)C2)n1 ; Cc1cc(C(=O)O)nc(N2CCC(COc3c(F)cccc3C(F)(F)F)C2)n1 ; COC(=O)c1cc(C)nc(N2CCC(COc3ccc(C#N)cc3C(F)(F)F)C2)n1 ; COC(=O)c1ccnc(N2CCC(COc3ccccc3C(F)(F)F)C2)n1 ; O=C(O)c1ccnc(N2CCC(COc3ccccc3C(F)(F)F)C2)n1]

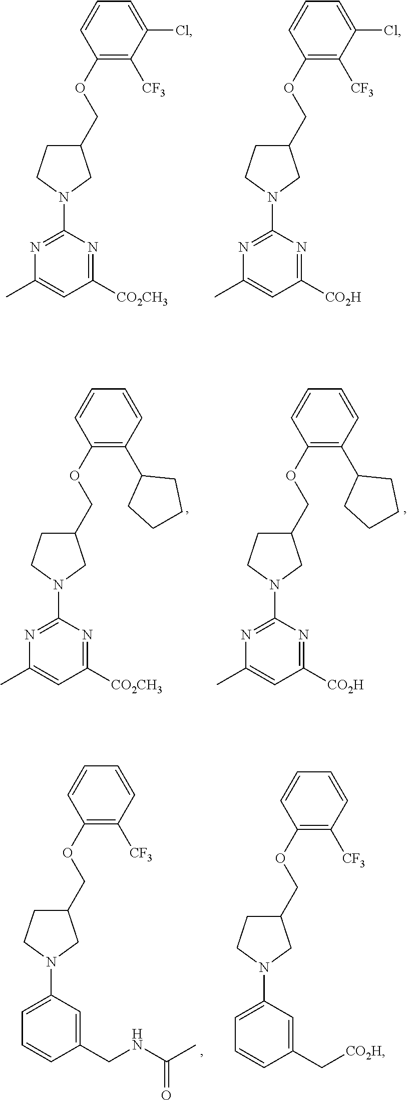

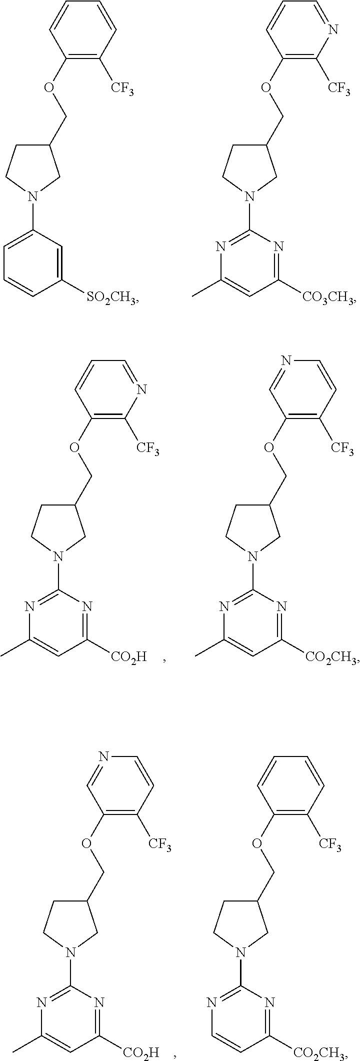

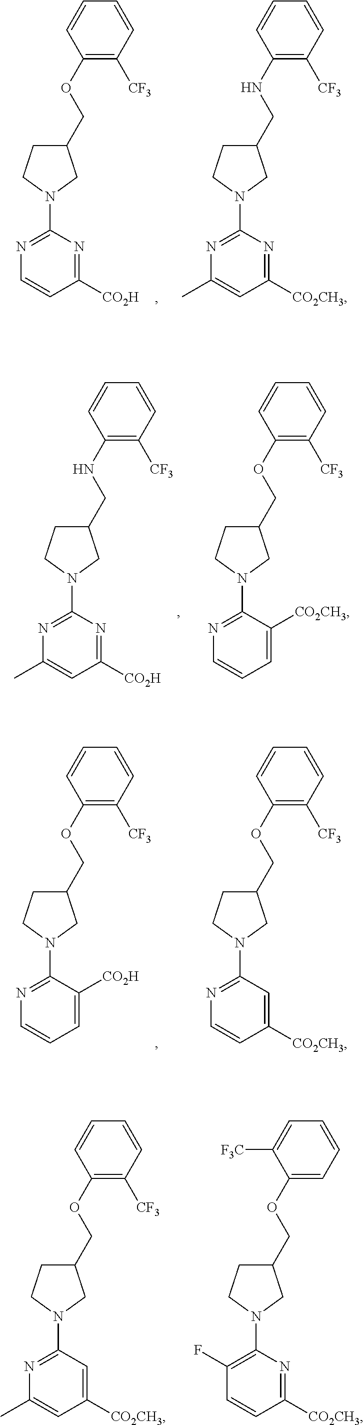

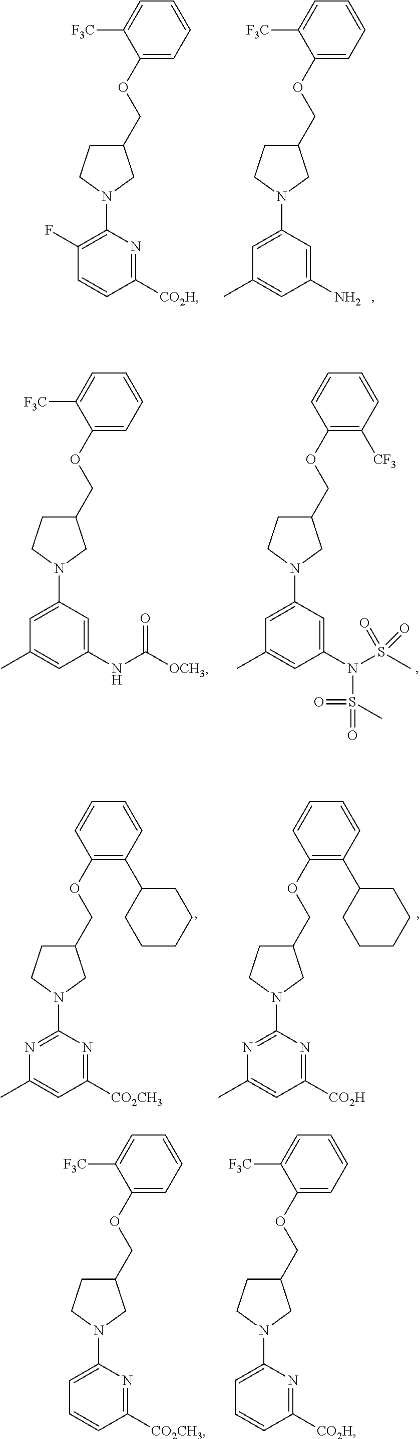

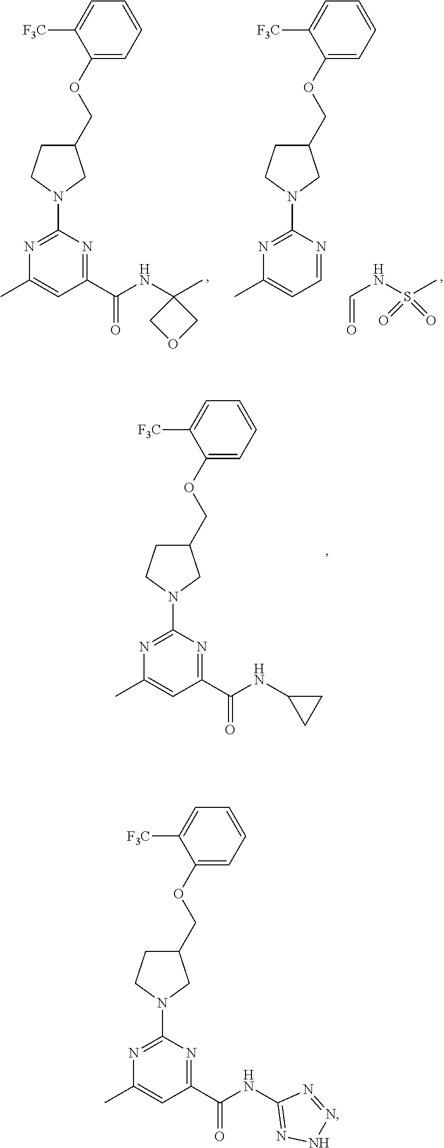

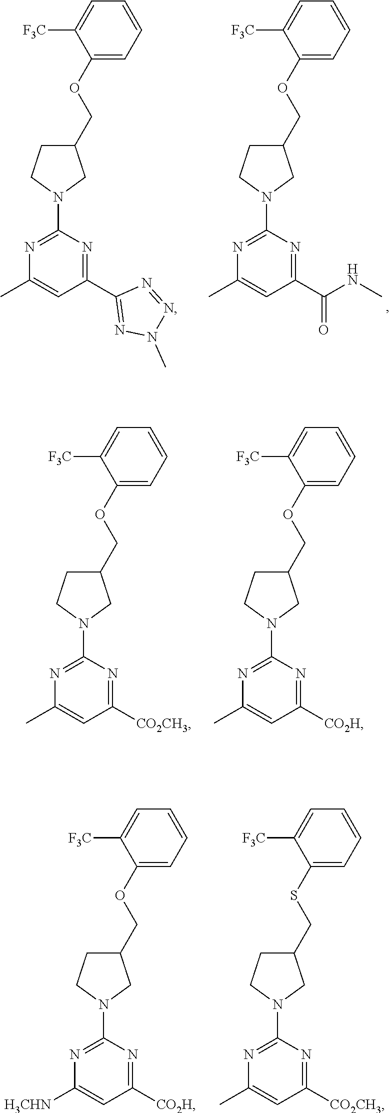

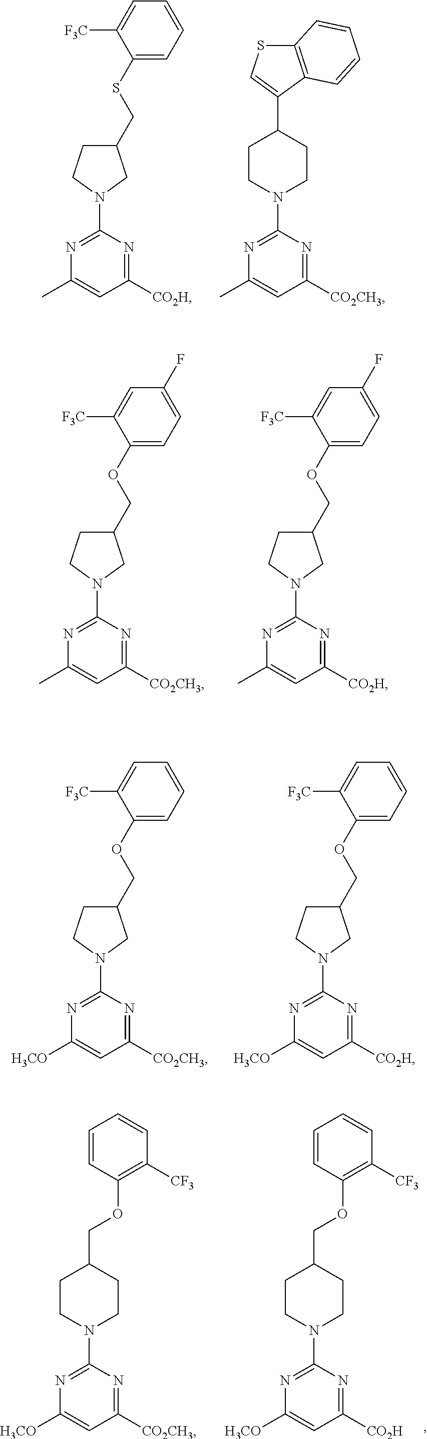

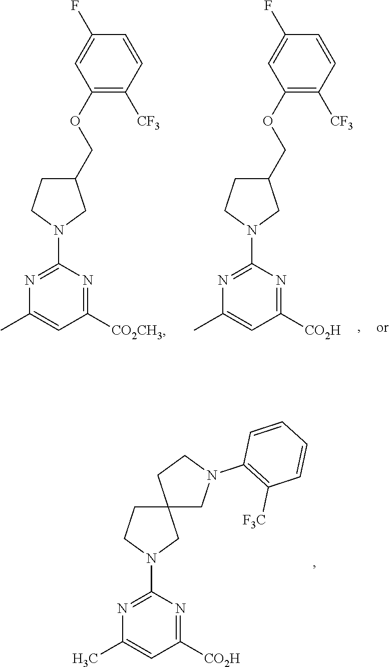 [SMILES: COC(=O)c1cc(C)nc(N2CCC(COc3cc(F)ccc3C(F)(F)F)C2)n1 ; Cc1cc(C(=O)O)nc(N2CCC(COc3cc(F)ccc3C(F)(F)F)C2)n1 ; "," ; Cc1cc(C(=O)O)nc(N2CCC3(CCN(c4ccccc4C(F)(F)F)C3)C2)n1 ; ","]

or a pharmaceutically acceptable salt of the compound.

In some embodiments, the bispecific RBP4/TTR ligand is the compound having the structure

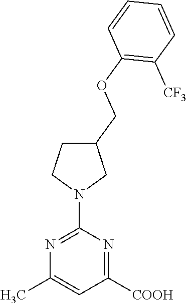 [SMILES: Cc1cc(C(=O)O)nc(N2CCC(COc3ccccc3C(F)(F)F)C2)n1]

or a pharmaceutically acceptable salt of the compound.

In some embodiments, the bispecific RBP4/TTR ligand is the compound having the structure

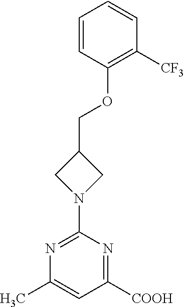 [SMILES: Cc1cc(C(=O)O)nc(N2CC(COc3ccccc3C(F)(F)F)C2)n1]

or a pharmaceutically acceptable salt of the compound.

In some embodiments, the bispecific RBP4/TTR ligand is the compound having the structure

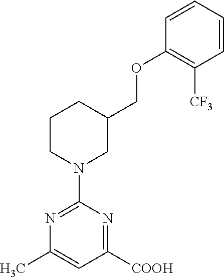 [SMILES: Cc1cc(C(=O)O)nc(N2CCCC(COc3ccccc3C(F)(F)F)C2)n1]

or a pharmaceutically acceptable salt of the compound.

In some embodiments, the bispecific RBP4/TTR ligand is the compound having the structure

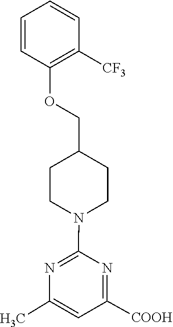 [SMILES: Cc1cc(C(=O)O)nc(N2CCC(COc3ccccc3C(F)(F)F)CC2)n1]

or a pharmaceutically acceptable salt of the compound.

In some embodiments, the bispecific RBP4/TTR ligand is the compound having the structure

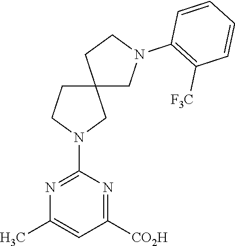 [SMILES: Cc1cc(C(=O)O)nc(N2CCC3(CCN(c4ccccc4C(F)(F)F)C3)C2)n1]

or a pharmaceutically acceptable salt of the compound.

In some embodiments, the C20-D3-visual-chromophore-producing compound is in a form packaged in chylomicrons.

The present disclosure provides a method for stabilizing TTR tetramers and for promoting rhodopsin and cone opsins production in a mammal, comprising administering to the mammal an effective amount of the pharmaceutical composition of the present disclosure to effectively stabilize TTR tetramers and to promote rhodopsin and cone opsins production in the mammal by a simultaneous, contemporaneous, or concomitant administration of the bispecific RBP4/TTR ligand and the C20-D3-visual-chromophore-producing compound.

The present disclosure provides a method for treating a disease characterized by excessive or age-related lipofuscin accumulation in the retina, or a TTR amyloidosis (ATTR) disease, or both a disease characterized by excessive or age-related lipofuscin accumulation and a TTR amyloidosis (ATTR) disease, in a mammal afflicted therewith, and for promoting rhodopsin and cone opsins production, comprising administering to the mammal an effective amount of the pharmaceutical composition of the present disclosure for a simultaneous, contemporaneous, or concomitant administration of the bispecific RBP4/TTR ligand and the C20-D3-visual-chromophore-producing compound.

In some embodiments of the method, the disease is further characterized by bisretinoid-mediated macular degeneration.

In some embodiments of the method, the amount of the bispecific RBP4/TTR ligand is effective to lower the serum concentration of RBP4 in the mammal, or the amount of the bispecific RBP4/TTR ligand is effective to lower the retinal concentration of a bisretinoid in lipofuscin in the mammal.

In some embodiments of the method, the amount of the bispecific RBP4/TTR ligand is effective to stabilize TTR tetramers in the mammal.

In some embodiments of the method, the bisretinoid is A2E.

In some embodiments of the method, the bisretinoid is isoA2E.

In some embodiments of the method, the bisretinoid is A2-DHP-PE.

In some embodiments of the method, the bisretinoid is atRAL di-PE.

In some embodiments of the method, the disease characterized by age-related lipofuscin accumulation in the retina is Age-Related Macular Degeneration.

In some embodiments of the method, the disease characterized by age-related lipofuscin accumulation in the retina is dry (atrophic) Age-Related Macular Degeneration.

In some embodiments of the method, the disease characterized by excessive lipofuscin accumulation in the retina is Stargardt Disease.

In some embodiments of the method, the disease characterized by excessive lipofuscin accumulation in the retina is other forms of retinopathy caused by or associated with mutations in the ABCA4 gene, such as retinitis pigmentosa (RP19) or cone-rod dystrophy (CORD3).

In some embodiments of the method, the disease characterized by excessive lipofuscin accumulation in the retina is Best disease.

In some embodiments of the method, the disease characterized by excessive lipofuscin accumulation in the retina is adult vitelliform maculopathy.

In some embodiments of the method, the disease characterized by excessive lipofuscin accumulation in the retina is Stargardt-like macular dystrophy.

In some embodiments of the method, the administration is effective to reduce photoreceptor degeneration.

In some embodiments of the method, the method is further effective to stabilize TTR tetramers in the mammal.

In some embodiments of the method, the mammal is further afflicted with a TTR amyloidosis (ATTR) disease and the method is effective for treating the TTR amyloidosis (ATTR) disease in the mammal.

In some embodiments of the method, the TTR amyloidosis (ATTR) disease is senile systemic amyloidosis (SSA).

In some embodiments of the method, the TTR amyloidosis (ATTR) disease is peripheral polyneuropathy (ATTR-PN).

In some embodiments of the method, the TTR amyloidosis (ATTR) disease is cardiomyopathy (ATTR-CM).

In some embodiments of the method, the TTR amyloidosis (ATTR) disease is characterized by deposition of amyloid aggregates.

In some embodiments, the bisretinoid-mediated macular degeneration is Age-Related Macular Degeneration or Stargardt Disease.

In some embodiments, the bisretinoid-mediated macular degeneration is Age-Related Macular Degeneration.

In some embodiments, the bisretinoid-mediated macular degeneration is dry (atrophic) Age-Related Macular Degeneration.

In some embodiments, the bisretinoid-mediated macular degeneration is Stargardt Disease.

In some embodiments, the bisretinoid-mediated macular degeneration is other forms of retinopathy caused by or associated with mutations in the ABCA4 gene, such as retinitis pigmentosa (RP19) or cone-rod dystrophy (CORD3).

In some embodiments, the bisretinoid-mediated macular degeneration is Best disease.

In some embodiments, the bisretinoid-mediated macular degeneration is adult vitelliform maculopathy.

In some embodiments, the bisretinoid-mediated macular degeneration is Stargardt-like macular dystrophy.

In some embodiments, TTR amyloidosis (ATTR) diseases are characterized by the deposition of amyloid aggregates.

In some embodiments, TTR amyloidosis (ATTR) diseases are characterized by the deposition of amyloid aggregates derived from either mutant (TTRm) or wild-type (TTRwt).

In some embodiments, TTR amyloidosis (ATTR) disease is senile systemic amyloidosis (SSA).

In some embodiments, TTR amyloidosis (ATTR) disease is peripheral polyneuropathy (ATTR-PN).

In some embodiments, TTR amyloidosis (ATTR) disease is cardiomyopathy (ATTR-CM).

In some embodiments, the pharmaceutical composition of the present disclosure exhibits dual retinol-binding protein 4 (RBP4) antagonist and transthyretin (TTR) tetramer kinetic stabilization activity as well as activity of promoting rhodopsin and cone opsins production.

In some embodiments, the pharmaceutical composition of the present disclosure exhibits retinol-binding protein 4 (RBP4) antagonist activity as well as activity of promoting rhodopsin and cone opsins production.

In some embodiments, the pharmaceutical composition of the present disclosure exhibits transthyretin (TTR) tetramer kinetic stabilization activity as well as activity of promoting rhodopsin and cone opsins production.

In some embodiments, the pharmaceutical composition of the present disclosure reduces circulating RBP4 levels while simultaneously stabilizing unliganded TTR tetramers released from the holo-RBP4-TTR complex, and promotes rhodopsin and cone opsins production.

In some embodiments, the pharmaceutical composition of the present disclosure reduces circulating RBP4 levels and promotes rhodopsin and cone opsins production.

In some embodiments, the pharmaceutical composition of the present disclosure stabilizes unliganded TTR tetramers released from the holo-RBP4-TTR complex and promotes rhodopsin and cone opsins production.

In some embodiments, the pharmaceutical composition of the present disclosure may be used for the treatment of dry age-related macular degeneration (AMD) and TTR amyloidosis (ATTR) comorbidities.

In some embodiments, the pharmaceutical composition of the present disclosure may be used for the treatment of dry age-related macular degeneration (AMD) and senile systemic amyloidosis (SSA).

In some embodiments, the pharmaceutical composition of the present disclosure may be used for the treatment of dry age-related macular degeneration (AMD) and peripheral polyneuropathy (ATTR-PN).

In some embodiments, the pharmaceutical composition of the present disclosure may be used for the treatment of dry age-related macular degeneration (AMD) and cardiomyopathy (ATTR-CM).

In some embodiments, the pharmaceutical composition of the present disclosure may be used for the treatment of type 2 diabetes.

In some embodiments, the pharmaceutical composition of the present disclosure may be used for the treatment of obesity.

In some embodiments, the pharmaceutical composition of the present disclosure may be used for the treatment of insulin resistance.

In some embodiments, the pharmaceutical composition of the present disclosure may be used for the treatment of cardiovascular disease.

In some embodiments, the pharmaceutical composition of the present disclosure may be used for the treatment of hepatic steatosis.

In some embodiments, the pharmaceutical composition of the present disclosure may be used for the treatment of non-alcoholic fatty liver disease (NAFLD).

In some embodiments of the method, the administration is oral.

In some embodiments, the mammal is a human.

The present disclosure provides a method for stabilizing TTR tetramers and for promoting rhodopsin and cone opsins production in a mammal, comprising the steps of sequentially, simultaneously, contemporaneously, or concomitantly administering to the mammal an amount of a bispecific RBP4/TTR ligand effective to stabilize TTR tetramers and an amount of a C20-D3-visual-chromophore-producing compound effective to promote rhodopsin and cone opsins production, in which in a sequential administration, the bispecific RBP4/TTR ligand may be administered first or the C20-D3-visual-chromophore-producing compound may be administered first.

The present disclosure provides a method for treating a disease characterized by excessive or age-related lipofuscin accumulation in the retina, or a TTR amyloidosis (ATTR) disease, or both a disease characterized by excessive or age-related lipofuscin accumulation and a TTR amyloidosis (ATTR) disease, in a mammal afflicted therewith, and for promoting rhodopsin and cone opsins production, comprising the steps of sequentially, simultaneously, contemporaneously, or concomitantly administering to the mammal an amount of a bispecific RBP4/TTR ligand effective to treat a disease characterized by excessive or age-related lipofuscin accumulation in the retina, or a TTR amyloidosis (ATTR) disease, or both a disease characterized by excessive or age-related lipofuscin accumulation and a TTR amyloidosis (ATTR) disease, and an amount of a C20-D3-visual-chromophore-producing compound effective to promote rhodopsin and cone opsins production, in which in a sequential administration, the bispecific RBP4/TTR ligand may be administered first or the C20-D3-visual-chromophore-producing compound may be administered first.

In some embodiments of the method, the disease is further characterized by bisretinoid-mediated macular degeneration.

In some embodiments of the method, the amount of the bispecific RBP4/TTR ligand is effective to lower the serum concentration of RBP4 in the mammal, or the amount of the bispecific RBP4/TTR ligand is effective to lower the retinal concentration of a bisretinoid in lipofuscin in the mammal.

In some embodiments of the method, the amount of the bispecific RBP4/TTR ligand is effective to stabilize TTR tetramers in the mammal.

In some embodiments of the method, the bisretinoid is A2E.

In some embodiments of the method, the bisretinoid is isoA2E.

In some embodiments of the method, the bisretinoid is A2-DHP-PE.

In some embodiments of the method, the bisretinoid is atRAL di-PE.

In some embodiments of the method, the disease characterized by age-related lipofuscin accumulation in the retina is Age-Related Macular Degeneration.

In some embodiments of the method, the disease characterized by age-related lipofuscin accumulation in the retina is dry (atrophic) Age-Related Macular Degeneration.

In some embodiments of the method, the disease characterized by excessive lipofuscin accumulation in the retina is Stargardt Disease.

In some embodiments of the method, the disease characterized by excessive lipofuscin accumulation in the retina is other forms of retinopathy caused by or associated with mutations in the ABCA4 gene, such as retinitis pigmentosa (RP19) or cone-rod dystrophy (CORD3).

In some embodiments of the method, the disease characterized by excessive lipofuscin accumulation in the retina is Best disease.

In some embodiments of the method, the disease characterized by excessive lipofuscin accumulation in the retina is adult vitelliform maculopathy.

In some embodiments of the method, the disease characterized by excessive lipofuscin accumulation in the retina is Stargardt-like macular dystrophy.

In some embodiments of the method, the administration is effective to reduce photoreceptor degeneration.

In some embodiments of the method, the method is further effective to stabilize TTR tetramers in the mammal.

In some embodiments of the method, the mammal is further afflicted with a TTR amyloidosis (ATTR) disease and the method is effective for treating the TTR amyloidosis (ATTR) disease in the mammal.

In some embodiments of the method, the TTR amyloidosis (ATTR) disease is senile systemic amyloidosis (SSA).

In some embodiments of the method, the TTR amyloidosis (ATTR) disease is peripheral polyneuropathy (ATTR-PN).

In some embodiments of the method, the TTR amyloidosis (ATTR) disease is cardiomyopathy (ATTR-CM).

In some embodiments of the method, the TTR amyloidosis (ATTR) disease is characterized by deposition of amyloid aggregates.

In some embodiments of the method, the bisretinoid-mediated macular degeneration is Age-Related Macular Degeneration or Stargardt Disease.

In some embodiments of the method, the bisretinoid-mediated macular degeneration is Age-Related Macular Degeneration.

In some embodiments of the method, the bisretinoid-mediated macular degeneration is dry (atrophic) Age-Related Macular Degeneration.

In some embodiments of the method, the bisretinoid-mediated macular degeneration is Stargardt Disease.

In some embodiments of the method, the bisretinoid-mediated macular degeneration is other forms of retinopathy caused by or associated with mutations in the ABCA4 gene, such as retinitis pigmentosa (RP19) or cone-rod dystrophy (CORD3).

In some embodiments of the method, the bisretinoid-mediated macular degeneration is Best disease.

In some embodiments of the method, the bisretinoid-mediated macular degeneration is adult vitelliform maculopathy.

In some embodiments of the method, the bisretinoid-mediated macular degeneration is Stargardt-like macular dystrophy.

In some embodiments of the method, TTR amyloidosis (ATTR) diseases are characterized by the deposition of amyloid aggregates.

In some embodiments of the method, TTR amyloidosis (ATTR) diseases are characterized by the deposition of amyloid aggregates derived from either mutant (TTRm) or wild-type (TTRwt).

In some embodiments of the method, TTR amyloidosis (ATTR) disease is senile systemic amyloidosis (SSA).

In some embodiments of the method, TTR amyloidosis (ATTR) disease is peripheral polyneuropathy (ATTR-PN).

In some embodiments of the method, TTR amyloidosis (ATTR) disease is cardiomyopathy (ATTR-CM).

In some embodiments of the method, the administration is oral.

In some embodiments of the method, the mammal is a human.

In some embodiments of the method, the C20-D3-visual-chromophore-producing compound is selected from the group consisting of C20-D3-retinol, C20-D3-retinaldehyde, C20-D3-retinyl esters, C20-D3-9-cis-retinol, C20-D3-9-cis-retinaldehye, C20-D3-9-cis-retinyl esters, C20-D3-11-cis-retinol, C20-D3-11-cis-retinaldehye, C20-D3-11-cis-retinyl esters, and C20-D3-C20′-D3-β-carotene.

In some embodiments of the method, the C20-D3-visual-chromophore-producing compound is in a form packaged in chylomicrons.

In some embodiments of the method, the bispecific RBP4/TTR ligand is a compound having the structure:

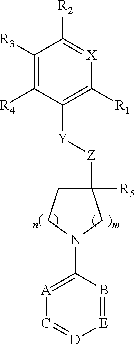

wherein
   X is CR₆ or N;
   R₁, R₂, R₃, R₄, and R₆ are each independently —H, —F, —Cl, —Br, —I, —NO₂, —CN, —CF₃, —CF₂H, —OCF₃, -(alkyl), -(haloalkyl), -(alkenyl), -(alkynyl), -(aryl), -(heteroaryl), -(cycloalkyl), -(cycloalkylalkyl), -(heteroalkyl), heterocycle, heterocycloalkyl, -(alkylheteroalkyl), -(alkylaryl), —OH, —OAc, —O-(alkyl), —O-(alkenyl), —O-(alkynyl), —O-(aryl), —O-(heteroaryl), —SH, —S-(alkyl), —S-(alkenyl), —S-(alkynyl), —S-(aryl), —S-(heteroaryl), —NH₂, —NH-(alkyl), —NH-(alkenyl), —NH-(alkynyl), —NH-(aryl), —NH-(heteroaryl), —C(O)R₇, —S(O)R₇, —SO₂R₇, —NHSO₂R₇, —OC(O)R₇, —SC(O)R₇, —NHC(O)R₈, or —NHC(S)R₈, wherein R₇ is H, -(alkyl), —OH, —O(alkyl), —NH₂, —NH(alkyl), or —N(alkyl)₂, and
wherein R₈ is -(alkyl), —O-(alkyl), —NH₂, —NH(alkyl), or —N(alkyl)₂;
   Y is O, S, N, NH, or a bond;
   Z is O, S, N, NH, (CH)ₒ, or a bond;
   R₅ is H, OH, halogen, or alkyl, or R₅ is (CH₂)ₚ and is bound to Y when Y is N to form a ring together with Z;
   o and p are independently 0, 1, 2, or 3;
   m and n are independently 0, 1, 2, 3, or 4;
   A, C, and D are each independently N or CR₉;
   R₉ is H, halogen, —OH, alkyl, cycloalkyl, cycloalkylalkyl, —O-(alkyl), —S-(alkyl), —NH₂, —NH(alkyl), —NH(alkyl)₂, —CO₂H, or —CO(O-alkyl);
   B and E are N, CR₉, or C—F-G wherein at least one of B or E is C—F-G;
   F is absent or present, and when present, is

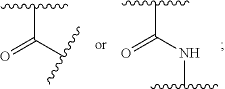

and
G is H, substituted or unsubstituted monocycle, bicycle, heteromonocycle, heterobicycle, aryl, heteroaryl, alkyl, cycloalkyl, cycloalkylalkyl, CO₂H, COOR₁₀, OH, OR₁₀, NH₂, NHR₁₀, NR₁₀R₁₁, SO₂ (alkyl), SO₂(cycloalkyl), SO₂(cycloalkylalkyl), CH₂NHR₁₀, CH₂NR₁₀R₁₁, or CH₂COOR₁₀,
wherein R₁₀ and R₁₁ are each independently H, alkyl, cycloalkyl, —C(O)-alkyl, —C(O)-cycloalkyl, —C(O)OH, —C(O)—O-alkyl, —C(O)—O-cycloalkyl, —C(O)NH₂, —C(O)NH(alkyl), —C(O)NH(cycloalkyl), —C(O)N(alkyl)₂, —CH₂NH (alkyl), —CH₂COOH, —SO₂CH₃, —OH, —O(alkyl), —NH₂, —NH(alkyl), or —N(alkyl)₂;
   or a pharmaceutically acceptable salt thereof.

In some embodiments of the method, the bispecific RBP4/TTR ligand is the compound wherein m is 1 or 2 and n is 0, 1, or 2.

In some embodiments of the method, the bispecific RBP4/TTR ligand is the compound wherein m is 1 and n is 1.

In some embodiments of the method, the bispecific RBP4/TTR ligand is the compound wherein Y and Z are each independently CH₂, O, S, or NH.

In some embodiments of the method, the bispecific RBP4/TTR ligand is the compound wherein Y is O and Z is CH₂.

In some embodiments of the method, the bispecific RBP4/TTR ligand is the compound wherein A and B are N, C and D are CR₉, and E is C—F-G.

In some embodiments of the method, the bispecific RBP4/TTR ligand is the compound wherein A, B, C, and D are CR₉, and E is C—F-G.

In some embodiments of the method, the bispecific RBP4/TTR ligand is the compound wherein A is N, B is C—F-G, and C, D, and E are each CR₉.

In some embodiments of the method, the bispecific RBP4/TTR ligand is the compound wherein A is N, B, C, and D are each CR₉, and E is C—F-G.

In some embodiments of the method, the bispecific RBP4/TTR ligand is the compound wherein A, C, and D are each CR₉, B is N, and E is C—F-G.

In some embodiments of the method, the bispecific RBP4/TTR ligand is the compound wherein:
X is CR₆ or N; and
R₁, R₂, R₃, R₄, and R₆ are each independently H, t-butyl,
cyclopentyl, cyclohexyl, CF₃, F, Cl, CN, or —OCH₃.

In some embodiments of the method, the bispecific RBP4/TTR ligand is the compound wherein R₁ or R₄ is CF₃.

In some embodiments of the method, the bispecific RBP4/TTR ligand is the compound wherein
X is CR₆,
R₁ is CF₃, and
R₂, R₃, R₄, and R₆ are each independently H, t-butyl,
cyclopentyl, cyclohexyl, CF₃, F, Cl, CN, or —OCH₃.

In some embodiments of the method, the bispecific RBP4/TTR ligand is the compound having the structure:

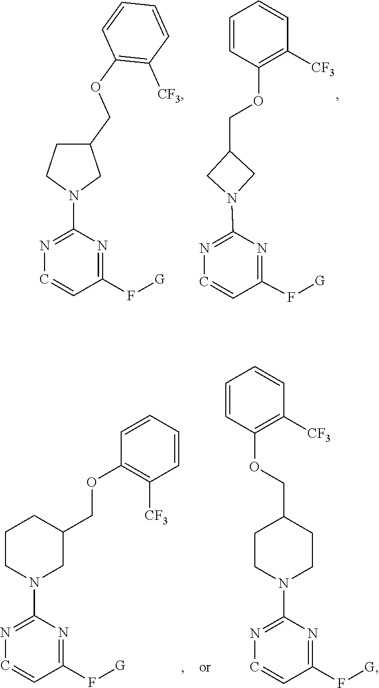

wherein
   C is CR₉;
   R₉ is H, halogen, —OH, alkyl, cycloalkyl, cycloalkylalkyl, —O-(alkyl), —S-(alkyl), —NH₂, —NH(alkyl), —NH(alkyl)₂, —CO₂H, or —CO(O-alkyl);
   F is absent or present, and when present, is

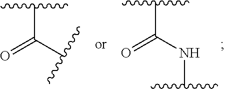

and
G is H, substituted or unsubstituted monocycle, bicycle, heteromonocycle, heterobicycle, aryl, heteroaryl, alkyl, cycloalkyl, cycloalkylalkyl, CO₂H, COOR₁₀, OH, OR₁₀, NH₂, NHR₁₀, NR₁₀R₁₁, SO₂(alkyl), SO₂(cycloalkyl), SO₂(cycloalkylalkyl), CH₂NHR₁₀, CH₂NR₁₀R₁₁, or CH₂COOR₁₀,
wherein R₁₀ and R₁₁ are each independently H, alkyl, cycloalkyl, —C(O)-alkyl, —C(O)-cycloalkyl, —C(O)OH, —C(O)—O-alkyl, —C(O)—O-cycloalkyl, —C(O)NH₂, —C(O)NH(alkyl), —C(O)NH(cycloalkyl), —C(O)N(alkyl)₂, —CH₂NH (alkyl), —CH₂COOH, —SO₂CH₃, —OH, —O(alkyl), —NH₂, —NH(alkyl), or —N(alkyl)₂;
   or a pharmaceutically acceptable salt thereof.

In some embodiments of the method, the bispecific RBP4/TTR ligand is the compound wherein
C is CR₉ and
R₉ is H, alkyl, —O(alkyl), or —NH(alkyl).

In some embodiments of the method, the bispecific RBP4/TTR ligand is the compound wherein R₉ is -alkyl.

In some embodiments of the method, the bispecific RBP4/TTR ligand is the compound having the structure

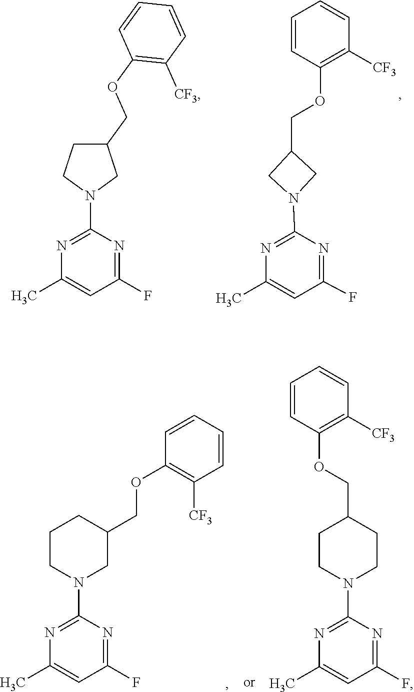

wherein F is a substituted or unsubstituted heteroaryl group.

In some embodiments of the method, the bispecific RBP4/TTR ligand is the compound wherein F has the structure

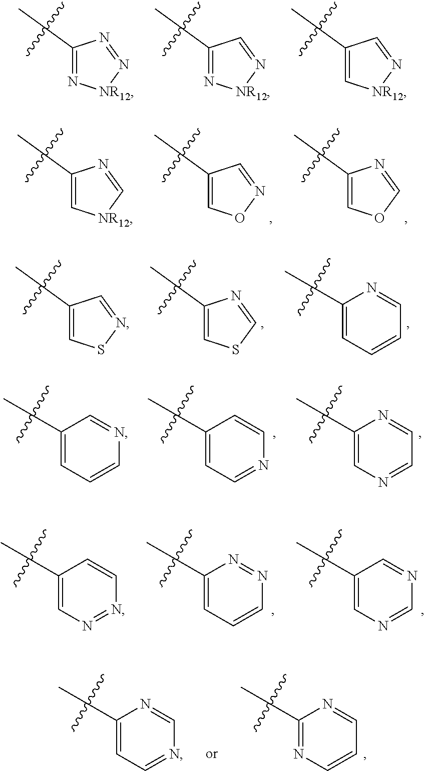

wherein R₁₂ is H, -(alkyl), -(alkenyl), or -(alkynyl).

In some embodiments of the method, the bispecific RBP4/TTR ligand is the compound having the structure:

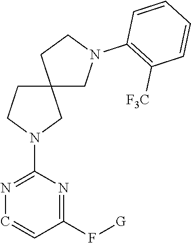

wherein
   C is CR₉;
   R₉ is H, halogen, —OH, alkyl, cycloalkyl, cycloalkylalkyl, —O-(alkyl), —S-(alkyl), —NH₂, —NH(alkyl), —NH(alkyl)₂, —CO₂H, or —CO(O-alkyl);
   F is absent or present, and when present, is

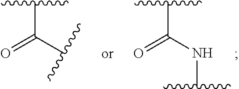

and
G is H, substituted or unsubstituted monocycle, bicycle, heteromonocycle, heterobicycle, aryl, heteroaryl, alkyl, cycloalkyl, cycloalkylalkyl, CO₂H, COOR₁₀, OH, OR₁₀, NH₂, NHR₁₀, NR₁₀R₁₁, SO₂ (alkyl), SO₂(cycloalkyl), SO₂(cycloalkylalkyl), CH₂NHR₁₀, CH₂NR₁₀R₁₁, or CH₂COOR₁₀,
wherein R₁₀ and R₁₁ are each independently H, alkyl, cycloalkyl, —C(O)-alkyl, —C(O)-cycloalkyl, —C(O)OH, —C(O)—O-alkyl, —C(O)—O-cycloalkyl, —C(O)NH₂, —C(O)NH(alkyl), —C(O)NH(cycloalkyl), —C(O)N(alkyl)₂, —CH₂NH (alkyl), —CH₂COOH, —SO₂CH₃, —OH, —O(alkyl), —NH₂, —NH(alkyl), or —N(alkyl)₂;
   or a pharmaceutically acceptable salt thereof.

In some embodiments of the method, the bispecific RBP4/TTR ligand is the compound wherein
C is CR₉ and
R₉ is H, alkyl, —O(alkyl), or —NH(alkyl).

In some embodiments of the method, the bispecific RBP4/TTR ligand is the compound wherein R₉ is -alkyl.

In some embodiments of the method, the bispecific RBP4/TTR ligand is the compound having the structure

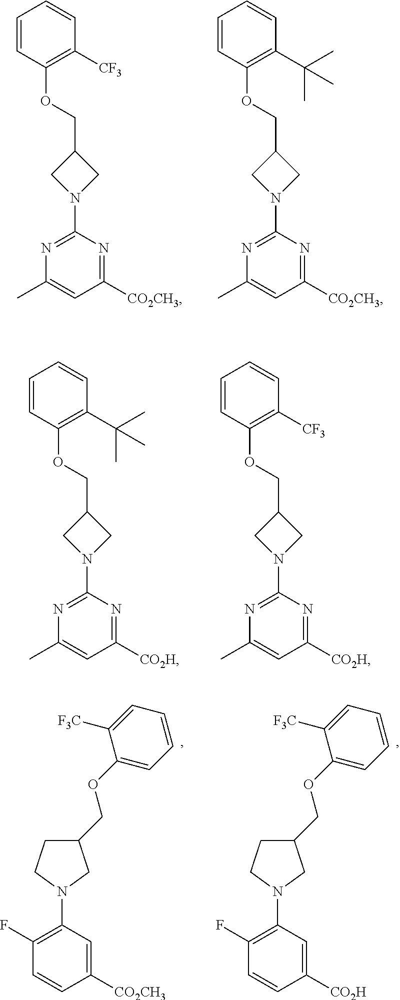 [SMILES: COC(=O)c1cc(C)nc(N2CC(COc3ccccc3C(F)(F)F)C2)n1 ; COC(=O)c1cc(C)nc(N2CC(COc3ccccc3C(C)(C)C)C2)n1 ; Cc1cc(C(=O)O)nc(N2CC(COc3ccccc3C(C)(C)C)C2)n1 ; Cc1cc(C(=O)O)nc(N2CC(COc3ccccc3C(F)(F)F)C2)n1 ; COC(=O)c1ccc(F)c(N2CCC(COc3ccccc3C(F)(F)F)C2)c1 ; O=C(O)c1ccc(F)c(N2CCC(COc3ccccc3C(F)(F)F)C2)c1]

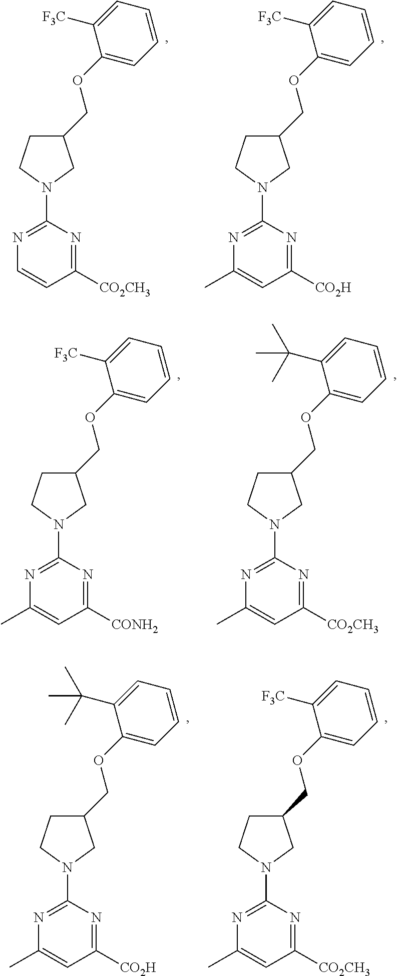

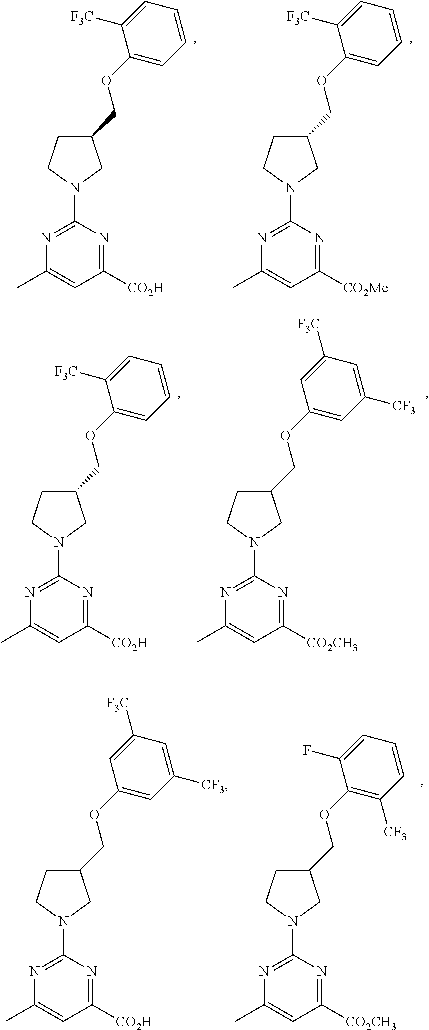

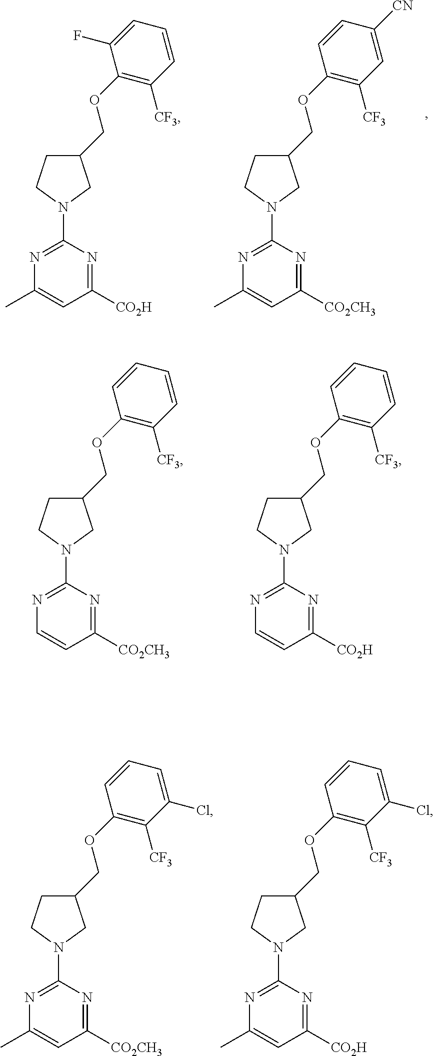

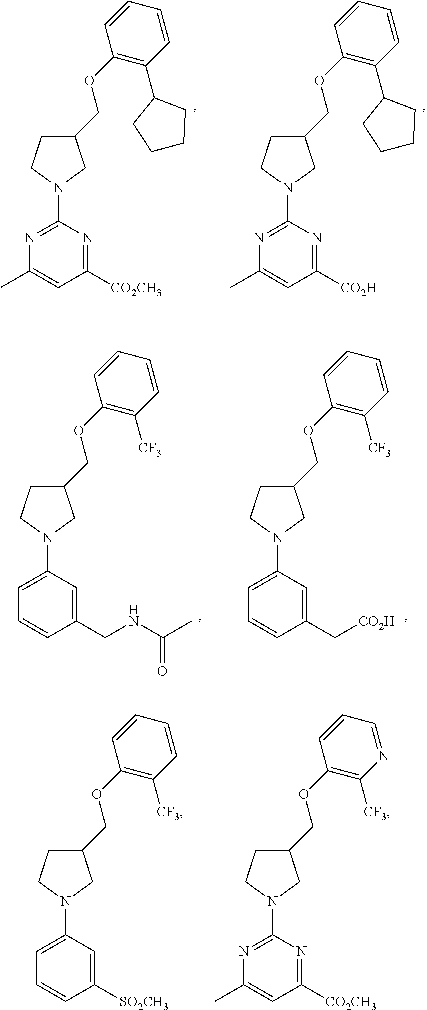 [SMILES: COC(=O)c1cc(C)nc(N2CCC(COc3ccccc3C3CCCC3)C2)n1 ; Cc1cc(C(=O)O)nc(N2CCC(COc3ccccc3C3CCCC3)C2)n1 ; CC(=O)NCc1cccc(N2CCC(COc3ccccc3C(F)(F)F)C2)c1 ; O=C(O)Cc1cccc(N2CCC(COc3ccccc3C(F)(F)F)C2)c1 ; CS(=O)(=O)c1cccc(N2CCC(COc3ccccc3C(F)(F)F)C2)c1 ; COC(=O)c1cc(C)nc(N2CCC(COc3cccnc3C(F)(F)F)C2)n1]

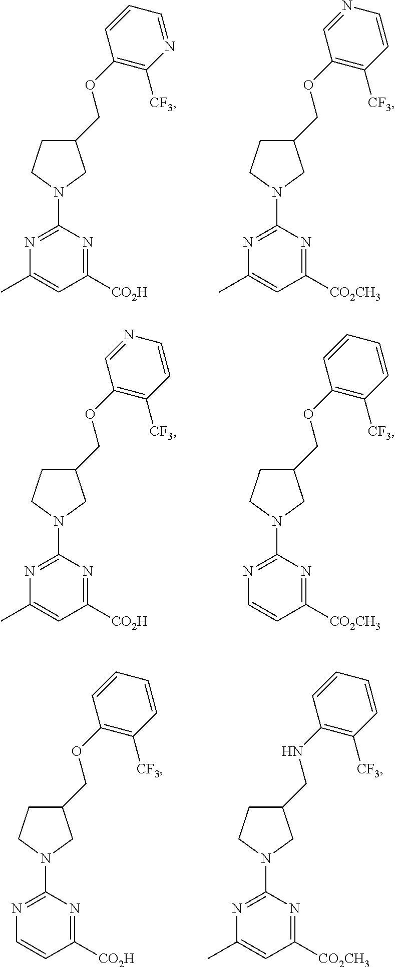

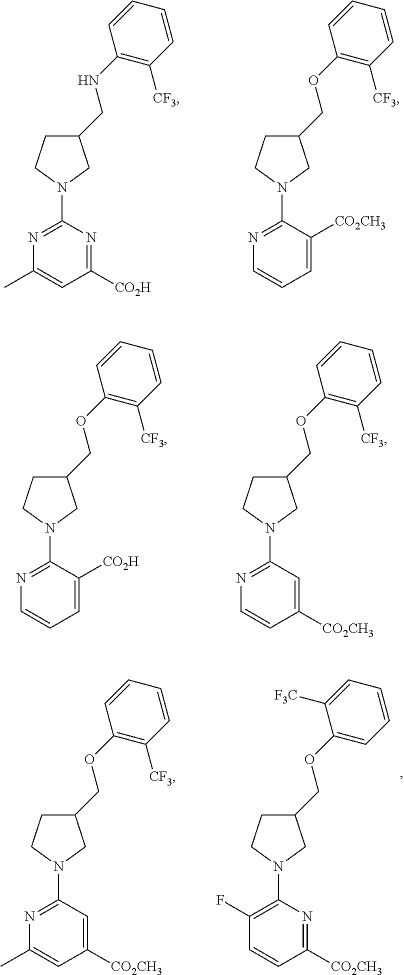

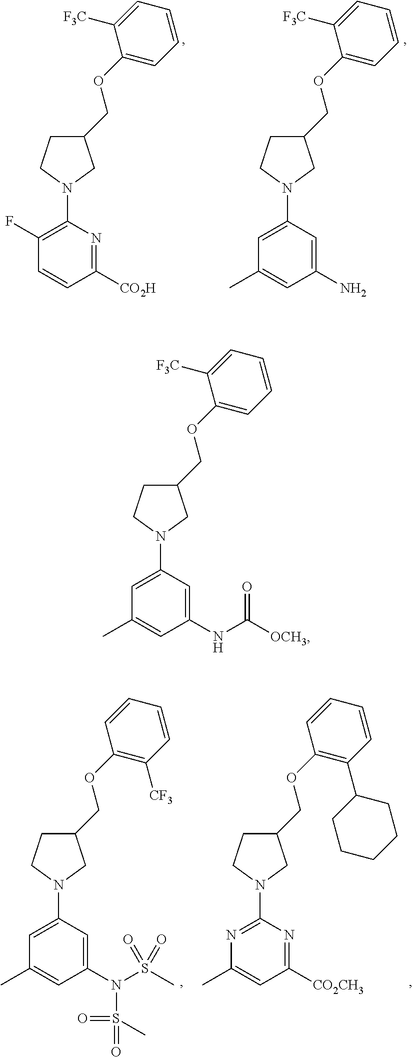

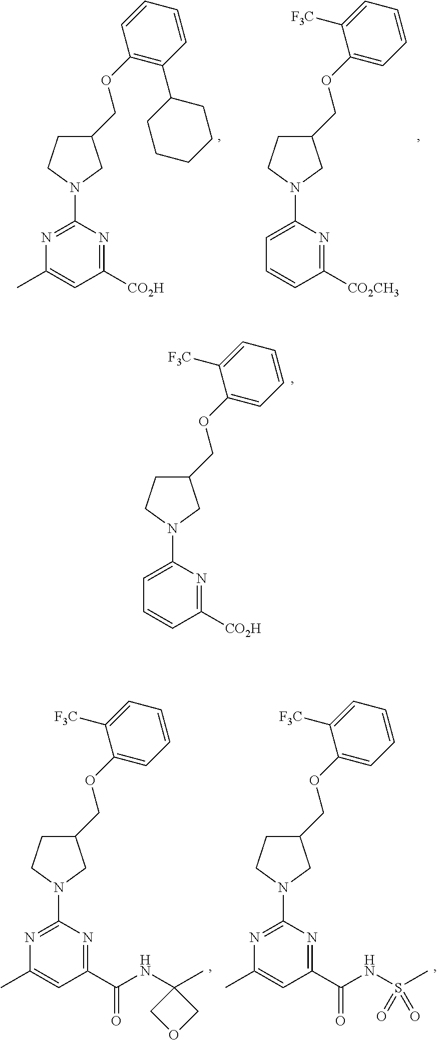

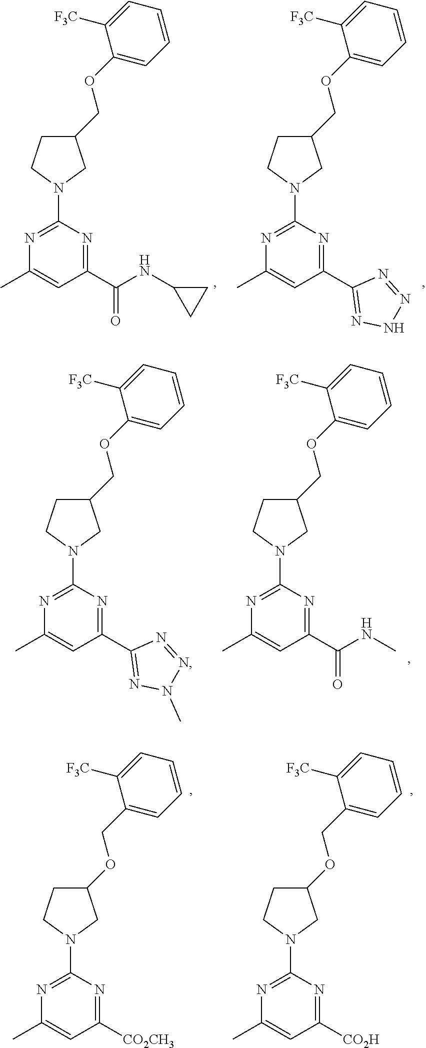

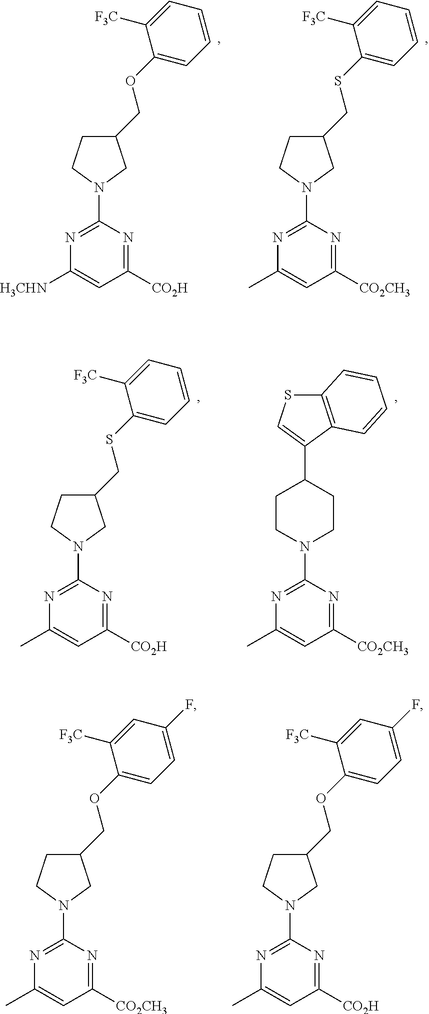

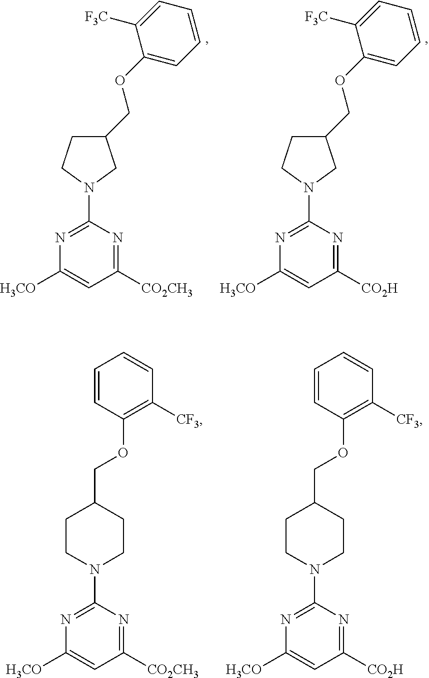 [SMILES: COC(=O)c1cc(OC)nc(N2CCC(COc3ccccc3C(F)(F)F)C2)n1 ; COc1cc(C(=O)O)nc(N2CCC(COc3ccccc3C(F)(F)F)C2)n1 ; COC(=O)c1cc(OC)nc(N2CCC(COc3ccccc3C(F)(F)F)CC2)n1 ; COc1cc(C(=O)O)nc(N2CCC(COc3ccccc3C(F)(F)F)CC2)n1]

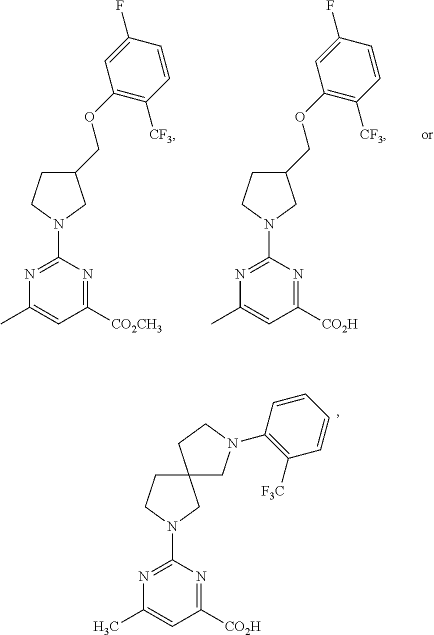 [SMILES: COC(=O)c1cc(C)nc(N2CCC(COc3cc(F)ccc3C(F)(F)F)C2)n1 ; Cc1cc(C(=O)O)nc(N2CCC(COc3cc(F)ccc3C(F)(F)F)C2)n1 ; Cc1cc(C(=O)O)nc(N2CCC3(CCN(c4ccccc4C(F)(F)F)C3)C2)n1]

or a pharmaceutically acceptable salt of the compound.

In some embodiments of the method, the bispecific RBP4/TTR ligand is the compound having the structure

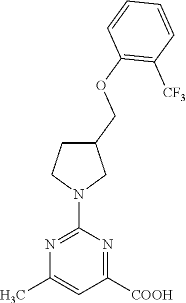 [SMILES: Cc1cc(C(=O)O)nc(N2CCC(COc3ccccc3C(F)(F)F)C2)n1]

or a pharmaceutically acceptable salt of the compound.

In some embodiments of the method, the bispecific RBP4/TTR ligand is the compound having the structure

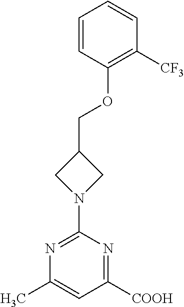 [SMILES: Cc1cc(C(=O)O)nc(N2CC(COc3ccccc3C(F)(F)F)C2)n1]

or a pharmaceutically acceptable salt of the compound.

In some embodiments of the method, the bispecific RBP4/TTR ligand is the compound having the structure

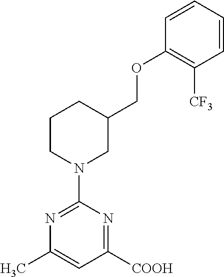 [SMILES: Cc1cc(C(=O)O)nc(N2CCCC(COc3ccccc3C(F)(F)F)C2)n1]

or a pharmaceutically acceptable salt of the compound.

In some embodiments of the method, the bispecific RBP4/TTR ligand is the compound having the structure

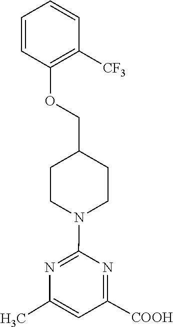 [SMILES: Cc1cc(C(=O)O)nc(N2CCC(COc3ccccc3C(F)(F)F)CC2)n1]

or a pharmaceutically acceptable salt of the compound.

In some embodiments of the method, the bispecific RBP4/TTR ligand is the compound having the structure

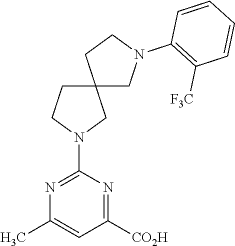 [SMILES: Cc1cc(C(=O)O)nc(N2CCC3(CCN(c4ccccc4C(F)(F)F)C3)C2)n1]

or a pharmaceutically acceptable salt of the compound.

Except where otherwise specified, if the structure of a compound of the present disclosure includes an asymmetric carbon atom, it is understood that the compound occurs as a racemate, a racemic mixture, scalemic mixtures, and isolated single enantiomers. All such isomeric forms of these compounds are expressly included in the present disclosure. Except where otherwise specified, each stereogenic carbon may be of the R or S configuration. It is to be understood accordingly that the isomers arising from such asymmetry (e.g., all enantiomers and diastereomers) are included within the scope of the present disclosure, unless indicated otherwise. Such isomers can be obtained in substantially pure form by classical separation techniques and by stereochemically controlled syntheses, such as those described in: “Enantiomers, Racemates and Resolutions” by J. Jacques, A. Collet and S. Wilen, Pub. John Wiley & Sons, NY, 1981. For example, the resolution may be carried out by preparative chromatography on a chiral column.

Except where otherwise specified, the present disclosure is intended to include all isotopes of atoms occurring on the compounds disclosed herein. Isotopes include those atoms having the same atomic number but different mass numbers. By way of a general example and without limitation, isotopes of hydrogen include tritium and deuterium. Isotopes of carbon include C-13 and C-14.

It will be noted that any notations of a carbon in structures throughout the present disclosure, when used without further notation, are intended to represent all isotopes of carbon, such as ¹²C, ¹³C, or ¹⁴C. Furthermore, any compounds containing ¹³C or ¹⁴C may specifically have the structure of any of the compounds disclosed herein.

It will also be noted that any notations of a hydrogen (H) in structures throughout the present disclosure, when used without further notation, are intended to represent all isotopes of hydrogen, such as ¹H, ²H (D), or ³H (T) except where otherwise specified. Furthermore, any compounds containing ²H or ³H may specifically have the structure of any of the compounds disclosed herein except where otherwise specified.

Isotopically labeled compounds can generally be prepared by conventional techniques known to those skilled in the art using appropriate isotopically-labeled reagents in place of the non-labeled reagents employed.

Deuterium (²H or D) is a stable, non-radioactive isotope of hydrogen and has an atomic weight of 2.0144. Hydrogen atom in a compound naturally occurs as a mixture of the isotopes ¹H (hydrogen or protium), D (2H or deuterium), and T (³H or tritium). The natural abundance of deuterium is 0.0156%. Thus, in a composition comprising molecules of a naturally occurring compound, the level of deuterium at a particular hydrogen atom site in that compound is expected to be 0.0156%. Thus, a composition comprising a compound with a level of deuterium at any site of hydrogen atom in the compound that has been enriched to be greater than its natural abundance of 0.0156% is novel over its naturally occurring counterpart. A person skilled in the art may use the techniques disclosed herein to prepare deuterium analogs thereof.

The term “substitution”, “substituted” and “substituent” refers to a functional group as described above in which one or more bonds to a hydrogen atom contained therein are replaced by a bond to non-hydrogen or non-carbon atoms, provided that normal valencies are maintained and that the substitution results in a stable compound. Substituted groups also include groups in which one or more bonds to a carbon or hydrogen atom(s) are replaced by one or more bonds, including double or triple bonds, to a heteroatom. Examples of substituent groups include: the functional groups described above; halogens (i.e., F, Cl, Br, and I); alkyl groups, such as methyl, ethyl, n-propyl, isopropryl, n-butyl, tert-butyl, and trifluoromethyl; hydroxyl; alkoxy groups, such as methoxy, ethoxy, n-propoxy, and isopropoxy; aryloxy groups, such as phenoxy; arylalkyloxy groups, such as benzyloxy (phenylmethoxy) and p-trifluoromethylbenzyloxy (4-trifluoromethylphenylmethoxy); heteroaryloxy groups; sulfonyl groups, such as trifluoromethanesulfonyl, methanesulfonyl, and p-toluenesulfonyl; nitro; nitrosyl; mercapto; sulfanyl groups, such as methylsulfanyl, ethylsulfanyl and propylsulfanyl; cyano; amino groups, such as amino, methylamino, dimethylamino, ethylamino, and diethylamino; and carboxyl. Where multiple substituent moieties are disclosed or claimed, the substituted compound can be independently substituted by one or more of the disclosed or claimed substituent moieties, singly or plurally. By independently substituted, it is meant that the (two or more) substituents can be the same or different.

In the compounds used in the method of the present disclosure, the substituents may be substituted or unsubstituted, unless specifically defined otherwise.

In the compounds used in the method of the present disclosure, alkyl, haloalkyl, alkenyl, alkynyl, aryl, heteroaryl, cycloalkyl, cycloalkylalkyl, heteroalkyl, heterocycle, heterocycloalkyl, alkylheteroalkyl, alkylaryl, monocycle, bicycle, heteromonocycle, and heterobicycle groups can be further substituted by replacing one or more hydrogen atoms with alternative non-hydrogen groups. These include, but are not limited to, halo, hydroxy, mercapto, amino, carboxy, cyano, and carbamoyl.

It is understood that substituents and substitution patterns on the compounds used in the method of the present disclosure can be selected by one of ordinary skill in the art to provide compounds that are chemically stable and that can be readily synthesized by techniques known in the art from readily available starting materials. If a substituent is itself substituted with more than one group, it is understood that these multiple groups may be on the same carbon or on different carbons, so long as a stable structure results.

In choosing the compounds used in the method of the present disclosure, one of ordinary skill in the art will recognize that the various substituents, i.e., R₁, R₂, etc., are to be chosen in conformity with well-known principles of chemical structure connectivity.

As used herein, “alkyl” includes both branched and straight-chain saturated aliphatic hydrocarbon groups having the specified number of carbon atoms and may be unsubstituted or substituted. Thus, C₁-Cₙ as in “C₁-Cₙ alkyl” is defined to include groups having 1, 2, . . . , n−1 or n carbons in a linear or branched arrangement. For example, C₁-C₆ as in “C₁-C₆ alkyl” is defined to include groups having 1, 2, 3, 4, 5, or 6 carbons in a linear or branched arrangement, and specifically includes methyl, ethyl, n-propyl, isopropyl, n-butyl, t-butyl, pentyl, and hexyl. Unless otherwise specified, an alkyl group contains one to ten carbons. Alkyl groups can be unsubstituted or substituted with one or more substituents, including but not limited to halogen, alkoxy, alkylthio, trifluoromethyl, difluoromethyl, methoxy, and hydroxyl. “Haloalkyl” includes any alkyl group containing at least one halogen atom.

The term “alkenyl” refers to a non-aromatic hydrocarbon radical, straight or branched, containing at least 1 carbon-to-carbon double bond, and up to the maximum possible number of non-aromatic carbon-carbon double bonds may be present. Thus, C₂-Cₙ alkenyl is defined to include groups having 2, 3 . . . , n−1 or n carbons. For example, “C₂-C₆ alkenyl” means an alkenyl radical having 2, 3, 4, 5, or 6 carbon atoms, and at least 1 carbon-carbon double bond, and up to, for example, 3 carbon-carbon double bonds in the case of a C₆ alkenyl, respectively. Alkenyl groups include ethenyl, propenyl, butenyl, and cyclohexenyl. As described above with respect to alkyl, the straight, branched, or cyclic portion of the alkenyl group may contain double bonds and may be substituted if a substituted alkenyl group is indicated. An embodiment can be C₂-C₁₂ alkenyl or C₂-C₈ alkenyl.

The term “alkynyl” refers to a hydrocarbon radical, straight or branched, containing at least 1 carbon-to-carbon triple bond, and up to the maximum possible number of non-aromatic carbon-carbon triple bonds may be present. Thus, C₂-Cₙ alkynyl is defined to include groups having 2, 3 . . . , n−1 or n carbons. For example, “C₂-C₆ alkynyl” means an alkynyl radical having 2 or 3 carbon atoms, and 1 carbon-carbon triple bond, or having 4 or 5 carbon atoms, and up to 2 carbon-carbon triple bonds, or having 6 carbon atoms, and up to 3 carbon-carbon triple bonds. Alkynyl groups include ethynyl, propynyl, and butynyl. As described above with respect to alkyl, the straight or branched portion of the alkynyl group may contain triple bonds and may be substituted if a substituted alkynyl group is indicated. An embodiment can be a C₂-Cₙ alkynyl. An embodiment can be C₂-C₁₂ alkynyl or C₃-C₈ alkynyl.

As used herein, “aryl” is intended to mean any stable monocyclic, bicyclic, or polycyclic carbon ring of up to 10 atoms in each ring, wherein at least one ring is aromatic, and may be unsubstituted or substituted. Examples of such aryl elements include but are not limited to: phenyl, p-toluenyl (4-methylphenyl), naphthyl, tetrahydro-naphthyl, indanyl, phenanthryl, anthryl, or acenaphthyl. In cases where the aryl substituent is bicyclic and one ring is non-aromatic, it is understood that attachment is via the aromatic ring.

The term “heteroaryl”, as used herein, represents a stable monocyclic, bicyclic, or polycyclic ring of up to 10 atoms in each ring, wherein at least one ring is aromatic and contains from 1 to 4 heteroatoms selected from the group consisting of O, N, and S. Bicyclic aromatic heteroaryl groups include phenyl, pyridine, pyrimidine, or pyridazine rings that are: (a) fused to a 6-membered aromatic (unsaturated) heterocyclic ring having one nitrogen atom; (b) fused to a 5- or 6-membered aromatic (unsaturated) heterocyclic ring having two nitrogen atoms; (c) fused to a 5-membered aromatic (unsaturated) heterocyclic ring having one nitrogen atom together with either one oxygen or one sulfur atom; or (d) fused to a 5-membered aromatic (unsaturated) heterocyclic ring having one heteroatom selected from O, N, or S. Heteroaryl groups within the scope of this definition include but are not limited to: benzimidazolyl, benzofuranyl, benzofurazanyl, benzopyrazolyl, benzotriazolyl, benzothiophenyl, benzoxazolyl, carbazolyl, carbolinyl, cinnolinyl, furanyl, indolinyl, indolyl, indolazinyl, indazolyl, isobenzofuranyl, isoindolyl, isoquinolyl, isothiazolyl, isoxazolyl, naphthpyridinyl, oxadiazolyl, oxazolyl, oxazoline, isoxazoline, oxetanyl, pyranyl, pyrazinyl, pyrazolyl, pyridazinyl, pyridopyridinyl, pyridazinyl, pyridyl, pyrimidyl, pyrrolyl, quinazolinyl, quinolyl, quinoxalinyl, tetrazolyl, tetrazolopyridyl, thiadiazolyl, thiazolyl, thienyl, triazolyl, azetidinyl, aziridinyl, 1,4-dioxanyl, hexahydroazepinyl, dihydrobenzoimidazolyl, dihydrobenzofuranyl, dihydrobenzothiophenyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, methylenedioxybenzoyl, tetrahydrofuranyl, tetrahydrothienyl, acridinyl, carbazolyl, cinnolinyl, quinoxalinyl, pyrazolyl, indolyl, benzotriazolyl, benzothiazolyl, benzoxazolyl, isoxazolyl, isothiazolyl, furanyl, thienyl, benzothienyl, benzofuranyl, quinolinyl, isoquinolinyl, oxazolyl, isoxazolyl, indolyl, pyrazinyl, pyridazinyl, pyridinyl, pyrimidinyl, pyrrolyl, and tetra-hydroquinoline. In cases where the heteroaryl substituent is bicyclic and one ring is non-aromatic or contains no heteroatoms, it is understood that attachment is via the aromatic ring or via the heteroatom-containing ring, respectively. If the heteroaryl contains nitrogen atoms, it is understood that the corresponding N-oxides thereof are also encompassed by this definition.

As used herein, “cycloalkyl” includes cyclic rings of alkanes of three to eight total carbon atoms, or any number within this range (i.e., cyclopropyl, cyclobutyl, cyclopentyl, cyclohexyl, cycloheptyl, or cyclooctyl). “Cycloalkylalkyl” includes any alkyl group containing at least one cycloalkyl ring.

As used herein, “heteroalkyl” includes both branched and straight-chain saturated aliphatic hydrocarbon groups having at least 1 heteroatom within the chain or branch. “Alkylheteroalkyl” includes any alkyl group containing at least one heteroalkyl group.

The term “heterocycle”, “heterocyclyl”, or “heterocyclic” refers to a mono- or poly-cyclic ring system which can be saturated or contains one or more degrees of unsaturation, and contains one or more heteroatoms. Preferred heteroatoms include N, O, and/or S, including N-oxides, sulfur oxides, and dioxides. Preferably the ring is three to ten-membered, and is either saturated or has one or more degrees of unsaturation. The heterocycle may be unsubstituted or substituted, with multiple degrees of substitution being allowed. Such rings may be optionally fused to one or more of another heterocyclic ring(s), heteroaryl ring(s), aryl ring(s), or cycloalkyl ring(s). Examples of heterocycles include, but are not limited to, tetrahydrofuran, pyran, 1,4-dioxane, 1,3-dioxane, piperidine, piperazine, pyrrolidine, morpholine, thiomorpholine, tetrahydrothiopyran, tetrahydrothiophene, 1,3-oxathiolane, and the like.

As used herein, “heterocyclyl” is intended to mean a 5- to 10-membered nonaromatic ring containing from 1 to 4 heteroatoms selected from the group consisting of O, N, and S, and includes bicyclic groups. “Heterocyclyl” therefore includes but is not limited to the following: imidazolyl, piperazinyl, piperidinyl, pyrrolidinyl, morpholinyl, thiomorpholinyl, tetrahydropyranyl, dihydropiperidinyl, tetrahydrothiophenyl, and the like. If the heterocycle contains nitrogen, it is understood that the corresponding N-oxides thereof are also encompassed by this definition.

The term “alkylaryl” refers to alkyl groups as described above wherein one or more bonds to hydrogen contained therein are replaced by a bond to an aryl group as described above. It is understood that an “alkylaryl” group is connected to a core molecule through a bond from the alkyl group and that the aryl group acts as a substituent on the alkyl group. Examples of alkylaryl moieties include, but are not limited to, benzyl (phenylmethyl), p-trifluoromethylbenzyl (4-trifluoromethylphenylmethyl), 1-phenylethyl, 2-phenylethyl, 3-phenylpropyl, 2-phenylpropyl, and the like.

As used herein, “monocycle” includes any stable cyclic carbon ring of up to 10 atoms and may be unsubstituted or substituted. Examples of such non-aromatic monocycle elements include but are not limited to: cyclobutyl, cyclopentyl, cyclohexyl, and cycloheptyl. Examples of such aromatic monocycle elements include but are not limited to: phenyl. As used herein, “heteromonocycle” includes any monocycle containing at least one heteroatom.

As used herein, “bicycle” includes any stable cyclic carbon ring of up to 10 atoms that is fused to a cyclic carbon ring of up to 10 atoms with each ring being independently unsubstituted or substituted. Examples of such non-aromatic bicycle elements include but are not limited to: decahydronaphthalene. Examples of such aromatic bicycle elements include but are not limited to: naphthalene. As used herein, “heterobicycle” includes any bicycle containing at least one heteroatom.

The compounds used in the method of the present disclosure may be prepared by techniques well known in organic syntheses and familiar to a practitioner ordinarily skilled in the art. However, these may not be the only means by which to synthesize or obtain the desired compounds.

The compounds used in the method of the present disclosure may be prepared by techniques described in Vogel's Textbook of Practical Organic Chemistry, A. I. Vogel, A. R. Tatchell, B. S. Furnis, A. J. Hannaford, P. W. G. Smith, (Prentice Hall) 5^{th} Edition (1996), March's Advanced Organic Chemistry: Reactions, Mechanisms, and Structure, Michael B. Smith, Jerry March, (Wiley-Interscience) 5^{th} Edition (2007), and references therein, which are incorporated by reference herein. However, these may not be the only means by which to synthesize or obtain the desired compounds.

The various R groups attached to the aromatic rings of the compounds disclosed herein may be added to the rings by standard procedures, for example, those set forth in Advanced Organic Chemistry: Part B: Reactions and Synthesis, Francis Carey and Richard Sundberg, (Springer) 5th ed. Edition. (2007), the content of which is hereby incorporated by reference.

Another aspect of the present disclosure comprises a compound or composition of the present disclosure as a pharmaceutical composition.

As used herein, the term “pharmaceutically active agent” means any substance or compound suitable for administration to a subject and furnishes biological activity or other direct effect in the treatment, cure, mitigation, diagnosis, or prevention of disease, or affects the structure or any function of the subject. Pharmaceutically active agents include, but are not limited to, substances and compounds described in the Physicians' Desk Reference (PDR Network, LLC; 64th edition; Nov. 15, 2009) and “Approved Drug Products with Therapeutic Equivalence Evaluations” (U.S. Department of Health and Human Services, 30^{th} edition, 2010), which are hereby incorporated by reference. Pharmaceutically active agents which have pendant carboxylic acid groups may be modified in accordance with the present disclosure using standard esterification reactions and methods readily available and known to those having ordinary skill in the art of chemical syntheses. Where a pharmaceutically active agent does not possess a carboxylic acid group, the ordinarily skilled artisan will be able to design and incorporate a carboxylic acid group into the pharmaceutically active agent where esterification may subsequently be carried out so long as the modification does not interfere with the pharmaceutically active agent's biological activity or effect.

The compounds used in the method of the present disclosure may be in a salt form. As used herein, a “salt” is a salt of the instant compounds which has been modified by making acid or base salts of the compounds. In the case of compounds used to treat a disease or medical disorder, the salt is pharmaceutically acceptable. Examples of pharmaceutically acceptable salts include, but are not limited to: mineral or organic acid salts of basic residues such as amines; alkali or organic salts of acidic residues such as phenols; and alkali or organic salts of acidic residues such as carboxylic acids. The salts can be made using an organic or inorganic acid. Such acid salts are chlorides, bromides, sulfates, nitrates, phosphates, sulfonates, formates, tartrates, maleates, malates, citrates, benzoates, salicylates, ascorbates, and the like. Phenolate salts are the sodium, potassium, or lithium salts, and the like. Carboxylate salts are the sodium, potassium, or lithium salts, and the like. The term “pharmaceutically acceptable salt” in this respect refers to the relatively non-toxic, inorganic or organic, acid or base addition salts of compounds of the present disclosure. These salts can be prepared in situ during the final isolation and purification of the compounds of the present disclosure, or by separately reacting a purified compound of the present disclosure in its free base or free acid form with a suitable organic or inorganic acid or base, and isolating the salt thus formed. Representative salts include hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts, and the like. (See, e.g., Berge et al. (1977) “Pharmaceutical Salts”, J. Pharm. Sci. 66:1-19).

A salt or pharmaceutically acceptable salt is contemplated for all compounds disclosed herein.

As used herein, “treating” means preventing, slowing, halting, or reversing the progression of a disease. Treating may also mean improving one or more symptoms of a disease.

The compounds used in the method of the present disclosure may be administered in various forms, including those detailed herein. The treatment with the compound may be a component of a combination therapy or an adjunct therapy, i.e., the subject or patient in need of the drug is treated with or given another drug for the disease in conjunction with one or more of the instant compounds. This combination therapy can be sequential therapy where the patient is treated first with one drug and then the other or the two drugs are given simultaneously. These can be administered independently by the same route or by two or more different routes of administration depending on the dosage forms employed.

As used herein, a “pharmaceutically acceptable carrier” is a pharmaceutically acceptable solvent, suspending agent, or vehicle, for delivering the instant compounds to the animal or human. The carrier may be liquid or solid and is selected with the planned manner of administration in mind. Liposomes are also a pharmaceutically acceptable carrier, as are capsules, coatings, and various syringes.

The dosage of the compounds administered in treatment will vary depending upon factors such as: the pharmacodynamic characteristics of a specific chemotherapeutic agent and its mode and route of administration; the age, sex, metabolic rate, absorptive efficiency, health and weight of the recipient; the nature and extent of the symptoms; the kind of concurrent treatment being administered; the frequency of treatment; and the desired therapeutic effect.

A dosage unit of the compounds used in the method of the present disclosure may comprise a single compound or mixtures thereof with additional agents. The compounds can be administered in oral dosage forms as tablets, capsules, pills, powders, granules, elixirs, tinctures, suspensions, syrups, and emulsions. The compounds may also be administered in intravenous (bolus or infusion), intraperitoneal, subcutaneous, or intramuscular form, or introduced directly, e.g., by injection, topical application, or other methods, into or onto a site of disease, all using dosage forms well known to those of ordinary skill in the pharmaceutical arts.

The compounds used in the method of the present disclosure can be administered in admixture with suitable pharmaceutical diluents, extenders, excipients, or carriers (collectively referred to herein as a pharmaceutically acceptable carrier) suitably selected with respect to the intended form of administration and as consistent with conventional pharmaceutical practices. The unit will be in a form suitable for oral, rectal, topical, intravenous, or direct injection or parenteral administration. The compounds can be administered alone or mixed with a pharmaceutically acceptable carrier. This carrier can be a solid or liquid, and the type of carrier is generally chosen based on the type of administration being used. The active agent can be co-administered in the form of a tablet or capsule, liposome, as an agglomerated powder, or in a liquid form. Examples of suitable solid carriers include lactose, sucrose, gelatin, and agar. Capsule or tablets can be easily formulated and can be made easy to swallow or chew. Other solid forms include granules and bulk powders. Tablets may contain suitable binders, lubricants, diluents, disintegrating agents, coloring agents, flavoring agents, flow-inducing agents, and melting agents. Examples of suitable liquid dosage forms include solutions or suspensions in water, pharmaceutically acceptable fats and oils, alcohols or other organic solvents, including esters, emulsions, syrups, or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents. Oral dosage forms optionally contain flavoring and coloring agents. Parenteral and intravenous forms may also include minerals and other materials to make them compatible with the type of injection or delivery system chosen.

Techniques and compositions for making dosage forms useful in the present disclosure are described in the following references: 7 Modern Pharmaceutics, Chapters 9 and 10 (Banker & Rhodes, Editors, 1979); Pharmaceutical Dosage Forms: Tablets (Lieberman et al. 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol. 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); and Modern Pharmaceutics Drugs and the Pharmaceutical Sciences, Vol 40 (Gilbert S. Banker, Christopher T. Rhodes, Eds.). All of the aforementioned publications are incorporated by reference herein.

Tablets may contain suitable binders, lubricants, disintegrating agents, coloring agents, flavoring agents, flow-inducing agents, and melting agents. For instance, for oral administration in the dosage unit form of a tablet or capsule, the active drug component can be combined with an oral, non-toxic, pharmaceutically acceptable, inert carrier such as lactose, gelatin, agar, starch, sucrose, glucose, methyl cellulose, magnesium stearate, dicalcium phosphate, calcium sulfate, mannitol, sorbitol, and the like. Suitable binders include starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

The compounds used in the method of the present disclosure may also be administered in the form of liposome delivery systems, such as small unilamellar vesicles, large unilamellar vesicles, and multilamellar vesicles. Liposomes can be formed from a variety of phospholipids, such as cholesterol, stearylamine, or phosphatidylcholines. The compounds may be administered as components of tissue-targeted emulsions.

The compounds used in the method of the present disclosure may also be coupled to soluble polymers as targetable drug carriers or as a prodrug. Such polymers include polyvinylpyrrolidone, pyran copolymer, polyhydroxylpropylmethacrylamide-phenol, polyhydroxyethylasparta-midephenol, or polyethyleneoxide-polylysine substituted with palmitoyl residues. Furthermore, the compounds may be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacylates, and crosslinked or amphipathic block copolymers of hydrogels.

Gelatin capsules may contain the active ingredient compounds and powdered carriers, such as lactose, starch, cellulose derivatives, magnesium stearate, stearic acid, and the like. Similar diluents can be used to make compressed tablets. Both tablets and capsules can be manufactured as immediate release products or as sustained release products to provide for continuous release of medication over a period of hours. Compressed tablets can be sugar coated or film coated to mask any unpleasant taste and protect the tablet from the atmosphere, or enteric coated for selective disintegration in the gastrointestinal tract.

For oral administration in liquid dosage form, the oral drug components are combined with any oral, non-toxic, pharmaceutically acceptable inert carrier such as ethanol, glycerol, water, and the like. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents.

Liquid dosage forms for oral administration can contain coloring and flavoring to increase patient acceptance. In general, water, a suitable oil, saline, aqueous dextrose (glucose), and related sugar solutions and glycols such as propylene glycol or polyethylene glycols are suitable carriers for parenteral solutions. Solutions for parenteral administration preferably contain a water-soluble salt of the active ingredient, suitable stabilizing agents, and if necessary, buffer substances. Antioxidizing agents such as sodium bisulfite, sodium sulfite, or ascorbic acid, either alone or combined, are suitable stabilizing agents. Also used are citric acid and its salts and sodium EDTA. In addition, parenteral solutions can contain preservatives, such as benzalkonium chloride, methyl- or propyl-paraben, and chlorobutanol. Suitable pharmaceutical carriers are described in Remington's Pharmaceutical Sciences, 17th ed., 1989, a standard reference text in this field.

The compounds used in the method of the present disclosure may also be administered in intranasal form via use of suitable intranasal vehicles, or via transdermal routes, using those forms of transdermal skin patches well known to those of ordinary skill in the art. To be administered in the form of a transdermal delivery system, the dosage administration will generally be continuous rather than intermittent throughout the dosage regimen.

Parenteral and intravenous forms may also include minerals and other materials to make them compatible with the type of injection or delivery system chosen.

Each embodiment disclosed herein is contemplated as being applicable to each of the other disclosed embodiments. Thus, all combinations of the various elements described herein are within the scope of the present disclosure. Any of the disclosed generic or specific compounds may be applicable to any of the disclosed compositions, processes, or methods.

The present disclosure will be better understood by reference to the Experimental Details that follow, but those skilled in the art will readily appreciate that the specific experiments detailed are only illustrative of the present disclosure as described more fully in the claims, which follow thereafter.

### Experimental Details

### Co-Administration of Bispecific RBP4/TTR Ligand ACPHS-52 and C20-D3-Retinyl Acetate Restored the Concentration of Functional Rhodopsin in Mice.

ACPHS-52 was previously identified and described as a compound that exemplifies a novel class of bispecific ligands that act as both RBP4 antagonists and kinetic stabilizers of TTR tetramers (Cioffi, C. L. et al. 2020). Thus, ACPHS-52 was used as a representative “first component” of the present disclosure in order to assess the feasibility of the co-drug strategy. Accordingly, an experiment was conducted to determine whether partial reduction in functional rhodopsin (opsin with conjugated 11-cis-retinal) induced by oral ACPHS-52 in mice can be compensated by co-administration with C20-D3-retinyl acetate. It was previously reported that ACPHS-52 can induce a pronounced reduction in serum RBP4 along with stabilization of TTR tetramers (Cioffi, C. L. et al. 2020).

The experiment was conducted in Balb/c mice. Three groups of mice were used: control untreated mice; mice treated with ACPHS-52; and mice treated with the ACPHS-52 plus C20-D3-retinyl acetate combination. ACPHS-52 was formulated into the chow to ensure a standard daily oral dosing of the compound at a 25 mg/kg dose. C20-D3-retinyl acetate was dissolved in olive oil and was administered through oral gavage daily at a 3.3 mg/kg dose. Dosing duration was 7 days. Following compound dosing, retinal extracts were prepared as previously described (Racz, B. et al. 2018). Rhodopsin was measured spectrophotometrically in retinal extracts (n=8) from dark-adapted treated and control mice using a previously published protocol (Racz, B. et al. 2018).

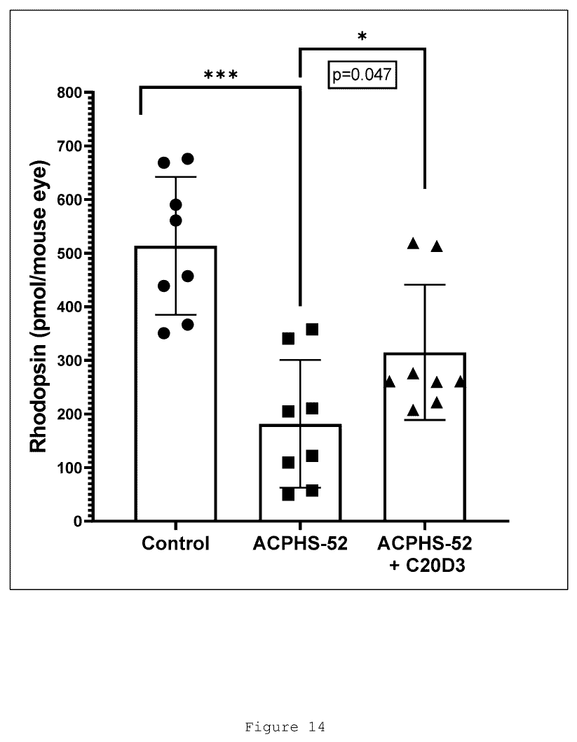

As shown in FIG. 14, ACPHS-52 given for 7 days to wild-type mice induced a 3-fold reduction in rhodopsin in comparison to untreated mice. However, an unpaired t-test on the ungrouped data revealed a statistically significant 1.8-fold increase in rhodopsin concentration in mice co-treated with C20-D3-retinyl acetate and ACPHS-52 in comparison to the ACPHS-52-treated group (P<0.05). Therefore, co-administration of ACPHS-52 with C20-D3-retinyl acetate restored the concentration of rhodopsin to ~65% of the normal level, proving that deuterated retinoids delivered to the retina by RBP4-independent routes can indeed compensate for the rhodopsin reduction induced by RBP4 lowering.

Partial reduction of rhodopsin associated with RBP4 lowering is at the core of mechanism-based ocular AEs (adverse effects) associated with pharmacological RBP4 reduction, and the experimental results show that co-administration with C20-D3-retinoid can partially restore the level of rhodopsin in ACPHS-52-treated mice.

Animal Care and Use Statement: All procedures are in compliance with: the U.S. Department of Agriculture's (USDA) Animal Welfare Act (9 CFR Parts 1, 2, and 3); the Guide for the Care and Use of Laboratory Animals, Institute of Laboratory Animal Resources, National Academy Press, Washington, D.C., 1996; and the National Institutes of Health, Office of Laboratory Animal Welfare. Whenever possible, procedures in this study are designed to avoid or minimize discomfort, distress, and pain to animals.

### DISCUSSION

Reduction of rhodopsin levels caused by pharmacological RBP4 reduction is at the core of mechanism-based ocular AEs such as nyctalopia, chromatopsia, and delayed dark adaptation. The present disclosure shows that co-administration of C20-D3-retinyl ester with the compound that induces a pronounced serum RBP4 reduction can partially restore the level of rhodopsin.

### REFERENCES

Adams, D. et al. Expert Opin. Pharmacother. 17, 791-802 (2016).
Adams, W. R. et al. Proc. Soc. Exp. Biol. Med. 208, 178-185 (1995).
Aeberli, I. et al. J. Clin. Endocrinol. Metab. 92, 4359-4365 (2007).
Alas, M. et al. J. Med. Chem. 64, 216-232 (2021).
Alexander, K. M. & Falk, R. H. Circ.: Heart Failure 9, e003489 (2016).
Almeida, M. R. et al. Biochem. J. 381, 351-356 (2004).
Almeida, M. R. et al. Curr. Drug Targets CNS Neurol. Disord. 4, 587-596 (2005).
Alnylam Announces 2022 Product and Pipeline Goals 2021, https://www.alnylam.com/sites/default/files/pdfs/Alnylam-Corporate-Presentation.pdf.
Ben-Shabat, S. et al. J. Biol. Chem. 277, 7183-7190 (2002).
Bergen, H. R. et al. J. Label. Compd. Radiopharm. 25, 11-21 (1988).
Bergmann, M. et al. FASEB J. 18, 562-564 (2004).
Berni, R. & Formelli, F. FEBS Lett. 308, 43-45 (1992).
Berk, J. L. et al. Amyloid 19, 37-38 (2012).
Birnbach, C. D. et al. Ophthalmology 101, 1211-1219 (1994).
Borhan, B. et al. Chem. Eur. 5, 1172-1175 (1999).
Boyer, N. P. et al. J. Biol. Chem. 287, 22276-22286 (2012).
Brandstetter, C. et al. J. Biol. Chem. 290, 31189-31198 (2015A).
Brandstetter, C. et al. J. Mol. Med. (Berl.) 93, 905-916 (2015B).
Brunjes, D. L. et al. J. Card. Failure 22, 996-1003 (2016).
Bulawa, C. E. et al. Proc. Natl. Acad. Sci. U.S.A. 109, 9629-9634 (2012).
Buxbaum, J. N. et al. Genet. Med. 19, 733-742 (2017).
Camerini, T. et al. J. Clin. Oncol. 19, 1664-1670 (2001).
Charbel, I. P. et al. Proc. Natl. Acad. Sci. U.S.A. 112, 8415-8420 (2015).
Cioffi, C. L. et al. J. Med. Chem. 57, 7731-7757 (2014).
Cioffi, C. L. et al. J. Med. Chem. 58, 5863-5888 (2015).
Cioffi, C. L. et al. J. Med. Chem. 62, 5470-5500 (2019).
Cioffi, C. L. et al. J. Med. Chem. 63, 11054-11084 (2020).
Cioffi, C. L. et al. J. Med. Chem. 64, 9010-9041 (2021).
Clagett-Dame, M. & DeLuca, H. F. Annu. Rev. Nutr. 22, 347-381 (2002).
Coelho, T. et al. Neurol. Ther. 5, 1-25 (2016).
Connelly, S. et al. Current opinion in structural biology 20, 54-62 (2010).
Connors, L. H. et al. Amyloid 18, 157-159 (2011).
De Laey, J. J. & Verougstraete, C. Retina (Philadelphia, Pa) 15, 399-406 (1995).
De, S. & Sakmar, T. P. J. Gen. Physiol. 120, 147-157 (2002).
Delori, F. C. in G. Coscas FCP, ed. Retinal Pigment Epithelium and Macular Disease (Documenta Ophthalmologica) Vol 62, 37-45 (1995) (Dordrecht, The Netherlands Kluwer Academic Publishers).
Dobri, N. et al. Invest. Ophth. Vis. Sci. 54, 85-95 (2013).
Dontsov, A. E. et al. Dokl. Biochem. Biophys. 425, 98-101 (2009).
Dorey, C. K. et al. Invest. Ophth. Vis. Sci. 30, 1691-1699 (1989).
Eagle, R. C. et al. Ophthalmology 87, 1189-1200 (1980).
Falk, R. H. et al. N. Engl. J. Med. 337, 898-909 (1997).
Finnemann, S. C. et al. Proc. Natl. Acad. Sci. U.S.A. 99, 3842-3847 (2002).
Finniss, M. C. et al. Med. Chem. Commun. 5, 1355-1358 (2014).
Foss, T. R. et al. Biochemistry 44, 15525-15533 (2005).
Geerlings, M. J. et al. Mol. Immunol. 84, 65-76 (2017).
Gliem, M. et al. JAMA Ophthalmol. 134, 817-824 (2016).
Goswami, B. C. & Barua, A. B. J. Label. Compd. Radiopharm. 46, 843-849 (2003).
Graham, T. E. et al. N. Engl. J. Med. 354, 2552-2563 (2006).
Haskell, M. J. et al. Am. J. Clin. Nutr. 66, 67-74 (1997).
Haskell, M. J. et al. Am. J. Clin. Nutr. 70, 874-880 (1999).
Hassan, W. et al. Texas Heart Institute journal/from the Texas Heart Institute of St. Luke's Episcopal Hospital, Texas Children's Hospital 32, 178-184 (2005).
Holz, F. G. et al. Graefes Arch. Clin. Exp. Ophthalmol. 237, 145-152 (1999).
Holz, F. G. et al. Invest. Ophth. Vis. Sci. 42, 1051-1056 (2001).
Holz, F. G. et al. Am. J. Ophthalmol. 143, 463-472 (2007).
Hyung, S. J. et al. ACS Chem. Biol. 5, 1137-1146 (2010).
Ingelsson, E. et al. Atherosclerosis 206, 239-244 (2009).
Johnson, S. M. et al. Acc. Chem. Res. 38, 911-921 (2005).
Judge, D. P. et al. J. Am. Coll. Cardiol. 74, 285-295 (2019).
Kaufman, Y. et al. J. Biol. Chem. 286, 7958-7965 (2011).
Keller, K. A. et al. Tetrahedron Lett. 46, 1181-1184 (2005).
Kennedy, C. J. et al. Eye (Lond.) 9, 763-771 (1995).
Kerschen, P. & Plante-Bordeneuve, V. Curr. Treat. Options Neurol. 18, 53 (2016).
Kiser, P. D. et al. Chem. Rev. 114, 194-232 (2014).
Klabunde, T. et al. Nat. Struct. Biol. 7, 312-321 (2000).
Kowalska, I. et al. J. Clin. Endocrinol. Metab. 93, 2786-2789 (2008).
Lamb, Y. N. & Deeks, E. D. Drugs 79, 863-874 (2019).
Lee, B. P. et al. Macromolecules 39, 1740-1748 (2006).
Lee, S. A. et al. Hepatology 64, 1534-1546 (2016).
Ma, L. et al. J. Biol. Chem. 286, 7966-7974 (2011).
Maeda, A. et al. Mol. Pharmacol. 70, 1220-1229 (2006).
Maeda, A. et al. Nat. Chem. Biol. 8, 170-178 (2012).
Mata, N. L. et al. Retina 33, 498-507 (2013).
Miller, M. et al. J. Med. Chem. 61, 7862-7876 (2018).
Miroy, G. J. et al. Proc. Natl. Acad. Sci. U.S.A. 93, 15051-15056 (1996).
Monteiro, C. et al. JCI Insight 4(12), e126526 (2019).
Motani, A. et al. J. Biol. Chem. 284, 7673-7680 (2009).
Nencetti, S. & Orlandini, E. Curr. Med. Chem. 19, 2356-2379 (2012).
Nencetti, S. et al. ChemMedChem 8, 1617-1619 (2013).
Nogrady, B. Nature 574, S8-S9 (2019).
Nollevaux, M. C. et al. Liver Int. 26, 182-186 (2006).
Norseen, J. et al. Mol. Cell. Biol. 32, 2010-2019 (2012).
O'Broin, C. Q. & Guiry, P. J. Org. Lett. 22, 879-883 (2020).
ONPATTRO® (patisiran) FULL PRESCRIBING INFORMATION 2018, https://www.accessdata.fda.gov/drugsatfda_docs/label/2018/210922s0001bl.pdf.
Palczewski, K. Trends Pharmacol. Sci. 31, 284-295 (2010).
Park, J. et al. Ann. Pharmacother. 54, 470-477 (2020).
Penchala, S. C. et al. Proc. Natl. Acad. Sci. U.S.A. 110, 9992-9997 (2013).
Petrassi, H. M. et al. J. Am. Chem. Soc. 127, 6662-6671 (2005).
Petrukhin, K., Expert Opin. Ther. Tar. 11, 625-639 (2007).
Petrukhin, K., Drug Discov. Today Ther. Strateg. 10, e11-e20 (2013).
Petrukhin, K. Recent Developments in Agents for the Treatment of Age-Related Macular Degeneration and Stargardt Disease. In: Cioffi, C. L. ed. Drug Delivery Challenges and Novel Therapeutic Approaches for Retinal Diseases. Cham: Springer International Publishing, 125-160 (2020).
Qi, Q. et al. J. Clin. Endocrinol. Metab. 92, 4827-4834 (2007).
Racz, B. et al. J. Biol. Chem. 293, 11574-11588 (2018).
Racz, B. et al. PLoS One 15, e0228291 (2020).
Radovic, B. et al. Br. J. Nutr. 95, 1171-1176 (2006).
Radu, R. A. et al. Proc. Natl. Acad. Sci. U.S.A. 100, 4742-4747 (2003).
Radu, R. A. et al. Invest. Ophth. Vis. Sci. 46, 4393-4401 (2005).
Radu, R. A. et al. J. Biol. Chem. 286, 18593-18601 (2011).
Radu, R. A. et al. J. Biol. Chem. 289, 9113-9120 (2014).
Raghu, P. et al. Arch. Biochem. Biophys. 400, 43-47 (2002).
Rodrigues, D. A. Drug-Drug Interactions, 2nd ed.; CRC Press: New York, 2008.
Rozanowska, M. et al. J. Biol. Chem. 270, 18825-18830 (1995).
Ruberg, F. L. & Berk, J. L. Circulation 126, 1286-1300 (2012).
Sant'Anna, R. et al. Nat. Commun. 7, 10787 (2016).
Schmitz-Valckenberg, S. et al. Surv. Ophthalmol. 54, 96-117 (2009).
Sekijima, Y. et al. Lab. Investig. 83, 409-417 (2003).
Smith, R. T. et al. Invest. Ophthalmol. Vis. Sci. 54, 5543 (2013).
Sparrow, J. R. et al. Invest. Ophth. Vis. Sci. 40, 2988-2995 (1999).
Sparrow, J. R. & Cai, B. Invest. Ophth. Vis. Sci. 42, 1356-1362 (2001)
Sparrow, J. R. et al. Invest. Ophth. Vis. Sci. 43, 1222-1227 (2002).
Sparrow, J. R. et al. Vis. Res. 43, 2983-2990 (2003).
Sparrow, J. R. & Boulton, M. Exp. Eye Res. 80, 595-606 (2005).
Sparrow, J. R. et al. Prog. Retin. Eye Res. 31, 121-135 (2012).
Sparrow, J. R. et al. Invest. Ophthalmol. Vis. Sci. 54, 8325-8326 (2013).
Steinmetz, A. C. et al. Annu. Rev. Biophys. Biomol. Struct. 30, 329-359 (2001).
Su, Z. et al. Acta Pharm. Sin. B. 11, 3889-3907 (2021).
Suter, M. et al. J. Biol. Chem. 275, 39625-39630 (2000).
Szebeni, J. Mol. Immunol. 61, 163-173 (2014).
Tang, P. H. et al. Prog. Retin. Eye Res. 2012.
Ton, V. K. et al. Clin. Med. Insights: Cardiol. 8s1, 39-44 (2014).
Tsin, A. et al. J. Biol. Chem. 293, 13016-13021 (2018).
Tsuchikama, K. & An, Z. Protein Cell 9, 33-46 (2018).
Ueda, M. et al. Modern pathology: an official journal of the United States and Canadian Academy of Pathology, Inc. 24, 1533-1544 (2011).
Vives-Bauza, C. et al. J. Biol. Chem. 283, 24770-24780 (2008).
Vogel, S. et al. Biochemistry 41, 15360-15368 (2002).
Washington, I. et al. Adv. Exp. Med. Biol. 854, 347-353 (2016).
Weng, J. et al. Cell 98, 13-23 (1999).
Westermark, P. et al. Amyloid: the international journal of experimental and clinical investigation: the official journal of the International Society of Amyloidosis 10 (Suppl 1), 48-54 (2003).
White, J. T. & Kelly, J. W. Proc. Natl. Acad. Sci. U.S.A. 98, 13019-13024 (2001).
Wolf, G. Physiological Rev. 64, 873-937 (1984).
Yang, Q. et al. Nature 436, 356-362 (2005).
Young, R. W. Surv. Ophthalmol. 31, 291-306 (1987).
Zhou, J. et al. Proc. Natl. Acad. Sci. U.S.A. 103, 16182-16187 (2006)

1. A compound having the general structure (P-)ₐM(-Q)_{b} or P(-Q)_{b}, wherein:
P represents a single-valence group formed by eliminating a hydrogen atom bonded to a heteroatom or by eliminating a hydroxyl group from a bispecific RBP4/TTR ligand;
a is 1, 2, 3, 4, or 5;
Q represents a single-valence group formed by eliminating a hydrogen atom bonded to a heteroatom or by eliminating a hydroxyl group from a C20-D3-visual-chromophore-producing compound;
b is 1, 2, 3, 4, or 5;
M represents an (a+b)-valence group comprising carbon, hydrogen, and oxygen atoms;
each P in (P-)ₐM(-Q)_{b} is independently bonded to M via an ester, carbamate, or hemiaminal linkage that is cleavable at an acidic pH or is enzymatically cleavable;
each Q in (P-)ₐM(-Q)_{b} is independently bonded to M via a carbonate, silyl ether, or ester linkage that is cleavable at an acidic pH or is enzymatically cleavable;
each Q in P(-Q)_{b} is independently bonded to P via an ester, carbamate, or hemiaminal linkage that is cleavable at an acidic pH or is enzymatically cleavable;
the bispecific RBP4/TTR ligand is a compound having the structure: 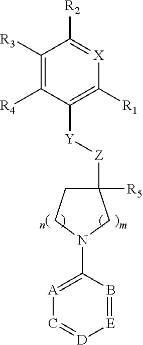
wherein
X is CR₆ or N;
R₁, R₂, R₃, R₄, and R₆ are each independently —H, —F, —Cl, —Br, —I, —NO₂, —CN, —CF₃, —CF₂H, —OCF₃, -(alkyl), -(haloalkyl), -(alkenyl), -(alkynyl), -(aryl), -(heteroaryl), -(cycloalkyl), -(cycloalkylalkyl), -(heteroalkyl), heterocycle, heterocycloalkyl, -(alkylheteroalkyl), -(alkylaryl), —OH, —OAc, —O-(alkyl), —O-(alkenyl), —O-(alkynyl), —O-(aryl), —O-(heteroaryl), —SH, —S-(alkyl), —S-(alkenyl), —S-(alkynyl), —S-(aryl), —S-(heteroaryl), —NH₂, —NH-(alkyl), —NH-(alkenyl), —NH-(alkynyl), —NH-(aryl), —NH-(heteroaryl), —C(O)R₇, —S(O)R₇, —SO₂R₇, —NHSO₂R₇, —OC(O)R₇, —SC(O)R₇, —NHC(O)R₈, or —NHC(S)R₈,
wherein R₇ is H, -(alkyl), —OH, —O(alkyl), —NH₂, —NH (alkyl), or —N(alkyl)₂, and
wherein R₈ is -(alkyl), —O-(alkyl), —NH₂, —NH(alkyl), or —N(alkyl)₂;
Y is O, S, N, NH, or a bond;
Z is O, S, N, NH, (CH₂)ₒ, or a bond;
R₅ is H, OH, halogen, or alkyl, or R₅ is (CH₂)ₚ and is bound to Y when Y is N to form a ring together with Z;
o and p are independently 0, 1, 2, or 3;
m and n are independently 0, 1, 2, 3, or 4;
A, C, and D are each independently N or CR₉;
R₉ is H, halogen, —OH, alkyl, cycloalkyl, cycloalkylalkyl, —O-(alkyl), —S-(alkyl), —NH₂, —NH(alkyl), —NH(alkyl)₂, —CO₂H, or —CO(O-alkyl);
B and E are N, CR₉, or C—F-G wherein at least one of B or E is C—F-G;
F is absent or present, and when present, is 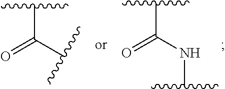
and
G is H, substituted or unsubstituted monocycle, bicycle, heteromonocycle, heterobicycle, aryl, heteroaryl, alkyl, cycloalkyl, cycloalkylalkyl, CO₂H, COOR₁₀, OH, OR₁₀, NH₂, NHR₁₀, NR₁₀R₁₁, SO₂ (alkyl), SO₂ (cycloalkyl), SO₂ (cycloalkylalkyl), CH₂NHR₁₀, CH₂NR₁₀R₁₁, or CH₂COOR₁₀,
wherein R₁₀ and R₁₁ are each independently H, alkyl, cycloalkyl, —C(O)-alkyl, —C(O)-cycloalkyl, —C(O)OH, —C(O)—O-alkyl, —C(O)—O— cycloalkyl, —C(O)NH₂, —C(O)NH(alkyl), —C(O)NH(cycloalkyl), —C(O) N(alkyl)₂, —CH₂NH (alkyl), —CH₂COOH, —SO₂CH₃, —OH, —O(alkyl), —NH₂, —NH(alkyl), or —N(alkyl)₂;
or a pharmaceutically acceptable salt thereof; and
the C20-D3-visual-chromophore-producing compound is selected from the group consisting of C20-D3-retinol, C20-D3-9-cis-retinol, C20-D3-11-cis-retinol, and C20-D3-C20′-D3-β-carotene.

2. The compound of claim 1, wherein the bispecific RBP4/TTR ligand is the compound wherein m is 1 or 2 and n is 0, 1, or 2;
preferably m is 1 and n is 1; and
wherein Y and Z are each independently CH₂, O, S, or NH,
preferably, Y is O and Z is CH₂,
or a pharmaceutically acceptable salt thereof.

3. (canceled)

4. The compound of claim 2, wherein the bispecific RBP4/TTR ligand is the compound wherein:
A and B are N, C and D are CR₉, and E is C—F-G; or
A, B, C, and D are CR₉, and E is C—F-G; or
A is N, B is C—F-G, and C, D, and E are each CR₉; or
A is N, B, C, and D are each CR₉, and E is C—F-G; or
A, C, and D are each CR₉, B is N, and E is C—F-G; and/or
X is CR₆ or N; and
R₁, R₂, R₃, R₄, and R₆ are each independently H, t-butyl, cyclopentyl, cyclohexyl, CF₃, F, Cl, CN, or —OCH₃; preferably,
wherein R₁ or R₄ is CF₃; more preferable,
X is CR₆;
R₁ is CF₃; and
R₂, R₃, R₄, and R₆ are each independently H, t-butyl, cyclopentyl, cyclohexyl, CF₃, F, Cl, CN, or —OCH₃,
or a pharmaceutically acceptable salt thereof.

5. The compound of claim 2, wherein the bispecific RBP4/TTR ligand has the structure: 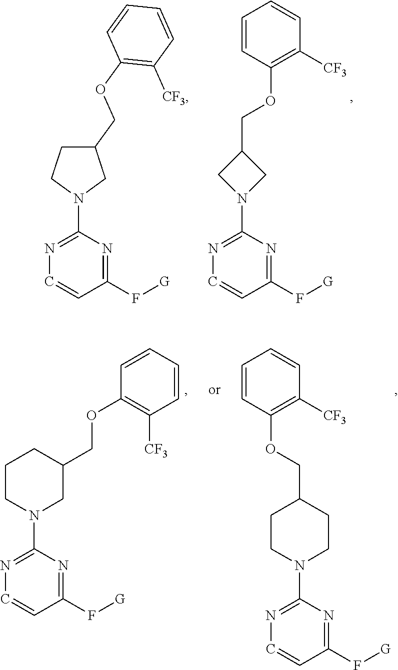
wherein
C is CR₉;
R₉ is H, halogen, —OH, alkyl, cycloalkyl, cycloalkylalkyl, —O-(alkyl), —S-(alkyl), —NH₂, —NH(alkyl), —NH(alkyl)₂, —CO₂H, or —CO(O-alkyl);
F is absent or present, and when present, is 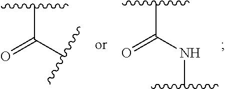
and
G is H, substituted or unsubstituted monocycle, bicycle, heteromonocycle, heterobicycle, aryl, heteroaryl, alkyl, cycloalkyl, cycloalkylalkyl, CO₂H, COOR₁₀, OH, OR₁₀, NH₂, NHR₁₀, NR₁₀R₁₁, SO₂ (alkyl), SO₂ (cycloalkyl), SO₂ (cycloalkylalkyl), CH₂NHR₁₀, CH₂NR₁₀R₁₁, or CH₂COOR₁₀,
wherein R₁₀ and R₁₁ are each independently H, alkyl, cycloalkyl, —C(O)-alkyl, —C(O)-cycloalkyl, —C(O)OH, —C(O)—O-alkyl, —C(O)—O-cycloalkyl, —C(O)NH₂, —C(O)NH(alkyl), —C(O)NH(cycloalkyl), —C(O) N(alkyl)₂, —CH₂NH (alkyl), —CH₂COOH, —SO₂CH₃, —OH, —O(alkyl), —NH₂, —NH(alkyl), or —N(alkyl)₂;
or a pharmaceutically acceptable salt thereof.

6. The compound of claim 5, wherein the bispecific RBP4/TTR ligand is the compound wherein:
C is CR₉; and
R₉ is H, -alkyl, —O(alkyl), or —NH(alkyl); preferably, R₉ is -alkyl,
or a pharmaceutically acceptable salt thereof.

7. The compound of claim 6, wherein the bispecific RBP4/TTR ligand has the structure 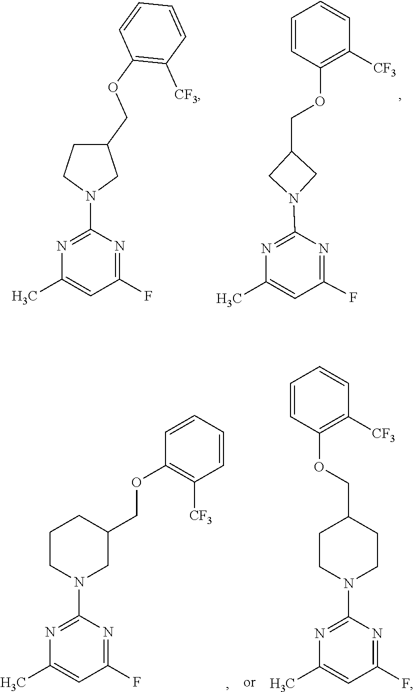
wherein F is a substituted or unsubstituted heteroaryl group, or
wherein F has the structure: 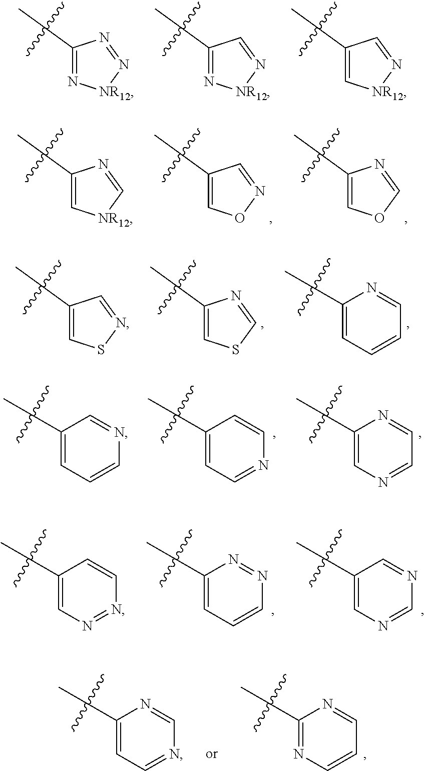
wherein R₁₂ is H, -(alkyl), -(alkenyl), or -(alkynyl), or a pharmaceutically acceptable salt thereof.

8. (canceled)

9. The compound of claim 1, wherein the bispecific RBP4/TTR ligand has the structure: 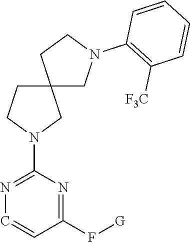
wherein
C is CR₉;
R₉ is H, halogen, —OH, alkyl, cycloalkyl, cycloalkylalkyl, —O-(alkyl), —S-(alkyl), —NH₂, —NH(alkyl), —NH(alkyl)₂, —CO₂H, or —CO(O-alkyl);
F is absent or present, and when present, is 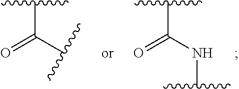
and
G is H, substituted or unsubstituted monocycle, bicycle, heteromonocycle, heterobicycle, aryl, heteroaryl, alkyl, cycloalkyl, cycloalkylalkyl, CO₂H, COOR₁₀, OH, OR₁₀, NH₂, NHR₁₀, NR₁₀R₁₁, SO₂ (alkyl), SO₂ (cycloalkyl), SO₂ (cycloalkylalkyl), CH₂NHR₁₀, CH₂NR₁₀R₁₁, or CH₂COOR₁₀,
wherein R₁₀ and R₁₁ are each independently H, alkyl, cycloalkyl, —C(O)-alkyl, —C(O)-cycloalkyl, —C(O)OH, —C(O)—O-alkyl, —C(O)—O— cycloalkyl, —C(O)NH₂, —C(O)NH(alkyl), —C(O)NH(cycloalkyl), —C(O)N(alkyl)₂, —CH₂NH(alkyl), —CH₂COOH, —SO₂CH₃, —OH, —O(alkyl), —NH₂, —NH(alkyl), or —N(alkyl)₂;
or a pharmaceutically acceptable salt thereof.

10. The compound of claim 9, wherein the bispecific RBP4/TTR ligand is the compound wherein C is CR₉ and R₉ is H, -alkyl, —O(alkyl), or —NH(alkyl),
or a pharmaceutically acceptable salt thereof.

11. The compound of claim 1, wherein the bispecific RBP4/TTR ligand has the structure 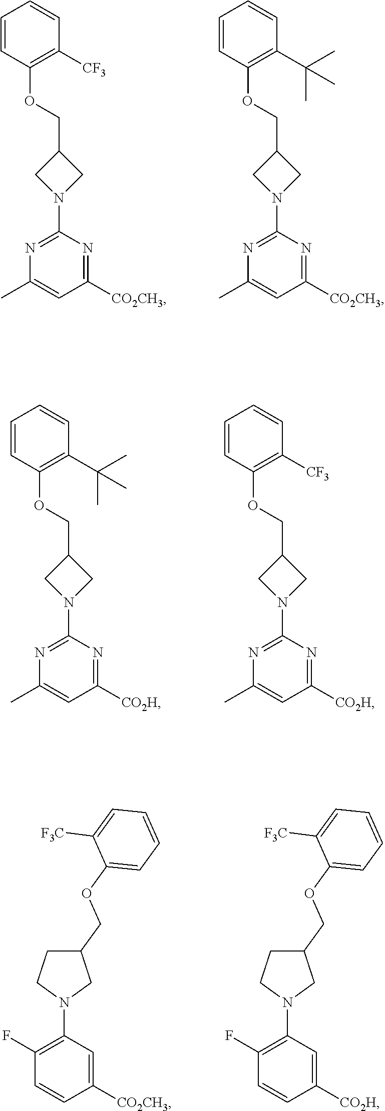 [SMILES: COC(=O)c1cc(C)nc(N2CC(COc3ccccc3C(F)(F)F)C2)n1 ; COC(=O)c1cc(C)nc(N2CC(COc3ccccc3C(C)(C)C)C2)n1 ; Cc1cc(C(=O)O)nc(N2CC(COc3ccccc3C(C)(C)C)C2)n1 ; Cc1cc(C(=O)O)nc(N2CC(COc3ccccc3C(F)(F)F)C2)n1 ; COC(=O)c1ccc(F)c(N2CCC(COc3ccccc3C(F)(F)F)C2)c1 ; O=C(O)c1ccc(F)c(N2CCC(COc3ccccc3C(F)(F)F)C2)c1] 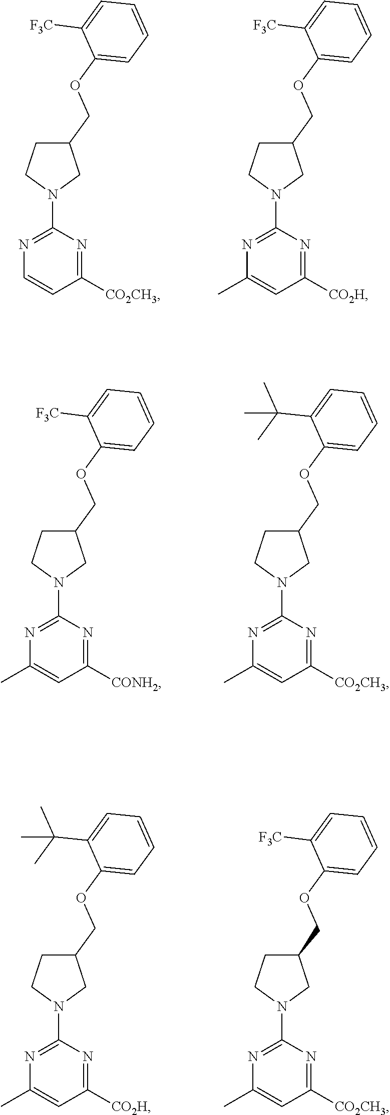 [SMILES: COC(=O)c1ccnc(N2CCC(COc3ccccc3C(F)(F)F)C2)n1 ; Cc1cc(C(=O)O)nc(N2CCC(COc3ccccc3C(F)(F)F)C2)n1 ; Cc1cc(C(N)=O)nc(N2CCC(COc3ccccc3C(F)(F)F)C2)n1 ; COC(=O)c1cc(C)nc(N2CCC(COc3ccccc3C(C)(C)C)C2)n1 ; Cc1cc(C(=O)O)nc(N2CCC(COc3ccccc3C(C)(C)C)C2)n1 ; COC(=O)c1cc(C)nc(N2CC[C@@H](COc3ccccc3C(F)(F)F)C2)n1] 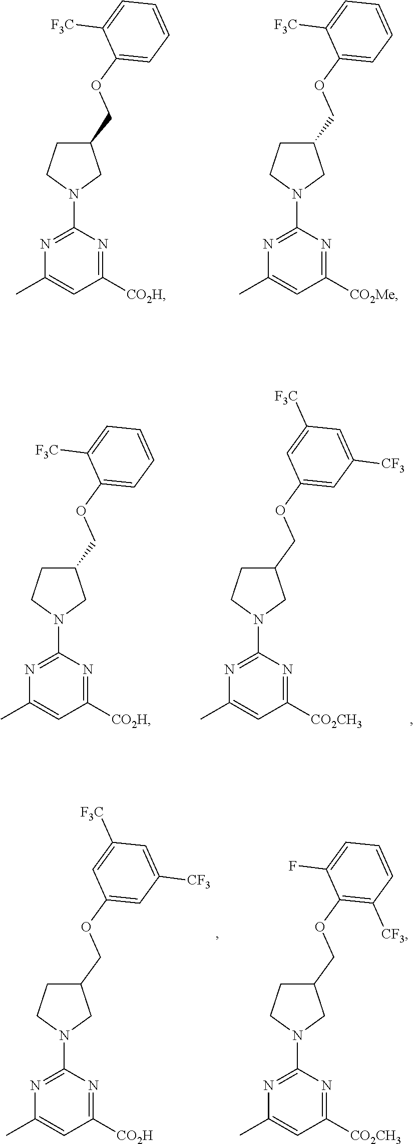 [SMILES: Cc1cc(C(=O)O)nc(N2CC[C@@H](COc3ccccc3C(F)(F)F)C2)n1 ; COC(=O)c1cc(C)nc(N2CC[C@H](COc3ccccc3C(F)(F)F)C2)n1 ; Cc1cc(C(=O)O)nc(N2CC[C@H](COc3ccccc3C(F)(F)F)C2)n1 ; COC(=O)c1cc(C)nc(N2CCC(COc3cc(C(F)(F)F)cc(C(F)(F)F)c3)C2)n1 ; Cc1cc(C(=O)O)nc(N2CCC(COc3cc(C(F)(F)F)cc(C(F)(F)F)c3)C2)n1 ; COC(=O)c1cc(C)nc(N2CCC(COc3c(F)cccc3C(F)(F)F)C2)n1] 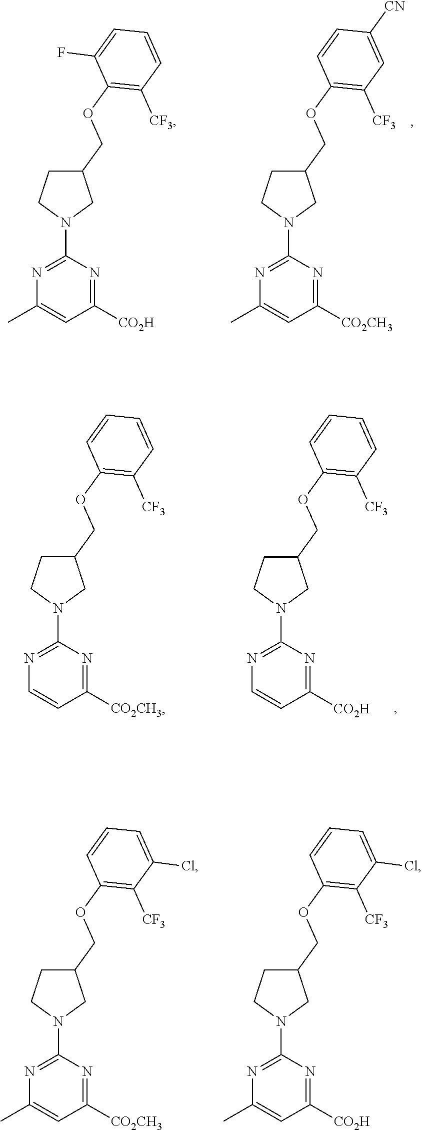 [SMILES: Cc1cc(C(=O)O)nc(N2CCC(COc3c(F)cccc3C(F)(F)F)C2)n1 ; COC(=O)c1cc(C)nc(N2CCC(COc3ccc(C#N)cc3C(F)(F)F)C2)n1 ; COC(=O)c1ccnc(N2CCC(COc3ccccc3C(F)(F)F)C2)n1 ; O=C(O)c1ccnc(N2CCC(COc3ccccc3C(F)(F)F)C2)n1 ; COC(=O)c1cc(C)nc(N2CCC(COc3cccc(Cl)c3C(F)(F)F)C2)n1 ; Cc1cc(C(=O)O)nc(N2CCC(COc3cccc(Cl)c3C(F)(F)F)C2)n1] 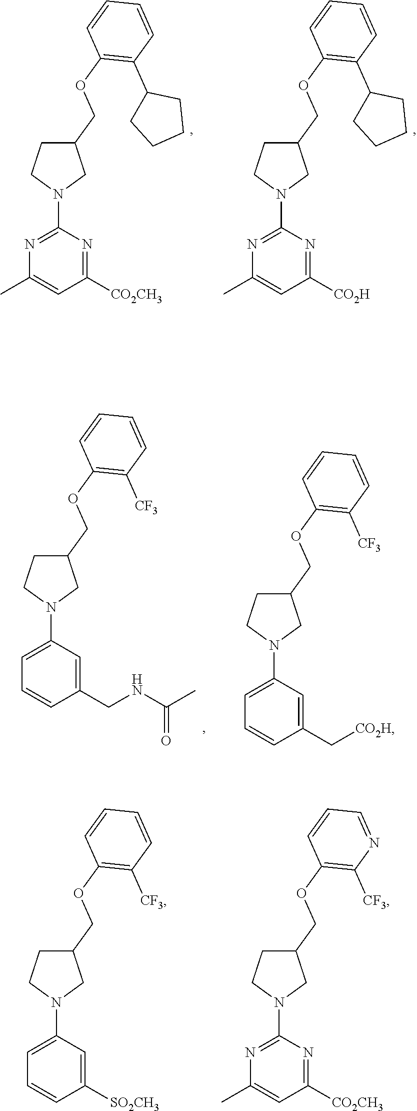 [SMILES: COC(=O)c1cc(C)nc(N2CCC(COc3ccccc3C3CCCC3)C2)n1 ; Cc1cc(C(=O)O)nc(N2CCC(COc3ccccc3C3CCCC3)C2)n1 ; CC(=O)NCc1cccc(N2CCC(COc3ccccc3C(F)(F)F)C2)c1 ; O=C(O)Cc1cccc(N2CCC(COc3ccccc3C(F)(F)F)C2)c1 ; CS(=O)(=O)c1cccc(N2CCC(COc3ccccc3C(F)(F)F)C2)c1 ; COC(=O)c1cc(C)nc(N2CCC(COc3cccnc3C(F)(F)F)C2)n1] 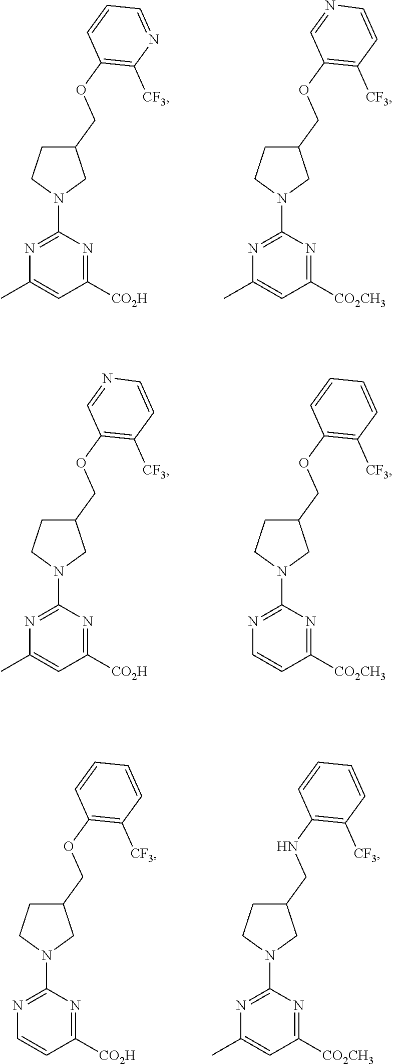 [SMILES: Cc1cc(C(=O)O)nc(N2CCC(COc3cccnc3C(F)(F)F)C2)n1 ; COC(=O)c1cc(C)nc(N2CCC(COc3cnccc3C(F)(F)F)C2)n1 ; Cc1cc(C(=O)O)nc(N2CCC(COc3cnccc3C(F)(F)F)C2)n1 ; COC(=O)c1ccnc(N2CCC(COc3ccccc3C(F)(F)F)C2)n1 ; O=C(O)c1ccnc(N2CCC(COc3ccccc3C(F)(F)F)C2)n1 ; COC(=O)c1cc(C)nc(N2CCC(CNc3ccccc3C(F)(F)F)C2)n1] 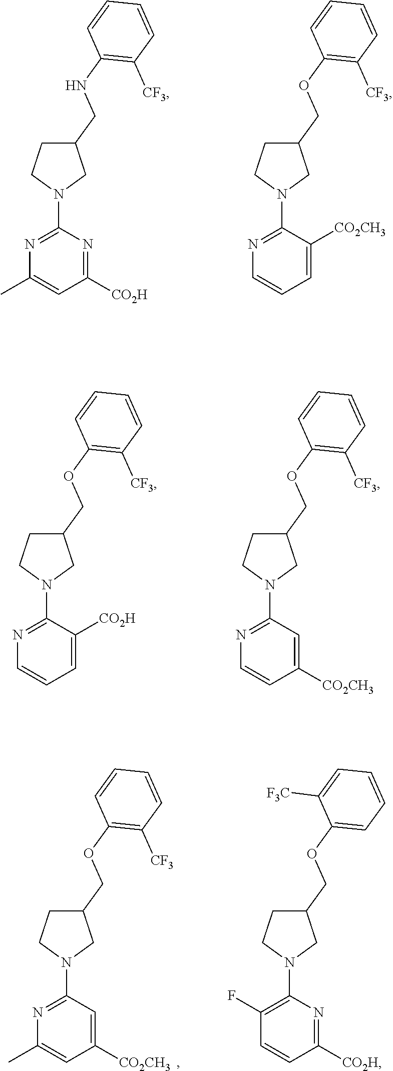 [SMILES: Cc1cc(C(=O)O)nc(N2CCC(CNc3ccccc3C(F)(F)F)C2)n1 ; COC(=O)c1cccnc1N1CCC(COc2ccccc2C(F)(F)F)C1 ; O=C(O)c1cccnc1N1CCC(COc2ccccc2C(F)(F)F)C1 ; COC(=O)c1ccnc(N2CCC(COc3ccccc3C(F)(F)F)C2)c1 ; COC(=O)c1cc(C)nc(N2CCC(COc3ccccc3C(F)(F)F)C2)c1 ; O=C(O)c1ccc(F)c(N2CCC(COc3ccccc3C(F)(F)F)C2)n1] 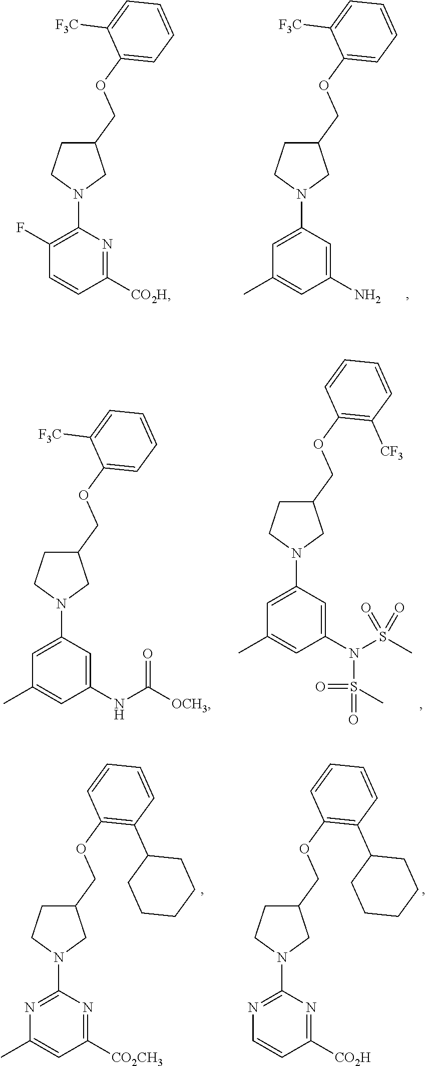 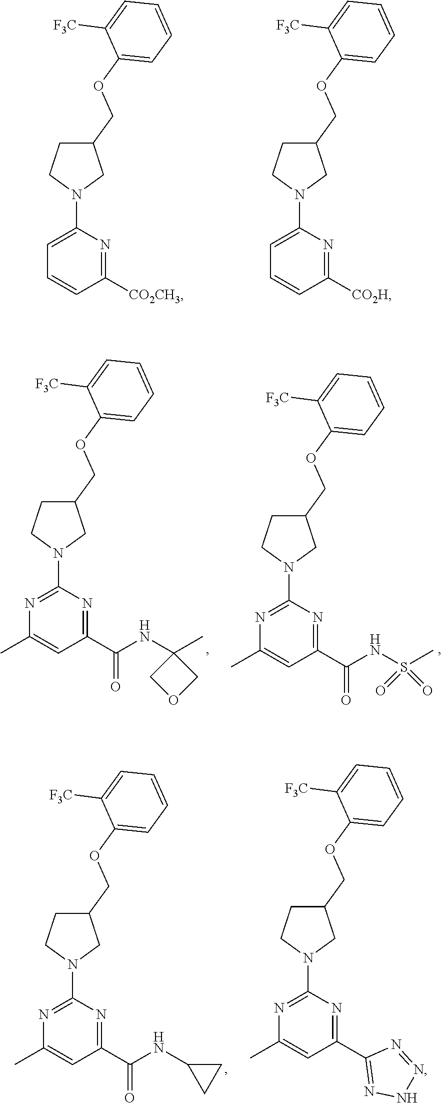 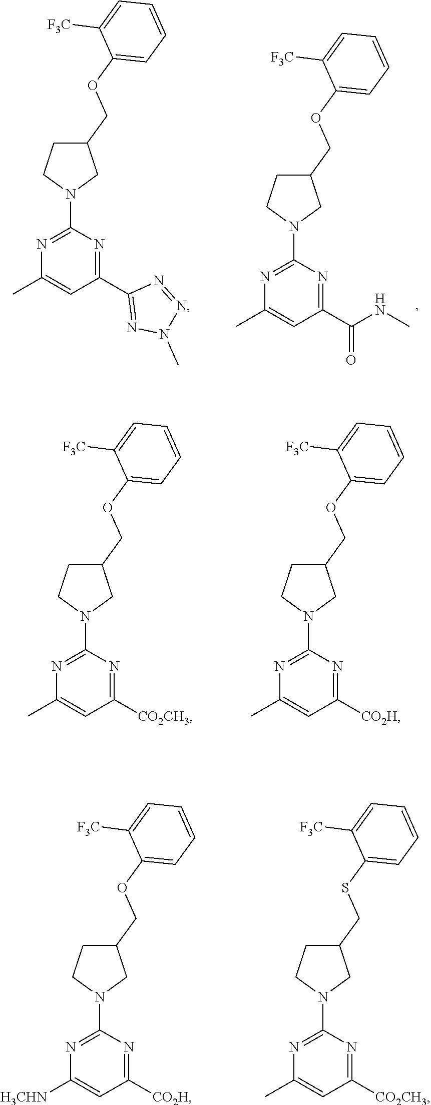 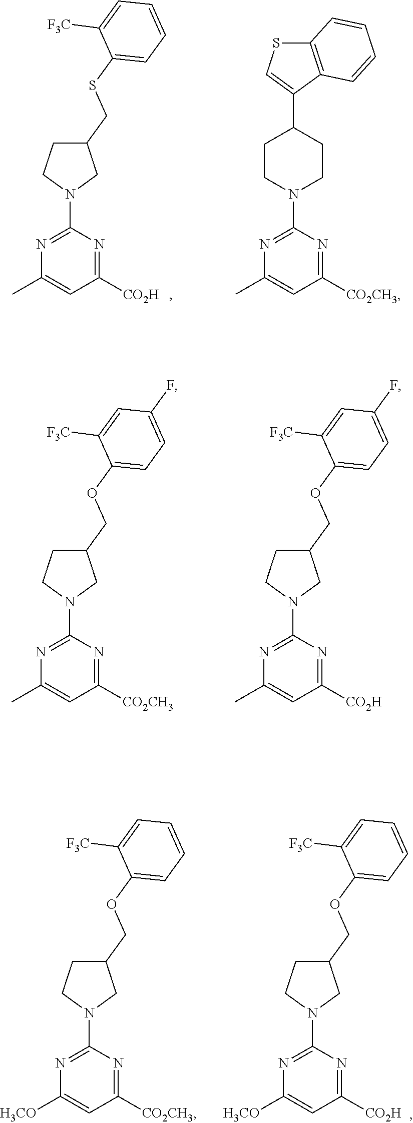 [SMILES: Cc1cc(C(=O)O)nc(N2CCC(CSc3ccccc3C(F)(F)F)C2)n1 ; COC(=O)c1cc(C)nc(N2CCC(c3csc4ccccc34)CC2)n1 ; COC(=O)c1cc(C)nc(N2CCC(COc3ccc(F)cc3C(F)(F)F)C2)n1 ; Cc1cc(C(=O)O)nc(N2CCC(COc3ccc(F)cc3C(F)(F)F)C2)n1 ; COC(=O)c1cc(OC)nc(N2CCC(COc3ccccc3C(F)(F)F)C2)n1 ; COc1cc(C(=O)O)nc(N2CCC(COc3ccccc3C(F)(F)F)C2)n1] 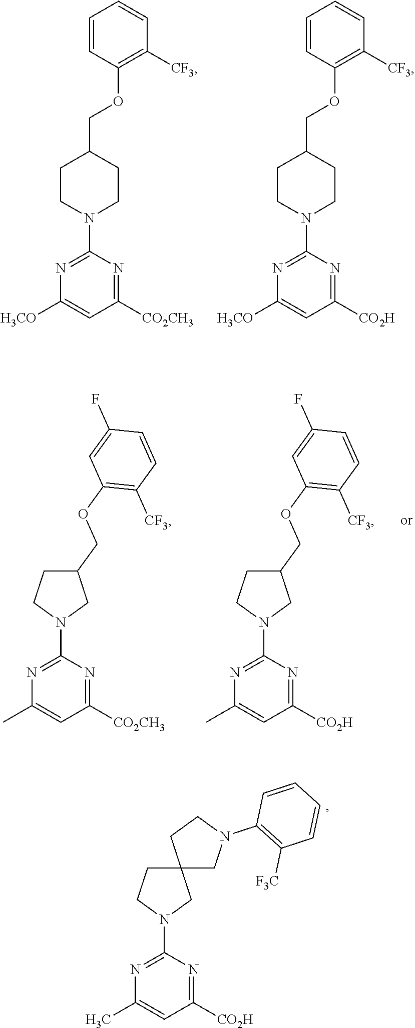 [SMILES: COC(=O)c1cc(OC)nc(N2CCC(COc3ccccc3C(F)(F)F)CC2)n1 ; COc1cc(C(=O)O)nc(N2CCC(COc3ccccc3C(F)(F)F)CC2)n1 ; COC(=O)c1cc(C)nc(N2CCC(COc3cc(F)ccc3C(F)(F)F)C2)n1 ; Cc1cc(C(=O)O)nc(N2CCC(COc3cc(F)ccc3C(F)(F)F)C2)n1 ; Cc1cc(C(=O)O)nc(N2CCC3(CCN(c4ccccc4C(F)(F)F)C3)C2)n1]
or a pharmaceutically acceptable salt of the compound.

12. The compound of claim 1, wherein the bispecific RBP4/TTR ligand has the structure 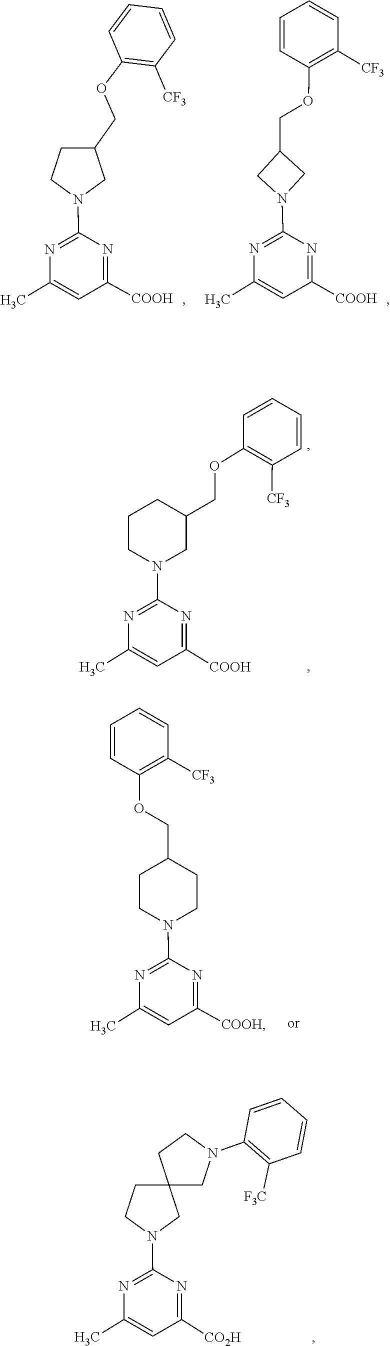
or a pharmaceutically acceptable salt of the compound.

13-16. (canceled)

17. The compound of claim 1, wherein:
a is 2 and b is 1; or
a is 1 and b is 1; or
a is 2, b is 1, and M is 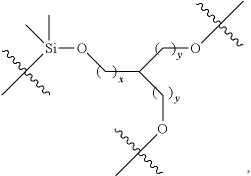
wherein x is 1, 2, 3, 4, or 5, and y is 1, 2, 3, 4, or 5; or
a is 2, b is 1, and M is 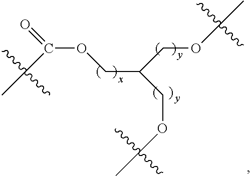
wherein x is 1, 2, 3, 4, or 5, and y is 1, 2, 3, 4, or 5; or
a is 2, b is 1, and M is 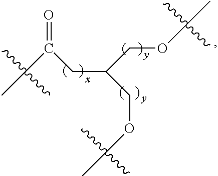
wherein x is 0, 1, 2, 3, or 4, and y is 1, 2, 3, 4, or 5;
or
a is 1, b is 1, and M is 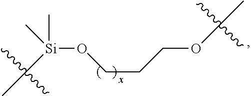
wherein x is 1, 2, 3, 4, or 5;
or
a is 1, b is 1, and M is 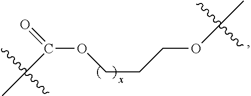
wherein x is 1, 2, 3, 4, or 5;
or
a is 1, b is 1, and M is 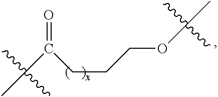
wherein x is 0, 1, 2, 3, or 4, and or
wherein P is 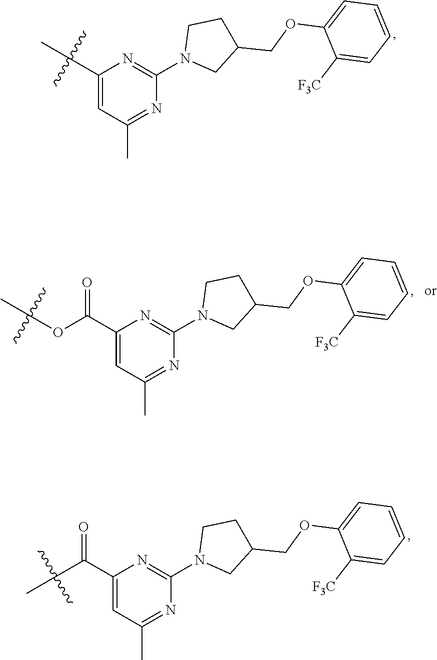 [SMILES: *c1cc(C)nc(N2CCC(COc3ccccc3C(F)(F)F)C2)n1 ; *OC(=O)c1cc(C)nc(N2CCC(COc3ccccc3C(F)(F)F)C2)n1 ; *C(=O)c1cc(C)nc(N2CCC(COc3ccccc3C(F)(F)F)C2)n1]
and/or
wherein Q is 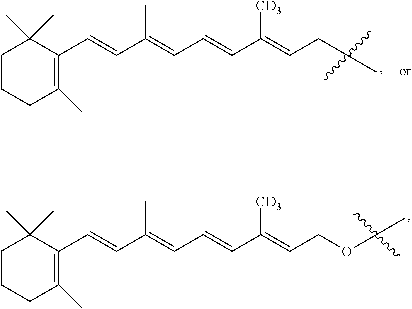 [SMILES: *C/C=C(/C=C/C=C(C)/C=C/C1=C(C)CCCC1(C)C)C([2H])([2H])[2H] ; *OC/C=C(/C=C/C=C(C)/C=C/C1=C(C)CCCC1(C)C)C([2H])([2H])[2H]]
and/or
wherein M is 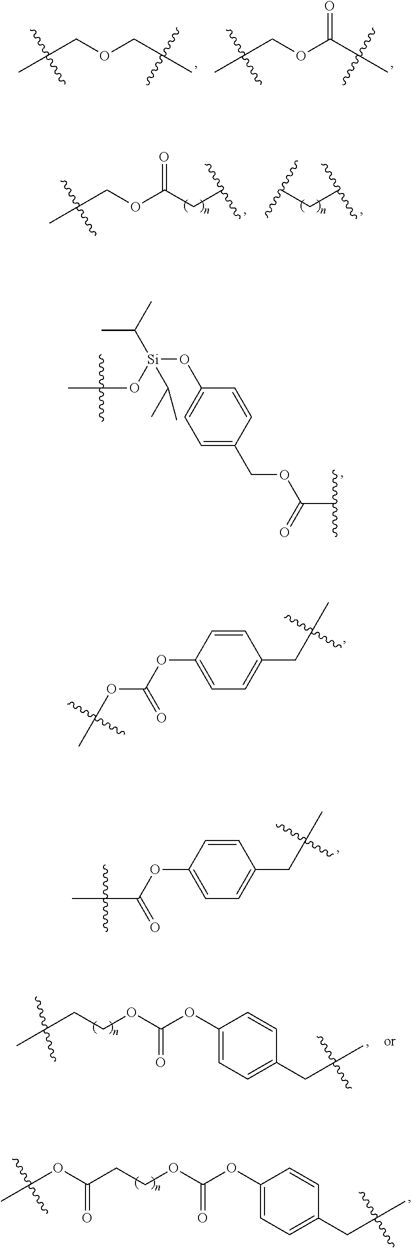
wherein n is 0-20,
or a pharmaceutically acceptable salt thereof.

18-20. (canceled)

21. The compound of claim 1 having the following structure: 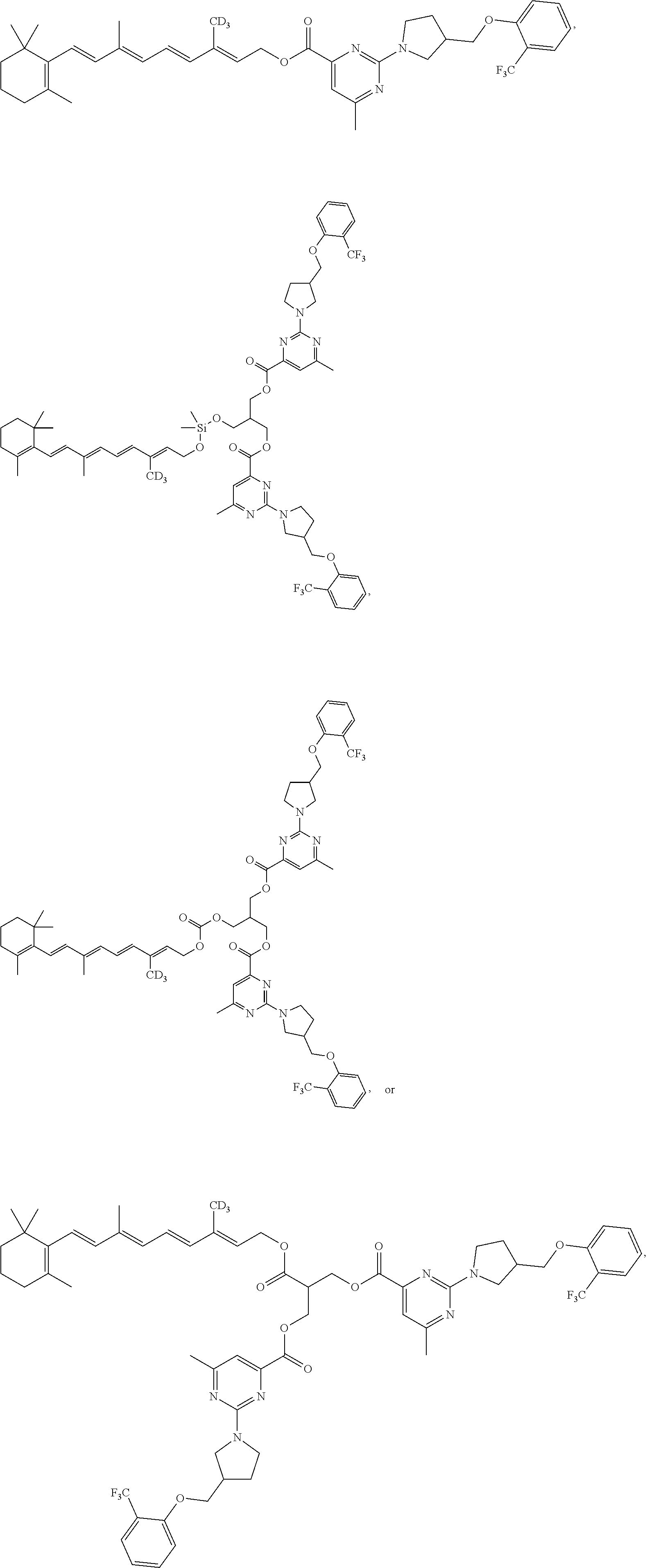
or a pharmaceutically acceptable salt thereof.

22. A pharmaceutical composition comprising a compound of claim 1 and a pharmaceutically acceptable carrier; or a pharmaceutical composition comprising the bispecific RBP4/TTR ligand from claims 1-21 and a C20-D3-visual-chromophore-producing compound, and a pharmaceutically acceptable carrier; preferably, the C20-D3-visual-chromophore-producing compound is selected from the group consisting of C20-D3-retinol, C20-D3-retinaldehyde, C20-D3-retinyl esters, C20-D3-9-cis-retinol, C20-D3-9-cis-retinaldehye, C20-D3-9-cis-retinyl esters, C20-D3-11-cis-retinol, C20-D3-11-cis-retinaldehye, C20-D3-11-cis-retinyl esters, and C20-D3-C20′-D3-β-carotene; more preferably, the C20-D3-visual-chromophore-producing compound is selected from the group consisting of C20-D3-retinol, C20-D3-9-cis-retinol, and C20-D3-11-cis-retinol.

23. A method for stabilizing TTR tetramers and for promoting rhodopsin and cone opsins production in a mammal, comprising administering to the mammal an amount of the pharmaceutical composition of claim 22 effective to stabilize TTR tetramers and to promote rhodopsin and cone opsins production; preferably, by a sequential, simultaneous, contemporaneous, or concomitant administration of the bispecific RBP4/TTR ligand and the C20-D3-visual-chromophore-producing compound; more preferably, in a sequential administration, the bispecific RBP4/TTR ligand may be administered first or the C20-D3-visual-chromophore-producing compound may be administered first.

24. A method for treating a disease characterized by excessive or age-related lipofuscin accumulation in the retina, or a TTR amyloidosis (ATTR) disease, or both a disease characterized by excessive or age-related lipofuscin accumulation and a TTR amyloidosis (ATTR) disease, in a mammal afflicted therewith, and for promoting rhodopsin and cone opsins production, comprising administering to the mammal an effective amount of the pharmaceutical composition of claim 22; preferably, by a sequential, simultaneous, contemporaneous, or concomitant administration of the bispecific RBP4/TTR ligand and the C20-D3-visual-chromophore-producing compound; more preferably, in a sequential administration, the bispecific RBP4/TTR ligand may be administered first or the C20-D3-visual-chromophore-producing compound may be administered first,
preferably, wherein the disease is further characterized by bisretinoid-mediated macular degeneration, or
wherein the disease characterized by age-related lipofuscin accumulation in the retina is
(a) Age-Related Macular Degeneration;
(b) dry (atrophic) Age-Related Macular Degeneration;
(c) Stargardt Disease or other forms of retinopathy caused by or associated with mutations in the ABCA4 gene, such as retinitis pigmentosa (RP19) or cone-rod dystrophy (CORD3);
(d) Best disease;
(e) adult vitelliform maculopathy; or
(f) Stargardt-like macular dystrophy.

25. (canceled)

26. The method of claim 23, wherein the amount of the compound or the amount of the pharmaceutical composition is effective to lower the serum concentration of RBP4 in the mammal, or wherein the amount of the compound or the amount of the pharmaceutical composition is effective to lower the retinal concentration of a bisretinoid in lipofuscin in the mammal; or wherein the amount of the compound or the amount of the pharmaceutical composition is effective to stabilize TTR tetramers in the mammal.
preferably, wherein the bisretinoid is A2E, isoA2E, A2-DHP-PE, or atRAL di-PE.

27-29. (canceled)

30. The method of claim 23, wherein the administration is effective to reduce photoreceptor degeneration; or
wherein the method is further effective to stabilize TTR tetramers in the mammal; or
wherein the mammal is further afflicted with a TTR amyloidosis (ATTR) disease and the method is effective for treating the TTR amyloidosis (ATTR) disease in the mammal.

31-32. (canceled)

33. The method of claim 30, wherein the TTR amyloidosis (ATTR) disease is senile systemic amyloidosis (SSA); peripheral polyneuropathy (ATTR-PN); cardiomyopathy (ATTR-CM); or the TTR amyloidosis (ATTR) disease is characterized by deposition of amyloid aggregates.

34. A method for
(a) treating type 2 diabetes and for promoting rhodopsin and cone opsins production in a mammal, comprising administering to the mammal an amount of the compound of any one of claims 1-21 or an amount of the pharmaceutical composition of claim 22 effective to treat type 2 diabetes and to promote rhodopsin and cone opsins production; preferably, by a sequential, simultaneous, contemporaneous, or concomitant administration of the bispecific RBP4/TTR ligand and the C20-D3-visual-chromophore-producing compound; more preferably, in a sequential administration, the bispecific RBP4/TTR ligand may be administered first or the C20-D3-visual-chromophore-producing compound may be administered first,
(b) treating obesity and for promoting rhodopsin and cone opsins production in a mammal, comprising administering to the mammal an amount of the compound of any one of claims 1-21 or an amount of the pharmaceutical composition of claim 22 effective to treat obesity and to promote rhodopsin and cone opsins production; preferably, by a sequential, simultaneous, contemporaneous, or concomitant administration of the bispecific RBP4/TTR ligand and the C20-D3-visual-chromophore-producing compound; more preferably, in a sequential administration, the bispecific RBP4/TTR ligand may be administered first or the C20-D3-visual-chromophore-producing compound may be administered first,
(c) treating insulin resistance and for promoting rhodopsin and cone opsins production in a mammal, comprising administering to the mammal an amount of the compound of any one of claims 1-21 or an amount of the pharmaceutical composition of claim 22 effective to treat insulin resistance and to promote rhodopsin and cone opsins production; preferably, by a sequential, simultaneous, contemporaneous, or concomitant administration of the bispecific RBP4/TTR ligand and the C20-D3-visual-chromophore-producing compound; more preferably, in a sequential administration, the bispecific RBP4/TTR ligand may be administered first or the C20-D3-visual-chromophore-producing compound may be administered first,
(d) treating cardiovascular disease and for promoting rhodopsin and cone opsins production in a mammal, comprising administering to the mammal an amount of the compound of any one of claims 1-21 or an amount of the pharmaceutical composition of claim 22 effective to treat cardiovascular disease and to promote rhodopsin and cone opsins production; preferably, by a sequential, simultaneous, contemporaneous, or concomitant administration of the bispecific RBP4/TTR ligand and the C20-D3-visual-chromophore-producing compound; more preferably, in a sequential administration, the bispecific RBP4/TTR ligand may be administered first or the C20-D3-visual-chromophore-producing compound may be administered first,
(e) treating hepatic steatosis and for promoting rhodopsin and cone opsins production in a mammal, comprising administering to the mammal an amount of the compound of any one of claims 1-21 or an amount of the pharmaceutical composition of claim 22 effective to treat hepatic steatosis and to promote rhodopsin and cone opsins production; preferably, by a sequential, simultaneous, contemporaneous, or concomitant administration of the bispecific RBP4/TTR ligand and the C20-D3-visual-chromophore-producing compound; more preferably, in a sequential administration, the bispecific RBP4/TTR ligand may be administered first or the C20-D3-visual-chromophore-producing compound may be administered first,
(f) A method for treating non-alcoholic fatty liver disease (NAFLD) and for promoting rhodopsin and cone opsins production in a mammal, comprising administering to the mammal an amount of the compound of any one of claims 1-21 or an amount of the pharmaceutical composition of claim 22 effective to treat non-alcoholic fatty liver disease (NAFLD) and to promote rhodopsin and cone opsins production; preferably, by a sequential, simultaneous, contemporaneous, or concomitant administration of the bispecific RBP4/TTR ligand and the C20-D3-visual-chromophore-producing compound; more preferably, in a sequential administration, the bispecific RBP4/TTR ligand may be administered first or the C20-D3-visual-chromophore-producing compound may be administered first.

35-39. (canceled)

40. A method for producing a compound having the structure (P-)₂M-Q wherein:
P represents a single-valence group formed by eliminating a hydroxyl group from a bispecific RBP4/TTR ligand;
Q represents a single-valence group formed by eliminating a hydrogen atom bonded to a heteroatom from a C20-D3-visual-chromophore-producing compound;
M represents a triple-valence group that is 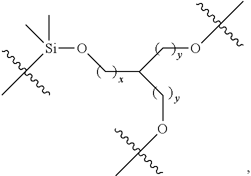
wherein x is 1 and y is 1;
each P is bonded to M via an ester linkage that is cleavable at an acidic pH or is enzymatically cleavable;
Q is bonded to M via a silyl ether linkage that is cleavable at an acidic pH or is enzymatically cleavable;
the bispecific RBP4/TTR ligand is a compound having the structure: 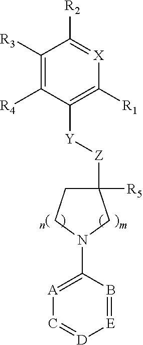
wherein
X is CR₆ or N;
R₁, R₂, R₃, R₄, and R₆ are each independently —H, —F, —Cl, —Br, —I, —NO₂, —CN, —CF₃, —CF₂H, —OCF₃, -(alkyl), -(haloalkyl), -(alkenyl), -(alkynyl), -(aryl), -(heteroaryl), -(cycloalkyl), -(cycloalkylalkyl), -(heteroalkyl), heterocycle, heterocycloalkyl, -(alkylheteroalkyl), -(alkylaryl), —OH, —OAc, —O-(alkyl), —O-(alkenyl), —O-(alkynyl), —O-(aryl), —O-(heteroaryl), —SH, —S-(alkyl), —S-(alkenyl), —S-(alkynyl), —S-(aryl), —S-(heteroaryl), —NH₂, —NH-(alkyl), —NH-(alkenyl), —NH-(alkynyl), —NH-(aryl), —NH-(heteroaryl), —C(O)R₇, —S(O)R₇, —SO₂R₇, —NHSO₂R₇, —OC(O)R₇, —SC(O)R₇, —NHC(O)R₈, or —NHC(S)R₈,
wherein R₇ is H, -(alkyl), —OH, —O(alkyl), —NH₂, —NH(alkyl), or —N(alkyl)₂, and
wherein R₈ is -(alkyl), —O-(alkyl), —NH₂, —NH(alkyl), or —N(alkyl)₂;
Y is O, S, N, NH, or a bond;
Z is O, S, N, NH, (CH₂)ₒ, or a bond;
R₅ is H, OH, halogen, or alkyl, or R₅ is (CH₂)ₚ and is bound to Y when Y is N to form a ring together with Z;
o and p are independently 0, 1, 2, or 3;
m and n are independently 0, 1, 2, 3, or 4;
A, C, and D are each independently N or CR₉;
R₉ is H, halogen, —OH, alkyl, cycloalkyl, cycloalkylalkyl, —O-(alkyl), —S-(alkyl), —NH₂, —NH(alkyl), —NH(alkyl)₂, —CO₂H, or —CO(O-alkyl);
B is N, CR₉, or C—F-G;
F is absent or present, and when present, is 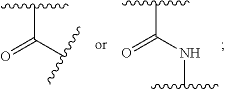
G is H, substituted or unsubstituted monocycle, bicycle, heteromonocycle, heterobicycle, aryl, heteroaryl, alkyl, cycloalkyl, cycloalkylalkyl, CO₂H, COOR₁₀, OH, OR₁₀, NH₂, NHR₁₀, NR₁₀R₁₁, SO₂ (alkyl), SO₂ (cycloalkyl), SO₂ (cycloalkylalkyl), CH₂NHR₁₀, CH₂NR₁₀R₁₁, or CH₂COOR₁₀,
wherein R₁₀ and R₁₁ are each independently H, alkyl, cycloalkyl, —C(O)-alkyl, —C(O) -cycloalkyl, —C(O)OH, —C(O)—O-alkyl, —C(O)—O-cycloalkyl, —C(O)NH₂, —C(O)NH(alkyl), —C(O)NH(cycloalkyl), —C(O)N(alkyl)₂, —CH₂NH(alkyl), —CH₂COOH, —SO₂CH₃, —OH, —O(alkyl), —NH₂, —NH(alkyl), or —N(alkyl)₂; and
E is C—F—CO₂H;
or a pharmaceutically acceptable salt thereof; and
the C20-D3-visual-chromophore-producing compound is selected from the group consisting of C20-D3-retinol, C20-D3-9-cis-retinol, and C20-D3-11-cis-retinol;
the method comprising the steps of:
modifying one of three hydroxyl groups of 2-(hydroxymethyl)-1,3-propanediol by a protecting group;
performing a dehydration reaction between the protecting-group-modified 2-(hydroxymethyl)-1,3-propanediol and the bispecific RBP4/TTR ligand to form a diester product;
removing the protecting group from the diester product; and
reacting the deprotected diester product with dichlorodimethylsilane and the C20-D3-visual-chromophore-producing compound to form the compound (P-)₂M-Q, and/or
wherein:
P represents a single-valence group formed by eliminating a hydroxyl group from a bispecific RBP4/TTR ligand;
Q represents a single-valence group formed by eliminating a hydrogen atom bonded to a heteroatom from a C20-D3-visual-chromophore-producing compound;
M represents a triple-valence group that is 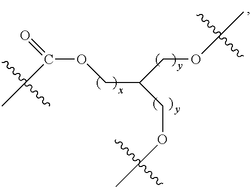
wherein x is 1 and y is 1;
each P is bonded to M via an ester linkage that is cleavable at an acidic pH or is enzymatically cleavable;
Q is bonded to M via a carbonate linkage that is cleavable at an acidic pH or is enzymatically cleavable;
the bispecific RBP4/TTR ligand is a compound having the structure: 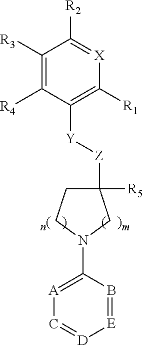
wherein
X is CR₆ or N;
R₁, R₂, R₃, R₄, and R₆ are each independently —H, —F, —Cl, —Br, —I, —NO₂, —CN, —CF₃, —CF₂H, —OCF₃, -(alkyl), -(haloalkyl), -(alkenyl), -(alkynyl), -(aryl), -(heteroaryl), -(cycloalkyl), -(cycloalkylalkyl), -(heteroalkyl), heterocycle, heterocycloalkyl, -(alkylheteroalkyl), -(alkylaryl), —OH, —OAc, —O-(alkyl), —O-(alkenyl), —O-(alkynyl), —O-(aryl), —O-(heteroaryl), —SH, —S-(alkyl), —S-(alkenyl), —S-(alkynyl), —S-(aryl), —S-(heteroaryl), —NH₂, —NH-(alkyl), —NH-(alkenyl), —NH-(alkynyl), —NH-(aryl), —NH-(heteroaryl), —C(O)R₇, —S(O)R₇, —SO₂R₇, —NHSO₂R₇, —OC(O)R₇, —SC(O)R₇, —NHC(O)R₈, or —NHC(S)R₈, wherein R₇ is H, -(alkyl), —OH, —O(alkyl), —NH₂, —NH(alkyl), or —N(alkyl)₂, and
wherein R₈ is -(alkyl), —O-(alkyl), —NH₂, —NH(alkyl), or —N(alkyl)₂;
Y is O, S, N, NH, or a bond;
Z is O, S, N, NH, (CH₂)ₒ, or a bond;
R₅ is H, OH, halogen, or alkyl, or R₅ is (CH₂)ₚ and is bound to Y when Y is N to form a ring together with Z;
o and p are independently 0, 1, 2, or 3;
m and n are independently 0, 1, 2, 3, or 4;
A, C, and D are each independently N or CR₉;
R₉ is H, halogen, —OH, alkyl, cycloalkyl, cycloalkylalkyl, —O-(alkyl), —S-(alkyl), —NH₂, —NH(alkyl), —NH(alkyl)₂, —CO₂H, or —CO(O-alkyl);
B is N, CR₉, or C—F-G;
F is absent or present, and when present, is 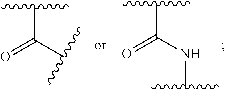
G is H, substituted or unsubstituted monocycle, bicycle, heteromonocycle, heterobicycle, aryl, heteroaryl, alkyl, cycloalkyl, cycloalkylalkyl, CO₂H, COOR₁₀, OH, OR₁₀, NH₂, NHR₁₀, NR₁₀R₁₁, SO₂ (alkyl), SO₂ (cycloalkyl), SO₂ (cycloalkylalkyl), CH₂NHR₁₀, CH₂NR₁₀R₁₁, or CH₂COOR₁₀,
wherein R₁₀ and R₁₁ are each independently H, alkyl, cycloalkyl, —C(O)-alkyl, —C(O) -cycloalkyl, —C(O)OH, —C(O)—O-alkyl, —C(O)—O-cycloalkyl, —C(O)NH₂, —C(O)NH(alkyl), —C(O)NH(cycloalkyl), —C(O)N(alkyl)₂, —CH₂NH (alkyl), —CH₂COOH, —SO₂CH₃, —OH, —O(alkyl), —NH₂, —NH(alkyl), or —N(alkyl)₂; and
E is C—F—CO₂H;
or a pharmaceutically acceptable salt thereof; and
the C20-D3-visual-chromophore-producing compound is selected from the group consisting of C20-D3-retinol, C20-D3-9-cis-retinol, and C20-D3-11-cis-retinol;
the method comprising the steps of:
performing a dehydration reaction between 2-(benzyloxymethyl)-1,3-propanediol and the bispecific RBP4/TTR ligand or a derivative thereof modified by protecting groups, to form a diester product;
reacting the diester product with the C20-D3-visual-chromophore-producing compound to form a mono-carbonate-linked diester product; and
removing the protecting groups, if present, from the mono-carbonate-linked diester product;
to form the compound (P-)₂M-Q.

41. (canceled)
